(19) United States
(12) Patent Application Publication
(10) Pub. No.: US 2010/0028326 A1
(43) Pub. Date: Feb. 4, 2010

(54) DIAGNOSIS OF HYPERINSULINEMIA AND TYPE II DIABETES AND PROTECTION AGAINST SAME BASED ON PROTEINS DIFFERENTIALLY EXPRESSED IN SERUM

(75) Inventors: John J. Kopchick, Athens, OH (US); Shigeru Okada, Athens, OH (US); Sudha Sankaran, Athens, OH (US)

Correspondence Address: WOOD, HERRON & EVANS, LLP, 2700 CAREW TOWER, 441 VINE STREET, CINCINNATI, OH, 45202, US

(73) Assignee: Ohio University, Athens, GA (US)

(21) Appl. No.: 11/721,106

(22) PCT Filed: Dec. 7, 2005
(86) PCT No.: PCT/US05/44182

§ 371 Date: Sep. 30, 2009

Related U.S. Application Data
(60) Provisional application No. 60/633,457, filed on Dec. 7, 2004.

(51) Int. Cl. A61K 39/395; A61K 38/16; A61K 31/7088; A61P 3/10

(52) U.S. Cl. 424/130.1; 514/12; 514/44 R

(57) ABSTRACT

Mouse proteins differentially expressed in serum, in comparisons of normal vs. hyperinsulinemic, hyperinsulinemic vs. type 2 diabetic, and normal vs. type 2 diabetic white adipose tissue have been identified, as have corresponding human proteins. The human molecules, or antagonists thereof, may be used for protection against hyperinsulinemia or type 2 diabetes, or their sequalae.

This application claims benefit under 35 USC 119(e) of prior U.S. provisional application 60/633,457, filed Dec. 7, 2004, hereby incorporated by reference in its entirety.

### CROSS-REFERENCE TO RELATED APPLICATIONS

Ohio University has filed a series of applications relating to genomic studies of differential expression of mouse genes in various tissues as a result of hyperinsulinemia or diabetes. None of these relate to differential expression in serum. It has also filed a series of applications relating to genomic studies of the effect of aging on the differential expression of mouse genes. Any reference in this application to “genomics cases” shall be deemed a reference to the following applications, which are hereby incorporated by reference in their entirety: PCT/US2004/010191, filed Apr. 2, 2004, published as WO2004/092416 on Oct. 28, 2004, our docket Kopchick6.1A-PCT, relating to diabetes-related differential expression in liver, and PCT/US04/17322, filed Jun. 2, 2004 (atty docket Kopchick7A-PCT), related to age-related differential expression.

### BACKGROUND OF THE INVENTION

1. Field of the Invention

The invention relates to various nucleic acid molecules and proteins, and their use in (1) diagnosing hyperinsulinemia and type II diabetes, or conditions associated with their development, and (2) protecting mammals (including humans) against them.

2. Description of the Background Art

Obesity is a major cause of premature morbidity and mortality, especially in the United States, where caloric intake often exceeds energy expenditure. In particular, obesity predisposes individuals to type 2 diabetes mellitus (non-insulin dependent diabetes mellitus, NIDDM), which is characterized by insulin resistance, impaired glucose-stimulated insulin secretion, and pancreatic β-cell dysfunction.

In humans, obesity-induced type 2 diabetes often involves a progression from a normal phenotype through an insulin resistant/hyperinsulinemic state to overt diabetes. These stages are replicated in C57BL/6J mice fed a diet composed of 58% kcal from fat but not those fed a diet with only 14% kcal fat. Mice exposed to the high-fat diet become relatively obese and often develop hyperinsulinemia and diabetes.

### Type 2 Diabetes Mellitus

Diabetes mellitus is a progressive disorder characterized by elevated blood glucose (hyperglycemia). Type 1 diabetes (insulin dependent diabetes mellitus) is characterized by a deficiency of insulin due to the autoimmune destruction of the insulin-producing pancreatic β-cells (1). Type 1 diabetes is relatively rare and beyond the scope of this discussion. In contrast, type 2 diabetes is extremely prevalent. According to recent estimates, type 2 diabetes affects about 5.9% of the population in the United States or 17 million individuals, and is predicted to affect 300 million people worldwide by 2025 (2). The pathogenesis of type 2 diabetes is not completely understood, however, it is closely associated with increased body fat (9).

Type 2 diabetes is characterized by insulin resistance, impaired glucose-stimulated insulin secretion, and β-cell dysfunction. Although the pathogenesis of type 2 diabetes is not completely understood, the “insulin-resistance/islet cell exhaustion” theory suggests that prolonged insulin resistance induces insulin hypersecretion and ultimately pancreatic β-cell failure (3,4). Thus, a period of peripheral hyperinsulinemia precedes the development of overt type 2 diabetes. According to this theory, peripheral insulin resistance stimulates the pancreatic islet cells to hyper-secrete insulin in order to maintain glucose homeostasis. After prolonged hyper-secretion, the islet cells eventually fail and the symptoms of clinical diabetes are manifested (5,6). It is important to recognize that hyperinsulinemia may result from a combination of increased insulin production and decreased utilization by hepatic, muscle and adipose tissue, and that once established, hyperinsulinemia leads to global insulin resistance in all insulin-sensitive tissues.

Obesity is clearly a global epidemic and growing medical problem in the United States (7,9). In 1980, approximately 14.5% of the U.S. population was considered clinically obese whereas currently more than 22.5% of the Americans are clinically obese; and that number continues to rise (7,9). Obesity is defined as an excess of body fat relative to lean body mass (8,9) or by a body-mass-index (BMI; weight divided by the square of height) of 30 kg m-2 or greater (8). By these criteria, about 60 million individuals in the U.S. are obese and related medical spending is ˜$90 billion/year (10). Obesity contributes to premature morbidity and mortality and is associated with the development of type 2 diabetes mellitus (11,12). Obesity-related health risks also include hypertension, dyslipidemia, peripheral vascular disease, and cardiovascular disease, collectively referred to as metabolic syndrome (9).

Obesity precipitates type 2 diabetes, in part, due to an increase in insulin resistance. Indeed, increased peripheral insulin concentrations have been observed in obese individuals (13). Also, the localization or distribution of the fat depots appears to contribute to the risk for type 2 diabetes (14,15). In particular, the accumulation of abdominal and visceral fat depots correlates with type 2 diabetes but the mechanisms controlling depot-specific storage remain unclear (16). Selective overexpression of 11β-hydroxysteroid dehydrogenase type 1 (11βHSD-1) in the adipose tissue of mice results in a disproportionate accumulation of visceral fat and insulin-resistant diabetes. These mice also gain more weight than their non-transgenic littermates when fed a high-fat diet (17).

These observations are important because 11βHSD-1 catalyzes the conversion of glucocorticoids between the active and inactive forms and glucocorticoids inhibit insulin secretion by the pancreas and stimulate gluconeogenesis in the liver.

Adipocytes have depot-specific properties. For example, visceral adipocytes are larger than subcutaneous adipocytes and are more efficient at breaking down stored lipids (7,9). Visceral adipocytes release free fatty acids and other adipocyte-secreted products directly into the liver through the portal vein (18). Consequently, there is a flood of fatty acids into the blood and liver that can either a) inhibit insulin-stimulated glucose uptake into muscle; b) decrease the efficiency of insulin clearance by the liver (7,9); c) increase gluconeogenesis (19); and/or d) potentiate glucose-stimulated insulin secretion (19). Visceral adipocytes were recently shown to express more plasminogen activator inhibitor than subcutaneous adipocytes, providing a possible link between visceral obesity and vascular disease (20,21).

### Consequences of Type 2 Diabetes Mellitus

Chronic hyperglycemia invariably produces macro- and microvascular pathology that in many ways resemble the consequences of aging (22,23). Increased intracellular glucose causes impaired blood flow, increased vascular permeability and excess production of extracellular matrix (ECM) molecules, ultimately resulting in edema, ischemia and hypoxia-induced neovascularization (24). Macrovascular damage results from arterial endothelial cell dysfunction and vascular smooth muscle cell proliferation and increases susceptibility to myocardial infarct (MI, cerebral vascular accident (CVA) and peripheral vascular disease (PVD). Microvascular damage primarily affects the retina, renal glomeruli and peripheral nerves and often causes blindness, end-stage renal disease and neuropathies (24). For example, peripheral neuropathies, characterized by multi-focal demyelination and axonal loss similar to that seen with micro-vascular ischemia, are present in half of the patients with type 2 diabetes, especially those with poor glycemic control, and are the most common cause of non-traumatic amputations (9) Because the risk of developing complications precedes diabetes, it is important to distinguish between the consequences of insulin-resistance and those of hyperglycemia. In addition, identification of individuals who are vulnerable to diabetes-related complications is a critical component of any comprehensive diabetes intervention program.

There are at least four major pathways involved in development of microvascular complications secondary to hyperglycemia (24). The hexosamine pathway is normally used in the biosynthesis of proteoglycans and O-linked glycoproteins. However, excess intracellular glucose can be diverted to this pathway resulting in inappropriately modified proteins or aberrant transcriptional activation of glucose-responsive genes (24).

Overexpression of the rate-limiting enzyme for hexosamine synthesis in cells and transgenic mice leads to insulin resistance, presumably due to diminished translocation of the glucose transporter GLUT4 to the plasma membrane (25). The polyol pathway can be activated at higher concentrations of intracellular glucose. Glucose can be reduced to sorbitol by the NADPH-dependent enzyme, aldose reductase, and sorbitol can be oxidized to fructose by the NAD+-dependent enzyme sorbitol dehydrogenase. Consequently, the cell's redox potential is disrupted making it more vulnerable to oxidative stress (24,26). Intracellular hyperglycemia can also increase the levels of diacylglycerol DAG), a lipid second messenger that activates most protein kinase C(PKC) isoforms. For example, activation of PKC can induce stress-related signal transduction cascades, disrupt osmotic balance through inhibition of Na—K-ATPase, or alter gene expression in vascular and neuronal tissues of diabetic animals (24,27). Finally, the auto-oxidation of intracellular glucose and other reactions generate chemicals (e.g. glyoxyl, methylglyxoyl, 3-deoxyglucosone) that react with the amino groups of intra- and extracellular proteins and lipids to form advanced glycation end-products (AGEs). AGE formation may be especially important in the pathogenesis of diabetic neuropathies (28). AGEs damage cells by interfering with protein function and through inappropriate interactions with cell-surface or nuclear receptors, leading to diverse cellular responses such as the secretion of inflammatory cytokines and generation of reactive oxygen species, altered cell migration and adhesion, or transcription of stress-related genes. The receptor for AGEs (RAGE) is normally expressed at low levels but is upregulated by high concentrations of ligand, such as those observed in the vasculature of diabetics (24,29).

Remarkably, each of these hyperglycemia-induced pathways appears to involve excessive production of superoxide (O2-) by the mitochondrial electron-transport chain (24,30). For example, hyperglycemia increases the production of reactive oxygen species in cultured bovine aortic endothelial cells. This increase was prevented when the cell's ability to utilize electron donors produced by tricarboxylic acid (TCA) cycle, but not glycolysis, was disrupted (30). Abolition of oxidative phosphorylation with pharmacological agents or overexpression of uncoupling protein-1 (UCP1) also prevented the production of free radicals in the presence of excess glucose, as well as the accumulation of AGEs and sorbitol and the activation of PKC (30). Similar experiments indicated that hyperglycemia-induced flux through the hexosamine pathway requires superoxide production and activation of this pathway increases the transcriptional activity of the Sp1 transcription factor by O-glycosylation (31).

### Genes Implicated in the Pathogenesis of Obesity and Type 2 Diabetes Mellitus

DNA microarray analysis was used to compare gene expression (mRNA levels) in white adipose tissue (epididymal fat pads) from different strains of lean and obese (ob-/ob-) mice with varying degrees of hyperglycemia. Obese mice downregulated genes involved in adipocyte differentiation, lipid metabolism and the mitrochondrial electron-transport chain and upregulated genes associated with the cytoskeleton and extracellular matrix and involved in immune system function (32). In addition, 88 genes were identified whose expression correlated with the level of hyperglycemia. For example, the genes encoding the non-receptor protein tyrosine phosphatase PTPK1 and the transcription factor Disheveled decreased as hyperglycemia increased, whereas phosphatase inhibitor-2-like protein and fructose-1,6 bis-phosphatase increased with elevated plasma glucose (32).

Many of the CNS circuits controlling feeding behavior (33) and some of the genes involved in regulating body weight (34) and energy expenditure (35) have been identified. Feeding behavior is influenced by the subjective experience of appetite and the physiological signals that control hunger and satiety (36). A key component of this system is the adipocyte-derived hormone leptin, its receptor in the hypothalamus (37), and the janus-kinase/STAT-3 signal transduction cascade (38). Circulating leptin modulates the release of neuropeptides from the specific neurons in the hypothalamus that either stimulate {e.g. neuropeptide Y (NPY), agouti-related peptide (AgRP), melanin-concentrating hormone (MCH)} or inhibit {e.g. á-melanocyte stimulating hormone (á-MSH), cocaine- and amphetamine-regulated transcript (CART)} feeding behavior. Thus, mice lacking MCH are hypophagic and lean (39), whereas mice lacking the receptor for á-MSH, the melanocortin-4 receptor (MC4-R), are hyperphagic and obese (40). Loss-of-function mutations in the MC4-R gene in humans result in morbid obesity and episodic binge eating (41).

Secretion of leptin is proportional to the body's energy stores in fat depots and it signals to the brain to reduce food intake. Thus, obesity and starvation typically correlate with high and low levels of circulating leptin, respectively (42). However, mice lacking the gene encoding leptin (ob-/ob-) (43) or its receptor (db-/db-) (44) are hyperphagic, obese and diabetic, presumably because they are in a state of perceived starvation. Abnormalities in ob-/ob- mice also include decreases in body temperature, activity, immune function and fertility (37), illustrating the complex relationship between nutritional status, energy homeostasis and reproduction. Although rare, leptin deficiency in humans produces obesity and other metabolic anomalies such as hypogonadism or insulin-resistance, but not diabetes (45,46). Importantly, caloric restriction results in loss of lean and fat mass whereas administration of leptin selectively reduces fit mass. Mice with diet-induced obesity are less sensitive to chronic infusions of exogenous leptin and consequently tend to lose less weight than their lean counterparts (47).

In skeletal muscle and adipose tissue, binding of insulin to its receptor initiates a signal transduction cascade that starts with receptor autophosphorylation on multiple tyrosine residues and tyrosine kinase activation followed by substrate phosphorylation and ultimately translocation of the glucose transporter GLUT4 to the plasma membrane (48). Therefore, targeted disruption of genes in this pathway is a useful way to study insulin resistance, a hallmark of type 2 diabetes. For example, mice with a complete absence of the insulin receptor appear normal at birth but die shortly after in a state of severe hyperinsulinemia, hyperglycemia and ketoacidosis (49). Mice with targeted disruption of the insulin receptor in skeletal muscle have increased adiposity and elevated serum free fatty acids and triglycerides, but are not hyperglycemic, hyperinsulinemic or glucose intolerant. These data suggest that impaired fat metabolism is a consequence of insulin resistance and other tissues are important for glucose disposal (49). Mice with selective deletion of GLUT4 in white and brown adipose tissue have impaired insulin-stimulated glucose uptake in adipose tissue and develop insulin resistance and glucose intolerance (50). It is important to recognize that differential glucose transport may contribute to the differences observed in tissue susceptibility to hyperglycemia-induced tissue damage.

In addition to genes directly involved in insulin signaling and glucose transport, there are many other genes that have been implicated in the pathogenesis of obesity and type 2 diabetes. In particular, several lines of evidence indicate that adipocytes can function as endocrine cells, producing not only fatty acids, but also several bioactive peptides (51,52). A comprehensive discussion about the synthesis, secretion, and regulation of “adipokines” is beyond the scope of this discussion, but several relevant examples will be highlighted.

Two recently discovered hormones, resistin (53) and adiponectin (also called Acrp30, adipocyte complement related protein 30 kDa) are synthesized and secreted by adipocytes and are intimately involved in glucose and lipid metabolism (54). As the name implies, resistin “resists” insulin-stimulated glucose uptake and impairs glucose tolerance. Resistin gene expression is induced during adipocyte differentiation and serum levels are elevated in genetic models of obesity and diabetes (ob-/ob- and db/db) and high-fat diet-induced obesity (53). Adiponectin gene expression is induced during adipocyte differentiation and its secretion is stimulated by insulin. Adiponectin appears to increase tissue sensitivity to insulin. Several missense mutations in the adiponectin gene have been identified in individuals with type 2 diabetes (55). Serum levels of adiponectin are reduced in human and animal models of obesity and insulin resistance. For example, spontaneously occurring obesity and diabetes in rhesus monkeys correspond with a decrease in circulating adiponectin (56). Intraperitoneal administration of recombinant adiponectin in mice inhibits gluconeogenesis and glucose secretion in mouse hepatocytes (57). Intravenous injection of recombinant adiponectin transiently decreased hyperglycemia in ob-/ob- mice and streptozotocin-treated mice without altering serum insulin levels (58). Mice lacking adiponectin have been reported to display moderate insulin resistance, impaired glucose tolerance (59) and increased β-oxidation without insulin resistance and glucose intolerance, despite being fed a high-fat diet (60). The reasons for this discrepancy are unclear, but may involve genetic differences in the strains of mice used in the studies.

Adipsin is a serine protease secreted by adipocytes following differentiation and it may play a role in stimulating triglyceride acylation (61). The expression of adipsin is greatly reduced in many rodent models of diabetes (52). Adipocytes also secrete the inflammatory cytokine tumor necrosis factor (TNF-á) It has recently been shown that TNF-á is expressed at high levels in the adipocytes of obese animals and humans (63,64) and may possibly play a role in insulin resistance (62). Genetically obese mice (ob-/ob-) lacking TNF-á are protected from obesity-induced insulin resistance (65).

The Zucker diabetic fatty (ZDF) rat has defective leptin receptors and develops type 2 diabetes where compensatory insulin hypersecretion is accompanied by an increase in β-cell mass and subsequent β-cell failure is attributed to apoptosis rather than lack of proliferation (66). The β-cells in these animals display altered gene expression of key metabolic enzymes such as glucokinase and ion channels involved in Ca2+-dependent exocytosis (67), supporting the relationship between diabetes, impaired glucose sensitivity and insulin secretion. Mice lacking the insulin receptor substrate IRS-2 are insulin resistant and diabetic but fail to display an increase in β-cell mass, suggesting this molecule is necessary for compensation (68).

Partial pancreatectomy (Px) in rats is another model of type 2 diabetes that leads to a period of β-cell hypertrophy and neogenesis followed by diminished insulin secretion and hyperglycemia, without confounding factors like specific gene mutations or toxin-induced β-cell degeneration. Using this model and quantitative PCR, Weir and colleagues (69-71) have shown that altered 13-cell islet gene expression depends on the magnitude and duration of hyperglycemia. For example, hyperglycemia increased expression of the mRNA encoding the mitochondrial uncoupling protein-2 (UCP-2) and decreased those encoding insulin and the glucose transporter GLUT2 (70). Genes that participate in protection from oxidative stress, such as glutathione peroxidase and heme oxygenase-1, were also upregulated in response to hyperglycemia (69). Hyperglycemia increased expression of the mRNA encoding peroxisome proliferator-activated receptor (PPAR)-alpha and decreased that encoding PPAR-gamma (70). PPARs are ligand-activated transcription factors involved in the regulation of cellular differentiation and proliferation, lipid and glucose homeostasis, and PPARã is the target of the thiazolidinediones (TZDs) class of insulin-sensitizing drugs used to improve glucose-stimulated insulin secretion (72). Interestingly, some of the changes in β-cell gene expression due to short duration hyperglycemia (4 weeks) can be reversed by normalizing blood glucose, whereas those attributed to prolonged hyperglycemia (14 weeks) are less reversible (70,71).

Oxidative metabolism of glucose in the mitochondria generates reducing equivalents that are donated to the electron transport chain and used to produce ATP. The mitochondrial uncoupling proteins (UCPs) disrupt the necessary proton gradient leading to the production of heat instead of ATP. Expression of UCP1 is restricted to brown adipose tissue (BAT) and is critical for adaptive thermogenesis; UCP2 is widely distributed; and UCP3 is expressed primarily in skeletal muscle and BAT (35). The 13-cells of UCP2-deficient mice have increased glucose-stimulated insulin secretion and activation of UCP-2 in leptin-deficient mice correlates with β-cell dysfunction (73). Transgenic mice that overexpress human UCP3 in skeletal muscle consume more calories but weigh less than their non-transgenic littermates. They also have less adipose tissue and lower levels of plasma fatty acids and triglycerides, suggesting a higher rate of 13-oxidation. Finally, these transgenic mice have reduced plasma glucose levels, increased oral glucose tolerance and lower plasma insulin levels (74).

### SUMMARY OF THE INVENTION

Like most complex phenotypes, body weight is regulated by genetic and environmental factors. Nonetheless, in the absence of predisposing genetic influences, obesity results when energy consumption exceeds energy expenditure. Obesity contributes to premature morbidity and mortality and is associated with the development of type 2 diabetes mellitus. We believe that the physiological consequences of obesity and type 2 diabetes correspond to distinct protein profiles indicative of stage and severity of disease progression.

Since the risk of developing complications precedes diabetes, it is desirable to distinguish between the consequences of insulin-resistance and those of hyperglycemia Results from our experiments have been used to identify factors that predispose individuals to or protect them from diabetes-related complications. The ability to identify individuals vulnerable to complications will be invaluable to comprehensive diabetes intervention programs.

Our approach is proteomics-based (98); it directly identifies differentially expressed proteins with the aid of two-dimensional gel-electrophoresis and mass spectrometry. Unlike genomics-based methods, it can detect differential expression of post- or co-translationally modified proteins(99,100). Proteomic analysis has been used to detect disease associated polymorphisms in mouse brain (101).

Mice reared on a high-fat diet are relatively obese compared to age-matched controls fed a normal diet, and display progressive deterioration in glucose homeostasis. Consequently, proteins which are expressed at higher or lower levels in such mice, as compared to those on a normal (low fat) diet, are likely to be involved in the disease progression.

Mice reared on each diet were monitored at regular intervals for evidence of obesity and diabetes (i.e. weight; glucose and insulin levels). To identify targets for diagnostic and therapeutic agents, the physiological parameters were correlated with the relative abundance of proteins that are differentially expressed or modified as a consequence of obesity and diabetes.

The insulin-sensitive tissues (i.e. liver, skeletal muscle, white adipose, pancreas) and tissues susceptible to diabetes-related complications (i.e. kidney, heart, brain) contain proteins whose timing and pattern of expression are believed to correlate with the stage and severity of obesity and diabetes. We believe that there are significant differences in the way each tissue responds to diet-induced obesity and diabetes.

Serum and skin are also believed to contain such proteins. Serum in any event is clinically relevant, has established age—and diabetes—related biomarkers, and is readily accessible. Skin is considered worth studying because it is readily available and can be obtained using a minimally invasive punch biopsy that might also extract the associated endothelial-rich vascular tissue.

The term “tissue” may refer to tissues which are part of an organ (e.g., heart tissue) or tissues which aren't (e.g., muscle tissues, subcutaneous tissue, etc.). The term “tissue”, as used herein, is intended to include serum and skin. Should it be desirable to refer to tissues other than serum, the term “solid tissue” will be used.

For the purpose of the instant application, the tissue of interest was serum. That is, the serum samples were compared to identify mouse proteins which were expressed at different levels in serum from normal, hyperinsulinemic and/or diabetic mice of a particular age, and the identification of differentially expressed mouse proteins in Master Tables 101-103 is strictly with respect to differential expression in serum However, the findings with respect to serum may be compared with the differential expression findings vis-a-vis other tissues.

Insulin-sensitive tissues, tissues susceptible to hyperglycemia-related damage, and serum, were harvested from the experimental animals at different stages of disease severity. In parallel, tissue were prepared for proteomic analysis.

Proteins can be isolated from distinct subcellular fractions by differential gradient ultracentrifugation or homogenized as total protein lysates and then resolved by two-dimensional gel-electrophoresis. The relative abundance of each protein were determined by densitometry and differentially expressed or modified proteins were excised from the gels and prepared for mass spectrometry. Peak intensity spectra were used to predict the peptide fragments found in each sample. When necessary, a protein's identity was confirmed by western blot analysis and its pattern of expression was determined by immunocytochemistry.

For analysis, each protein “spot” was assigned an intensity corresponding to its relative pixel density and a grid location based on its location in the gel. Proteins were selected for further analysis if their relative abundance is altered as a consequence of obesity and diabetes. Protein “spots” were manually excised from the gels and prepared for automated mass spectrometry analysis. The peptide mass fingerprint data were thoroughly analyzed to determine the confidence of the predictions.

We can enrich a fraction for a particular protein to compensate for its relative lack of abundance and use immunohistochemistry to assess cellular localization. One enormous advantage of proteomics is that isolation of enough protein “spots” is sufficient for antibody or crystal production. Non-denaturing IEF experiments could even isolate a protein that maintains biological activity.

These experiments identify mouse proteins (usually tissue-specific) whose timing and pattern of expression correlates with the stage and severity of obesity and diabetes.

The corresponding mouse protein profiles provide insight about the control of functional mouse protein networks and reveal novel targets for the diagnosis and treatment of type 2 diabetes. By a “profile”, we mean the state of the proteome at a particular stage of the disease progression (normal to hyperinsulinemic to diabetic; or normal to overweight to obese; these two progressions are related but not necessarily synchronized) and, more particularly, the elements of the proteome which have changed relative to the other stage(s).

We deliberately did not limit our analysis to a particular network, such as networks of proteins involved in insulin signaling or in protecting cells from oxidative stress. Rather our systematic approach with flexible inclusion criteria avoids selections based on criteria that are too stringent or not stringent enough, and will allow us distinguish robust and weak signals under different circumstances, such that we may identify novel networks that had previously been obscured or overlooked. Whether by complex algorithms or visual inspection, once the appropriate targets emerge from the tissue-specific protein profiles, we take a more stochastic approach to identify associated proteins using a variety of techniques including co-immunoprecipitation and affinity tagged-mass spectrometry.

Corresponding human proteins can be identified by searching human protein sequence databases for homologous proteins. The sequences in the protein databases are determined either by directly sequencing the protein or, more commonly, by sequencing a DNA, and then determining the translated amino acid sequence in accordance with the Genetic Code. All of the mouse sequences in the mouse polypeptide database are referred to herein as “mouse proteins” regardless of whether they are in fact full length sequences (i.e., encoded by a full-length DNA). Likewise, the human sequences in the human polypeptide database are referred to as “human proteins”.

Mouse proteins which were differentially expressed (normal vs. hyperinsulinemic, hyperinsulinemic vs. diabetic, or normal vs. diabetic), as measured by fasting serum insulin and glucose levels were identified.

One may further take into account whether the subject is normoinsulinemic or hyperinsulinemic at the time of the assay. If the subject is non-diabetic and normoinsulinemic, we are especially interested in the “favorable” and “unfavorable” proteins corresponding to mouse proteins differentially expressed in hyperinsulinemic vs. normal tissue. If the subject is already hyperinsulinemic, yet non-diabetic, we are especially interested in the “favorable” and “unfavorable” proteins corresponding to mouse proteins differentially expressed in type H diabetic vs. hyperinsulinemic tissue.

Since the progression is from normal to hyperinsulinemic, and thence from hyperinsulinemic to type II diabetic, one may define mammalian subjects as being more favored or less favored, with normal subjects being more favored than hyperinsulinemic subjects, and hyperinsulinemic subjects being more favored than type II diabetic subjects. The subjects' state may then be correlated with their gene expression activity.

The terms “normal” and “control” are used interchangeably in this specification, unless expressly stated otherwise. The control or normal subject is a mouse which is normal vis-a-vis fasting insulin and fasting glucose levels. The term “normal”, as used herein, means normal relative to those parameters, and does not necessitate that the mouse be normal in every respect.

A mouse protein is said to have exhibited a favorable behavior if, for a particular mouse age of observation, its average level of expression in mice which are in a more favored state is higher than that in mice which are in a less favored state. A mouse protein is said to have exhibited an unfavorable behavior if, for a particular mouse age of observation, its average level of expression in mice which are in a more favored state is lower than that in mice which are in a less favored state.

When we observe the mice at several different ages, it is possible for their expression behavior to vary from time point to time point. For a given comparison of subjects, e.g., normal vs. hyperinsulinemic, we classify the mouse protein as favorable or unfavorable on the basis of the direction of the largest expression change, and it is the magnitude of this largest expression change, expressed as a ratio of greater to lesser, which is set forth in the Master Table 1 data for that mouse protein. Thus, if at 2 weeks, there was a 3-fold favorable behavior, and at 8 weeks, there was a 4-fold unfavorable behavior, and at all other time points, the behavior was weaker than 3-fold, the mouse protein would be classified as an unfavorable protein with respect to the subject comparison in question.

It will be appreciated that it may be that if the mouse protein were observed at an age other than one of the ages noted in the Examples, we would have observed a still stronger differential expression behavior. Nonetheless, we must classify the mouse proteins on the basis of the behavior which we actually observed, not the behavior which might have been observed at some other age.

We are particularly interested in mouse proteins which exhibit strongly favorable or unfavorable differential expression behaviors. A behavior is considered strong if the ratio of the higher level to the lower level is at least two-fold.

However, a mouse protein may still be identified as favorable or unfavorable even if none of its observed behaviors are substantial as defined above. In general, we consider the consistency of its behaviors (that is, are all or most of the differential expression behaviors at different ages in the same direction, e.g., hyperinsulinemic higher than control), the magnitude of the behaviors (higher the better), and the expression behavior of structurally or functionally related mouse proteins (a mouse protein is more likely to be identified as favorable on the basis of a weakly favorable behavior if it is related to other mouse proteins which exhibited favorable, especially strongly favorable, behavior). If we considered a mouse protein with only weak differential expression behavior to be worthy of consideration on the basis of these criteria, then we listed it in Master Table 1 in the appropriate subtable.

Preferably, the differential behavior observed is both strong and consistent. Preferably, if related mouse proteins were tested, they exhibit the same direction of differential expression behavior.

A mouse protein which was more strongly expressed in hyperinsulinemic tissue than in either normal or type II diabetic tissue (i.e., C<HI, HI>D) will be deemed both “unfavorable”, by virtue of the control:hyperinsulinemic comparison, and “favorable”, by virtue of the hyperinsulinemic:diabetic comparison. This is one of several possible “mixed” expression patterns.

Thus, we can subdivide the “favorables” into wholly and partially favorables. Likewise, we can subdivide the unfavorables into wholly and partially unfavorables. The proteins with “mixed” expression patterns are, by definition, both partially favorable and partially unfavorable. In general, use of the wholly favorable or wholly unfavorable proteins is preferred to use of the partially favorable or partially unfavorable ones.

It is evident from the foregoing that mixed proteins are those exhibiting a combination of favorable and unfavorable behavior. A mixed protein can be used as would a favorable protein if its favorable behavior outweighs the unfavorable. It can be used as would an unfavorable protein if its unfavorable behavior outweighs the favorable. Preferably, they are used in conjunction with other agents that affect their balance of favorable and unfavorable behavior. Use of mixed proteins is, in general, less desirable than use of purely favorable or purely unfavorable proteins, but it is not excluded.

It should be noted that a mouse protein is classified on the basis of the strongest C-HI behavior among the ages tested, the strongest HI-D behavior among the ages tested, and the strongest C-D behavior among the ages tested. If at least one of these three behaviors is significantly favorable, and none of the others of these three behaviors is significantly unfavorable, the mouse protein will be classified as wholly favorable and listed in subtable 1A of Master Table 1. However, that does not mean that it may not have exhibited a weaker but unfavorable expression behavior at some tested age.

The “favorable”, “unfavorable” and “mixed” mouse proteins of the present invention include the mouse database proteins listed in the Master Table.

The mouse proteins of interest also include mouse proteins which, while not listed in the table, correspond to (i.e., homologous to, i.e., which could be aligned in a statistically significant manner to) such mouse proteins or genes, and mouse proteins which are at least substantially identical or conservatively identical to the listed mouse proteins.

Related proteins were identified by searching a database comprising human proteins for sequences corresponding to (i.e., homologous to, i.e., which could be aligned in a statistically significant manner to) the mouse protein. More than one human protein may be identified as corresponding to a particular mouse protein.

Note that the term “human proteins” are used in a manner analogous to that already discussed in the case of “mouse proteins”.

As used herein, the term “corresponding” does not mean identical, but rather implies the existence of a statistically significant sequence similarity, such as one sufficient to qualify the human protein as a homologous proteinas defined below. The greater the degree of relationship as thus defined (i.e., by the statistical significance of the alignment, measured by an E value), the more close the correspondence.

In general, the human proteins which most closely correspond, directly or indirectly, to the mouse proteins are preferred, such as the one(s) with the highest, top two highest, top three highest, top four highest, top five highest, and top ten highest homologies (lowest E values) for the BlastP alignment to a particular mouse protein. The human proteins deemed to correspond to our mouse proteins are identified in the Master Tables.

Note that it is possible to identify homologous full-length human proteins, if they are present in the database, even if the query mouse protein sequence is not a full-length sequence.

If there is no homologous full-length human protein in the database, but there is a partial one, the latter may nonetheless be useful. For example, a partial protein may still have biological activity, and a molecule which binds the partial protein may also bind the full-length protein so as to antagonize a biological activity of the full-length protein.

The protein sequences may of course also be used in the design of probes intended to identify the full length gene or protein sequence.

For the sake of convenience, we refer to a human protein as favorable if (1) it is listed in Master Table 1 as corresponding to a favorable mouse protein, or (2) it is at least substantially identical or conservatively identical to a listed protein per (1). We define a human protein as unfavorable in an analogous manner. We may further identify a human protein as being wholly favorable (see mouse proteins of subtable 1A, wholly unfavorable (see mouse proteins of subtable 1B), or mixed, i.e., both partially favorable and partially unfavorable(see mouse proteins of subtable 1C).

However, it should be noted that this classification is not based on the direct study of the expression of the human protein of course, the human proteins of ultimate interest will be the ones whose change in level of expression is, in fact, correlated, directly or inversely, with the change of state (normal, hyperinsulinemic, diabetic) of the subject.

After identifying related human proteins, one may formulate agents useful in screening humans at risk for progression toward hyperinsulinemia or toward type II diabetes, or protecting humans at risk thereof from progression from a normoinsulinemic state to a hyperinsulinemic state, or from either to a type II diabetic state.

Agents which bind the “favorable” and “unfavorable” human proteins (e.g., an antibody vs. a human protein identified as corresponding to a favorable or unfavorable mouse protein) may be used to evaluate whether a human subject is at increased or decreased risk for progression toward type II diabetes. A subject with one or more elevated “unfavorable” and/or one or more depressed “favorable” proteins is at increased risk, and one with one or more elevated “favorable” and/or one or more depressed “unfavorable” proteins is at decreased risk.

One may further take into account whether the subject is normoinsulinemic or hyperinsulinemic at the time of the assay. If the subject is non-diabetic and normoinsulinemic, we are especially interested in the “favorable” and “unfavorable” proteins corresponding to mouse proteins differentially expressed in hyperinsulinemic vs. normal tissue. If the subject is already hyperinsulinemic, yet non-diabetic, we are especially interested in the “favorable” and “unfavorable” proteins corresponding to mouse proteins differentially expressed in type II diabetic vs. hyperinsulinemic tissue.

The assay may be used as a preliminary screening assay to select subjects for further analysis, or as a formal diagnostic assay.

The identification of the related proteins may also be useful in protecting humans against these disorders.

Applicants contemplate, as a result of the identification of favorable mouse proteins and of corresponding human proteins, the use of:

(1) Human proteins corresponding to favorable mouse proteins (and of the mouse proteins, or other corresponding nonhuman proteins, if biologically active in humans), to protect against the disorder(s);

(2) DNAs encoding those proteins to express the latter in vitro, the proteins being subsequently administered to a patient;

(3) Such DNAs in gene therapy to express those human proteins in vivo;

(4) Such proteins in diagnostic agents, in assays to measure progression toward hyperinsulinemia or type II diabetes, or protection against the disorder(s), or to estimate related end organ damage such as kidney damage; and

(5) DNAs which hybridize to the human mRNAs encoding those human proteins (or to corresponding cDNAs), in diagnostic agents, for the purposes set forth in (4) above.

Moreover, Applicants contemplate, as a result of the identification of unfavorable mouse proteins and of corresponding human proteins, the use of:

(1) the mouse or human proteins in assays to determine whether a substance binds to (and hence may neutralize) the protein; and

(2) the neutralizing substance to protect against the disorder(s).

(3) the corresponding mouse or human proteins, in diagnostic agents, competing with sample protein for binding to another diagnostic agent, to measure progression toward hyperinsulinemia or type II diabetes, or protection against the disorder(s), or to estimate related end organ damage such as kidney damage;

(4) DNAs encoding such proteins to express the latter in vitro;

(5) Nucleic acids which hybridize to mRNAs encoding the “unfavorable” human proteins, as antisense molecules to inhibit expression of the latter;

(6) such hybridizing nucleic acids, in diagnostic reagents, to bind such mRNA and determine mRNA levels, and hence for the purposes set forth in (3) above; and

(7) substances which bind the mouse or human protein in diagnostic agents, for the purposes recited in (3) above.

Our animal models of hyperinsulinemia and diabetes are also obese. It is possible that the proteins found to be favorable act indirectly by inhibiting obesity. Likewise, it is possible that the proteins found to be unfavorable act indirectly by accentuating obesity. Consequently, it is

within the compass of the present invention to use the favorable proteins, or to use antagonists of the unfavorable proteins, to protect against obesity, as well as against sequelae of obesity such as hyperinsulinemia and diabetes.

Since type II diabetes is an age-related disease, the agents of the present invention may be used in conjunction with known anti-aging or anti-age-related disease agents. It is of particular interest to use the agents of the present invention in conjunction with an agent disclosed in one of the related applications cited above, in particular, an antagonist to CIDE-A, the latter having been taught in U.S. Ser. No. 60/474,606, filed Jun. 2, 2003 (atty docket Kopchick7), and PCT/US04/17322, filed Jun. 2, 2004 (atty docket Kopchick7A-PCT), hereby incorporated by reference in their entirety.

### DETAILED DESCRIPTION OF THE PREFERRED EMBODIMENTS OF THE INVENTION

### Full-Length vs. Partial Length Genes/Proteins

A “full length” gene is here defined as (1) a naturally occurring DNA sequence which begins with an initiation codon (almost always the Met codon, ATG), and ends with a stop codon in phase with said initiation codon (when introns, if any, are ignored), and thereby encodes a naturally occurring polypeptide with biological activity, or a naturally occurring precursor thereof, or (2) a synthetic DNA sequence which encodes the same polypeptide as that which is encoded by (1). The gene may, but need not, include introns.

A “full-length” protein is here defined as a naturally occurring protein encoded by a full-length gene, or a protein derived naturally by post-translational modification of such a protein. Thus, it includes mature proteins, proproteins, preproteins and preproproteins. It also includes substitution and extension mutants of such naturally occurring proteins.

Some protein “spots” will represent post-translational modifications of the same protein while others may represent heterogeneity due to genetic polymorphisms. For example, 2D gels often reveal a “charge” train representing a difference in phosphorylation states of the same protein.

### Subjects

A mouse is considered to be a diabetic subject if, regardless of its fasting plasma insulin level, it has a fasting plasma glucose level of at least 190 mg/dL. A mouse is considered to be a hyperinsulinemic subject if its fasting plasma insulin level is at least 0.67 ng/mL and it does not qualify as a diabetic subject. A mouse is considered to be “normal” if it is neither diabetic nor hyperinsulinemic. Thus, normality is defined in a very limited manner.

A mouse is considered “obese” if its weight is at least 15% in excess of the mean weight for mice of its age and sex. A mouse which does not satisfy this standard may be characterized as “non-obese”, the term “normal” being reserved for use in reference to glucose and insulin levels as previously described.

A human is considered a diabetic subject if, regardless of his or her fasting plasma insulin level, the fasting plasma glucose level is at least 126 mg/dL. A human is considered a hyperinsulinemic-subject if the fasting plasma insulin level is more than 26 micro International Units/mL

(it is believed that this is equivalent to 1.08 ng/mL), and does not qualify as a diabetic subject. A human is considered to be “normal” if it is neither diabetic nor hyperinsulinemic. Thus, normality is defined in a very limited manner.

A human is considered “obese” if the body mass index (BMI) (weight divided by height squared) is at least 30 kg/m². A human who does not satisfy this standard may be characterized as “non-obese”, the term “normal” being reserved for use in reference to glucose and insulin levels as previously described.

A human is considered overweight if the BMI is at least 25 kg/m². Thus, we define overweight to include obese individuals, consistent with the recommendations of the National Institute of Diabetes and Digestive and Kidney Diseases(NIDDK). A human who does not satisfy this standard may be characterized as “non-overweight.”

According to the Report of the Expert Committee on the Diagnosis and Classification of Diabetes Mellitus, Diabetes Care 20: 1183-97 (1997), the following are risk factors for diabetes type II: older (e.g., at least 45; see below); excessive-weight (see below); first-degree relative with diabetes mellitus; member of high risk ethnic group (black, Hispanic, Native American, Asian); history of gestational diabetes mellitus or delivering a baby weighing more than 9 pounds (4.032 kg); hypertensive (>140/90 mm Hg); HDL cholesterol level>35 mg/dL (0.90 mmol/L); and triglyceride level >=250 mg/dL (2.83 mmol/L). Hence, in a preferred embodiment, the diagnostic and protective methods of the present invention are applied to human subjects exhibiting one or more of the aforementioned risk factors. Likewise, in a preferred embodiment, they are applied to human subjects who, while not diabetic, exhibit impaired glucose homeostasis (110 to <126 mg/dL).

The risk of diabetes increases with age. Hence, in successive preferred embodiments, the age of the subjects is at least 45, at least 50, at least 55, at least 60, at least 65, at least 70, and at least 75.

With regard to excessive weight, NIDDK says that “The relative risk of diabetes increases by approximately 25 percent for each additional unit of BMI over 22.” Hence, in successive preferred embodiments, the BMIs of the human subjects is at least 23, at least 24, at least 25 (i.e., overweight by our criterion), at least 26, at least 27, at least 28, at least 29, at least 30 (i.e., obese), at least 31, at least 32, at least 33, at least 34, at least 35, at least 36, at least 37, at least 38, at least 39, at least 40, or over 40.

### Antagonists

If we have indicated that an antagonist of a protein or other molecule is useful, then such an antagonist may be obtained by preparing a combinatorial library, as described below, of potential antagonists, and screening the library members for binding to the protein or other molecule in question. The binding members may then be further screened for the ability to antagonize the biological activity of the target. The antagonists may be used therapeutically, or, in suitably labeled or immobilized form, diagnostically.

Substances known to interact with an identified mouse or human protein (e.g., agonists, antagonists, substrates, receptors, second messengers, regulators, and so forth), and binding molecules which bind them, are also of utility. Such binding molecules can likewise be identified by screening a combinatorial library.

### Identification of Differentially Expressed Mouse Proteins

The mass spectrum (MS) of the peptide mixture resulting from the digestion of a protein by an enzyme provides a “fingerprint” by which the protein can be identified, provided that the protein has a sequence which is published in a sequence database. In essence, for each database protein, the identification software determines what fragments would be generated from that database protein if it were subjected to the same treatment as was the recovered protein, and calculates their masses. The program also determines how good a fit there is between the set of mass peaks observed for the actual protein, and the set of mass peaks generated in silico for each database protein.

Tandem mass spectrometry deliberately induces fragmentation of a precursor ion and then analyzes the resulting fragments. Since the precursor ion is itself derived from one of the peptide fragments of the original protein, the analysis is called MS/MS.

For each gel spot, the recovered protein was identified, based on the mass spectrogram of its digest, using one or more of the following analytical tools: Mascot MS, Mascot MS/MS (for up to four fragments of the protein), and MS-FIT. Each of these tools generates a match score which is a measure (although not the only conceivable one) of the degree of fit. The score can take into account, e.g., the apparent molecular weight of the peak, the mass difference between the observed and predicted peaks, and whether the matching predicted fragment has any missed cleavages. The match score is given in the form of the Probability-Based MOWSE score.

In a preferred embodiment, the human proteins of Master Table 1 are those which are homologous to the mouse proteins with the better match scores. The higher the score, the higher the number of masses matched, and/or the higher the quality of the peak match.

The human protein of interest is preferably homologous to a mouse protein for which the Mascot-MS match score is at least 64, more preferably at least 75, even more preferably at least 100.

If Mascot MS/MS is performed, then the human protein of Master Table 1 is preferably homologous to a mouse protein for which the Mascot MS/MS score for at least one fragment is at least 24, more preferably at least 27, even more preferably at least 50.

This is especially desirable if the mouse protein does not satisfy the Mascot MS match score desideratum stated above.

The E value of the top scoring mouse database protein will depend on whether the recovered mouse protein is actually in the database, the accuracy of the database sequence (inaccuracies will reduce the score and hence the E value for that score), and on the specified mass tolerance (the higher the tolerance, the more likely it is that a database protein will match some masses by chance alone).

In the case of Mascot MS and Mascot MS/MS, the significance of the match score is stated as an E value. The E value is the number of times that an alignment scoring at least as good as the one observed would occur in the course of the database search (given the number of database sequences) through chance alone. Consequently, the lower the E value, the more significant the result.

In the case of MS-FIT, this application provides only a MOWSE match score. The MOWSE score is based on the scoring system described in Pappin et al., Current Biology, 3(6): 327 (1993). The higher the MOWSE score, the better the fit.

Preferably, for each database mouse protein of Master Table 1, at least one of the following desiderata applies:

(1) the Mascot MS E value is not more than 0.05;
(2) the Mascot MS/MS E value for at least one fragment is not more than 0.05;
(3) the MS-FIT MOWSE score is at least 10.
More preferably, two or all three of these desiderata apply.

It is also desirable that these desiderata be exceeded. Thus, the Mascot MS E value is more preferably less than e-3, even more preferably less than e-4, still more preferably less than e-5, most preferably less than e-6.

Likewise, the Mascot MS/MS E value is more preferably less than e-3, even more preferably less than e-4, still more preferably less than e-5, most preferably less than e-6.

Finally, the MS-FIT MOWSE score is more preferably more than 100, even more preferably more than 1000, still more preferably more than 10,000, most preferably more than 100,000.

It would be tedious to enumerate all the possible combinations of preferences for the three types of scores, nonetheless, each possible combination is a contemplated preferred embodiment.

Consideration can further be given to the following factors:

comparison of the apparent molecular weight of the recovered protein to the calculated molecular weight of the database protein (it is desirable that the analyzed protein have a molecular weight not more than 10% greater than the database protein; more leniency is appropriate when the molecular weight is lower as the database protein is often the least processed form of the protein and the analyzed protein may be a cleavage product of the database protein);
comparison of the apparent pI of the recovered protein to the calculated pI of the database protein (preferably within 2.0 units, more preferably within 1.5 units, even more preferably within 1.0 units, most preferably within 0.5 units);
the ratio of the number of matched peaks to the number of total peaks (preferably at least 1:10, more preferably at least 1:5);
the percentage of the database protein which is covered by the matched peaks (preferably at leas 10%, more preferably at least 20%, even more preferably at least 30%);
the distribution of the matched peaks within the database protein.
When the apparent molecular weight of the protein is smaller than the calculated molecular weight of the database protein, this may be because the isolated protein corresponds to a fragment of the database protein. If the matched peptide fragments (actual vs. predicted) can be localized to one region of the database protein, e.g., the C-terminal, and that region is similar in molecular weight to the observed molecular weight, then this would support the hypothesis that the isolated protein was a fragment of the database protein.

### Corresponding (Homologous) Proteins

A human protein can be said to be identifiable as corresponding (homologous) to a mouse protein if it can be aligned by BlastP to the mouse protein, where any alignment by BlastP is in accordance with the default parameters set forth below, and the expected value (E) of each alignment (the probability that such an alignment would have occurred by chance alone) is less than e-10. (Note that because this is a negative exponent, a value such as e-50 is less than e-10.). Preferably the E value is less than e-50, more preferably less than e-60, still more preferably less than e-70, even more preferably less than e-80, considerably more preferably less than e-90, and most preferably less than e-100. Desirably, it is true for two or even all three of these conditions.

In constructing Master table 1, we generally used a BlastP (mouse protein vs. human protein) alignment E value cutoff of e-50. However, if there were no human proteins with that good an alignment to the mouse protein in question, or if there were other reasons for including a particular human protein (e.g., a known functionality supportive of the observed differential cognate mouse protein expression), then a human protein with a score worse (i.e., higher) than e-50 may appear in Master Table 1.

If the identified mouse protein corresponds to an EST, or other mouse DNA which is not a full-length mouse gene, a longer (possibly full length) mouse gene may be identified by a BlastN search of the mouse DNA database, using the mouse DNA exactly corresponding to the identified mouse protein as a query sequence. The mouse protein encoded by the longer mouse database DNA may then be deduced using the genetic code, and itself used in a BlastP search of human proteins.

Master table 1 assembles a list of human protein corresponding to each of the mouse proteins identified herein. These human proteins form a set and can be given a percentile rank, with respect to E value, within that set. The human proteins of the present invention preferably are those scorers with a percentile rank of at least 50%, more preferably at least 60%, still more preferably at least 70%, even more preferably at least 80%, and most preferably at least 90%.

For each mouse protein in Master Table 1, there is a particular human protein which provides the best alignment match as measured by BlastP, i.e., the human protein with the best score (lowest e-value). These human proteins form a subset of the set above and can be given a percentile rank within that subset, e.g., the human proteins with scores in the top 10% of that subset have a percentile rank of 90% or higher. The human proteins of the present invention preferably are those best scorer subset proteins with a percentile rank within the subset of at least 50%, more preferably at least 60%, still more preferably at least 70%, even more preferably at least 80%, and most preferably at least 90%.

BlastP can report a very low expected value as A0.0″. This does not truly mean that the expected value is exactly zero (since any alignment could occur by chance), but merely that it is so infinitesimal that it is not reported. The documentation does not state the cutoff value, but alignments with explicit E values as low as e-178 (624 bits) have been reported as nonzero values, while a score of 636 bits was reported as A0.0″.

Functionally homologous human proteins are also of interest. A human protein may be said to be functionally homologous to the mouse protein if the human protein has at least one biological activity in common with the mouse protein encoded by said mouse protein.

The human proteins of interest also include those that are substantially and/or conservatively identical (as defined below) to the homologous and/or functionally homologous human proteins defined above.

### Database Searching

Once a known human protein is identified, it may be used in further BlastP searches to identify other human proteins.

Searches may also take cognizance, intermediately, of known proteins other than mouse or human ones, e.g., use the mouse sequence to identify a known rat sequence and then the rat sequence to identify a human one.

If we have identified a mouse protein which appears similar to a human protein, then that human protein may be used (especially in humans) for purposes analogous to the proposed use of the mouse protein in mice.

In determining whether the disclosed proteins have significant similarities to known proteins, one would generally use the disclosed protein as a query sequence in a search of a sequence database. The results of several such searches are set forth in the Examples. Such results are dependent, to some degree, on the search parameters. Preferred parameters are set forth in Example 1. The results are also dependent on the content of the database. While the raw similarity score of a particular target (database) sequence will not vary with content (as long as it remains in the database), its informational value (in bits), expected value, and relative ranking can change. Generally speaking, the changes are small.

It will be appreciated that the protein databases keep growing. Hence a later search may identify high scoring target sequences which were not uncovered by an earlier search because the target sequences were not previously part of a database.

Hence, in a preferred embodiment, the cognate proteins include not only those set forth in the examples, but those which would have been highly ranked (top ten, more preferably top three, even more preferably top two, most preferably the top one) in a search run with the same parameters on the date of filing of this application.

### Degree of Differential Expression

The degree of differential expression may be expressed as the ratio of the higher expression level to the lower expression level. Preferably, this is at least 2-fold, and more preferably, it is higher, such as at least 3-fold, at least 4-fold, at least 5-fold, at least 6-fold, at least 7-fold, at least 8-fold, at least 9-fold, or at least 10-fold. Most preferably, the human protein of interest corresponds to a mouse protein for which the degree of differential expression places it among the top 10% of the mouse proteins in the appropriate subtable.

### Relevance of Favorable and Unfavorable Proteins

If a protein is down-regulated in more favored mammals, or up-regulated in less favored mammals, (i.e., an “unfavorable protein”) then several therapeutic utilities are apparent.

First, an agent which is an antagonist of the unfavorable protein, or of a downstream product through which its activity is manifested (e.g., a signaling intermediate), may be used to inhibit its activity.

This antagonist could be an antibody, a peptide, a peptoid, a nucleic acid, a peptide nucleic acid (PNA) oligomer, a small organic molecule of a kind for which a combinatorial library exists (e.g., a benzodiazepine), etc. An antagonist is simply a binding molecule which, by binding, reduces or abolishes the undesired activity of its target. The antagonist, if not an oligomeric molecule, is preferably less than 1000 daltons, more preferably less than 500 daltons.

Secondly, an agent which degrades, or abets the degradation of the protein or of a downstream product which mediates its activity (e.g., a signaling intermediate), may be used to curb the effective period of activity of the protein.

Thirdly, an agent which down-regulates expression of the gene may be used to reduce levels of the corresponding protein and thereby inhibit further damage. This agent could inhibit transcription of the gene in the subject, or translation of the corresponding messenger RNA. Possible inhibitors of transcription and translation include antisense molecules and repressor molecules. The agent could also inhibit a post-translational modification (e.g., glycosylation, phosphorylation, cleavage, GPI attachment) required for activity, or post-translationally modify the protein so as to inactivate it. Or it could be an agent which down- or up-regulated a positive or negative regulatory gene, respectively.

While the design of an antisense molecule would require knowledge of the sequence of the gene (to inhibit transcription) or of the mRNA (to inhibit translation), it is possible to identify a repressor molecule without knowing the identity of the sequence to which it binds.

Likewise, several assay utilities are apparent. An assay can be used to determine the level of the unfavorable protein or the corresponding mRNA in a sample. Such an assay could be for quality control purposes, if the sample were from in vitro production of the protein. If the sample is from a subject, this can, if desired, be correlated with prognostic information and used to diagnose the present or future state of the subject, making the assay a diagnostic assay. Elevated levels are indicative of progression, or propensity to progression, to a less favored state, and clinicians may take appropriate preventative, curative or ameliorative action.

First, the unfavorable protein, or a suitable fragment thereof, may be used in labeled or immobilized form as an assay reagent, in the assaying of a sample to determine the level of the protein. (It would compete with the sample protein.) Likewise, a substance which binds the unfavorable protein may be used in labeled or immobilized form as an assay reagent to label or capture the sample protein.

Secondly, if the gene encoding the protein is known, the complementary strand of the gene, or the corresponding cDNA, or a specifically hybridizing fragment of the gene or cDNA, may be used in labeled form as a hybridization probe to detect messenger RNA (or its cDNA) and thereby monitor the level of expression of the gene in a subject.

If a protein is up-regulated in more favored mammals, or down-regulated in less favored animals then the utilities are converse to those stated above.

First, the protein may be administered for therapeutic purposes. Likewise, an agent which is an agonist of the protein, or of a downstream product through which its activity (of inhibition of progression to a less favored state) is manifested, or of a signaling intermediate may be used to foster its activity.

Secondly, an agent which inhibits the degradation of that protein or of a downstream product or of a signaling intermediate may be used to increase the effective period of activity of the protein.

Thirdly, an expression vector comprising an expressible DNA encoding the favorable protein may be administered to the subject (“gene therapy”) to increase the level of expression of the protein in vivo. It could be a vector which carries a copy of the gene, but which expresses the gene at higher levels than does the endogenous expression system.

Fourthly, an agent which up-regulates expression of the gene encoding the favorable protein may be used to increase levels of that protein and thereby inhibit further progression to a less favored state. It could be an agent which up- or down-regulates a positive or negative regulatory gene. Or it could be an agent which modifies in situ the regulatory sequence of the endogenous gene by homologous recombination.

Likewise, assay (including diagnostic) utilities for the favorable protein, and related nucleic acids, exist.

First, the protein, or a binding molecule therefor, may be used, preferably in labeled or immobilized form, as an assay reagent in an assay for said protein product or downstream product. Depressed levels of the favorable protein are indicative of damage, or possibly of a propensity to damage, and clinicians may take appropriate preventative, curative or ameliorative action.

Second, the complementary strand of the corresponding gene, or its cDNA, or a specifically hybridizing fragment of the gene or cDNA, may be used in labeled form as a hybridization probe to detect messenger RNA and thereby monitor the level of expression of the gene in a subject.

### Mutant Proteins

The present invention also contemplates mutant proteins (peptides) which are substantially identical (as defined below) to the parental protein (peptide). In general, the fewer the mutations, the more likely the mutant protein is to retain the activity of the parental protein. The effect of mutations is usually (but not always) additive. Certain individual mutations are more likely to be tolerated than others.

A protein is more likely to tolerate a mutation which:

(a) is an amino acid substitution rather than an insertion or deletion of one or more amino acids;

(b) is an insertion or deletion of one or more amino acids at either terminus, rather than internally, or, if internal, at a domain boundary, or a loop or turn, rather than in an alpha helix or beta strand;

(c) affects a surface amino acid residue rather than an interior residue;

(d) affects a part of the molecule distal to the binding site;

(e) is a substitution of one amino acid for another of similar size, charge, and/or hydrophobicity, and does not destroy a disulfide bond or other crosslink; and/or

(f) is at a site which is subject to substantial variation among a family of homologous proteins to which the protein of interest belongs.

These considerations can be used to design functional mutants.
Surface vs. Interior Residues

Charged amino acid residues almost always lie on the surface of the protein. For uncharged residues, there is less certainty, but in general, hydrophilic residues are partitioned to the surface and hydrophobic residues to the interior. Of course, for a membrane protein, the membrane-spanning segments are likely to be rich in hydrophobic residues.

Surface residues may be identified experimentally by various labeling techniques, or by 3-D structure mapping techniques like X-ray diffraction and NMR. A 3-D model of a homologous protein can be helpful.

### Binding Site Residues

Residues forming the binding site may be identified by (1) comparing the effects of labeling the surface residues before and after complexing the protein to its target, (2) labeling the binding site directly with affinity ligands, (3) fragmenting the protein and testing the fragments for binding activity, and (4) systematic mutagenesis (e.g., alanine-scanning mutagenesis) to determine which mutants destroy binding. If the binding site of a homologous protein is known, the binding site may be postulated by analogy.

Protein libraries may be constructed and screened that a large family (e.g., 10⁸) of related mutants may be evaluated simultaneously.

Hence, the mutations are preferably conservative modifications as defined below.

### “Substantially Identical”

A mutant protein (peptide) is substantially identical to a reference protein (peptide) if (a) it has at least 10% of a specific binding activity or a non-nutritional biological activity of the reference protein, and (b) is at least 50% identical in amino acid sequence to the reference protein (peptide). It is “substantially structurally identical” if condition (b) applies, regardless of (a).

Percentage amino acid identity is determined by aligning the mutant and reference sequences according to a rigorous dynamic programming algorithm which globally aligns their sequences to maximize their similarity, the similarity being scored as the sum of scores for each aligned pair according to an unbiased PAM250 matrix, and a penalty for each internal gap of −12 for the first null of the gap and 4 for each additional null of the same gap. The percentage identity is the number of matches expressed as a percentage of the adjusted (i.e., counting inserted nulls) length of the reference sequence.

More preferably, the sequence is not merely substantially identical but rather is at least 51%, at least 66%, at least 75%, at least 80%, at least 85%, at least 90%, at least 95%, at least 96%, at least 97%, at least 98% or at least 99% identical in sequence to the reference sequence.

### “Conservative Modifications”

“Conservative modifications” are defined as
(a) conservative substitutions of amino acids as hereafter defined; or
(b) single or multiple insertions (extension) or deletions (truncation) of amino acids at the termini.

Conservative modifications are preferred to other modifications. Conservative substitutions are preferred to other conservative modifications.

“Semi-Conservative Modifications” are modifications which are not conservative, but which are (a) semiconservative substitutions as hereafter defined; or (b) single or multiple insertions or deletions internally, but at interdomain boundaries, in loops or in other segments of relatively high mobility. Semi-conservative modifications are preferred to nonconservative modifications. Semi-conservative substitutions are preferred to other semi-conservative modifications.

Non-conservative substitutions are preferred to other non-conservative modifications.

The term “conservative” is used here in an a priori sense, i.e., modifications which would be expected to preserve 3D structure and activity, based on analysis of the naturally occurring families of homologous proteins and of past experience with the effects of deliberate mutagenesis, rather than post facto, a modification already known to conserve activity. Of course, a modification which is conservative a priori may, and usually is, also conservative post facto.

Preferably, except at the termini, no more than about five amino acids are inserted or deleted at a particular locus, and the modifications are outside regions known to contain binding sites important to activity. Preferably, insertions or deletions are limited to the termini.

A conservative substitution is a substitution of one amino acid for another of the same exchange group, the exchange groups being defined as follows

I Gly, Pro, Ser, Ala (Cys) (and any nonbiogenic, neutral amino acid with a hydrophobicity not exceeding that of the aforementioned a.a.'s)

II Arg, Lys, His (and any nonbiogenic, positively-charged amino acids)

III Asp, Glu, Asn, Gln (and any non biogenic negatively-charged amino acids)

IV Leu, Ile, Met, Val (Cys) (and any nonbiogenic, aliphatic, neutral amino acid with a hydrophobicity too high for I above)

V Phe, Trp, Tyr (and any nonbiogenic, aromatic neutral amino acid with a hydrophobicity too high for I above).

Note that Cys belongs to both I and IV.

Residues Pro, Gly and Cys have special conformational roles. Cys participates in formation of disulfide bonds. Gly imparts flexibility to the chain. Pro imparts rigidity to the chain and disrupts α helices. These residues may be essential in certain regions of the polypeptide, but substitutable elsewhere.

One, two or three conservative substitutions are more likely to be tolerated than a larger number.

“Semi-conservative substitutions” are defined herein as being substitutions within supergroup I/II/III or within supergroup IV/V, but not within a single one of groups I-V. They also include replacement of any other amino acid with alanine. If a substitution is not conservative, it preferably is semi-conservative.

“Non-conservative substitutions” are substitutions which are not “conservative” or “semi-conservative”.

“Highly conservative substitutions” are a subset of conservative substitutions, and are exchanges of amino acids within the groups Phe/Tyr/Trp, Met/Leu/Ile/Val, His/Arg/Lys, Asp/Glu and Ser/Thr/Ala. They are more likely to be tolerated than other conservative substitutions. Again, the smaller the number of substitutions, the more likely they are to be tolerated.

### “Conservatively Identical”

A protein (peptide) is conservatively identical to a reference protein (peptide) it differs from the latter, if at all, solely by conservative modifications, the protein (peptide remaining at least seven amino acids long if the reference protein (peptide) was at least seven amino acids long.

A protein is at least semi-conservatively identical to a reference protein (peptide) if it differs from the latter, if at all, solely by semi-conservative or conservative modifications.

A protein (peptide) is nearly conservatively identical to a reference protein (peptide) if it differs from the latter, if at all, solely by one or more conservative modifications and/or a single nonconservative substitution.

It is highly conservatively identical if it differs, if at all, solely by highly conservative substitutions. Highly conservatively identical proteins are preferred to those merely conservatively identical. An absolutely identical protein is even more preferred.

The core sequence of a reference protein (peptide) is the largest single fragment which retains at least 10% of a particular specific binding activity, if one is specified, or otherwise of at least one specific binding activity of the referent. If the referent has more than one specific binding activity, it may have more than one core sequence, and these may overlap or not.

If it is taught that a peptide of the present invention may have a particular similarity relationship (e.g., markedly identical) to a reference protein (peptide), preferred peptides are those which comprise a sequence having that relationship to a core sequence of the reference protein (peptide), but with internal insertions or deletions in either sequence excluded. Even more preferred peptides are those whose entire sequence has that relationship, with the same exclusion, to a core sequence of that reference protein (peptide).

### Nucleic Acid Sequences

A mutant DNA sequence is substantially identical to a reference DNA sequence if they are structural sequences, and encoding mutant and reference proteins which are substantially identical as described above.

If instead they are regulatory sequences, they are substantially identical if the mutant sequence has at least 10% of the regulatory activity of the reference sequence, and is at least 50% identical in nucleotide sequence to the reference sequence. Percentage identity is determined as for proteins except that matches are scored +5, mismatches −4, the gap open penalty is −12, and the gap extension penalty (per additional null) is −4.

DNA sequences may also be considered “substantially identical” if they hybridize to each other under stringent conditions, i.e., conditions at which the Tm of the heteroduplex of the one strand of the mutant DNA and the more complementary strand of the reference DNA is not in excess of 10° C. less than the Tm of the reference DNA homoduplex. Typically this will correspond to a percentage identity of 85-90%.

### Utility of Corresponding Nucleic Acid Sequences and Related Molecules

A DNA which encodes a favorable protein (or a functional mutant thereof) may be used in the production of that protein in vitro or in vivo (gene therapy). A DNA which encodes an unfavorable protein may be used in the production of that protein in vitro, and hence to facilitate the use of that protein as a diagnostic agent or as a target in screening for binding and neutralizing substances (antagonists).

If we wish to simply produce a favorable (or unfavorable) protein recombinantly, we can use any coding sequence, but preferably one with coding preferences matching those of the intended host. For gene therapy, if the gene endogenously encoding a favorable human protein of interest is not known, we can teach using any sequence encoding the human protein, but preferably one with human coding preferences. See, e.g., Desai, et al., “Intragenic codon bias in a set of mouse and human genes, Biol., 230(2): 215-25 (Sep. 21, 2004).

The DNAs of interest also include DNA sequences which encode peptide (including antibody) antagonists of the proteins of Master Table 1, subtables 1B or 1C.

A nucleic acid which specifically hybridizes to the human mRNA encoding a favorable human protein may be labeled or immobilized, and then used as a diagnostic agent in assays for that mRNA (or the corresponding cDNA). A nucleic acid which specifically hybridizes to the human mRNA encoding an unfavorable human protein may be used in a like manner, or it may be used therapeutically to inhibit the expression of that human protein.

For therapy, we have to know a part of the endogenous human gene encoding the unfavorable human protein (not necessarily the coding sequence). If it isn't, we can isolate the human gene using a probe designed on the basis of the known protein sequence. This could be a mixed probe, a probe with inosine in the degenerate positions, a guessed probe based on human codon preferences, or a combination of the above.

One form of therapy is anti-sense therapy. In this case, a single stranded nucleic acid molecule, which is complementary to the sense strand of the target sequence, is used as a therapeutic agent. The nucleic acid molecule may be DNA, RNA, or an analogue which is resistant to degradation.

Another form of therapy is RNAi therapy. This uses a double-stranded RNA molecule. Long (>200 nt) double stranded RNAs are known to silence the expression of target genes by their participation in the RNA interference (RNAi) pathway. The dsRNAs are processed into 20-25 nt small interfering RNAs (siRNAs) by the Dicer enzyme. The siRNAs assemble into RNA-induced silencing complexes (RISCs), and unwind. The siRNAs guide the RISCs to complementary messenger RNAs, which are subsequently degraded.

For therapeutic use, siRNAs can be prepared by direct chemical synthesis of the two strands, by in vitro transcription, or, in situ and in vivo, by siRNA expression vectors. The in vitro siRNAs may be delivered by any suitable means, including lipid-mediated transfection and electroporation.

A variety of algorithms have been developed for designing siRNAs (in particular, choosing their target sequence), see, e.g., Tuschi, “Expanding small RNA interference, Nature Biotechnol. 20:446-448 (2002). Preferred target sequences begin with AA dinucleotide and are 21 nt in length. More preferably, the target sequences have a 30-50% GC content, and are of high specificity to the target gene (e.g., not more than 16-17 contiguous pairs of homology to other genes). If the siRNA will be expressed from the RNA pol III promoter, it is preferable that the target sequence not contain stretches of four successive T's or four successive A's.

Once the target sequence is selected, the siRNA can be designed. Preferably, it comprises a hairpin structure, i.e., two inverted repeats (one binds the target sequence) which together form the stem of the hairpin structure, and a loop. The loop size is preferably 3-23 nt, and the published loop sequences include AUG, CCC<UUCG, CCACC, CTCGAG, AAGCUU, CCACACC, and UUCAAGAGA. The hairpin may optionally have a 5′ overhang.

One may also purchase siRNAs designed according to proprietary and supposedly more accurate algorithms from Ambion.

If the database DNA appears to be a full-length cDNA or gDNA, that is, it encodes an entire, functional, naturally occurring protein, then it may be used in the expression of that protein. Likewise, if the corresponding human gene is known in full-length, it may be used to express the human protein. Such expression may be in cell culture, with the protein subsequently isolated and administered exogenously to subjects who would benefit therefrom, or in vivo, i.e., administration by gene therapy. Naturally, any DNA encoding the same protein may be used for the same purpose, and a DNA encoding a protein which a fragment or a mutant of that naturally occurring protein which retains the desired activity, may be used for the purpose of producing the active fragment or mutant. The encoded protein of course has utility therapeutically and, in labeled or immobilized form, diagnostically.

If the database DNA appears to be a partial sequence (that is, partial relative to the DNA encoding the whole naturally occurring protein), then the database DNA may be used as a hybridization probe to isolate the full-length DNA from a suitable DNA (cDNA or gDNA) library. Stringent hybridization conditions are appropriate, that is, conditions in which the hybridization temperature is 5-10 deg. C. below the Tm of the DNA as a perfect duplex.

If the partial DNA encodes a biologically functional fragment of the cognate protein, it may be used in a manner similar to the full length DNA, i.e., to produce the functional fragment.

### Identification and Isolation of Homologous Genes Using a DNA Probe

If there is no human protein in the database which has a high BlastP score for alignment with the known mouse protein, then it is possible that the true human protein cognate has not yet been identified.

In this situation, if the mouse gene which encodes that mouse protein is known (which is almost always going to be the case), then the mouse gene (or its cDNA, or a fragment of the gene or cDNA) may be used experimentally to isolate the homologous human gene, and the human protein then deduced from the human gene. For particulars, see “genomics cases”.

### Identification of Binding Molecules, Especially Antagonists

Molecules which bind favorable and unfavorable proteins, or the corresponding nucleic acids, may be identified by screening libraries, especially combinatorial libraries, as described below. If the binding target is an unfavorable protein, or the corresponding nucleic acid, the binding molecules may further be screened for antagonist activity. The antagonism may be, e.g., at the receptor level or at the gene expression level. Combinatorial libraries of special interest are protein/peptide libraries (including antibody, antibody fragment and single chain antibody libraries), nucleic acid libraries, peptoid libraries, peptoid nucleic acid (PNA) libraries, and small organic molecule libraries.

### Library

The term “library” generally refers to a collection of chemical or biological entities which are related in origin, structure, and/or function, and which can be screened simultaneously for a property of interest. Libraries may be classified by how they are constructed (natural vs. artificial diversity; combinatorial vs. noncombinatorial), how they are screened (hybridization, expression, display), or by the nature of the screened library members (peptides, nucleic acids, etc.). For definitions of different types of libraries, see “genomics cases”.

### Combinatorial Libraries

The term “combinatorial library” refers to a library in which the individual members are either systematic or random combinations of a limited set of basic elements, the properties of each member being dependent on the choice and location of the elements incorporated into it. Typically, the members of the library are at least capable of being screened simultaneously. Randomization may be complete or partial; some positions may be randomized and others predetermined, and at random positions, the choices may be limited in a predetermined manner. The members of a combinatorial library may be oligomers or polymers of some kind, in which the variation occurs through the choice of monomeric building block at one or more positions of the oligomer or polymer, and possibly in terms of the connecting linkage, or the length of the oligomer or polymer, too.

Or the members may be nonoligomeric molecules with a standard core structure, like the 1,4-benzodiazepine structure, with the variation being introduced by the choice of substituents at particular variable sites on the core structure. Or the members may be nonoligomeric molecules assembled like a jigsaw puzzle, but wherein each piece has both one or more variable moieties (contributing to library diversity) and one or more constant moieties (providing the functionalities for coupling the piece in question to other pieces).

Thus, in a typical combinatorial library, chemical building blocks are at least partially randomly combined into a large number (as high as 10⁵) of different compounds, which are then simultaneously screened for binding (or other) activity against one or more targets.

In a “simple combinatorial library”, all of the members belong to the same class of compounds (e.g., peptides) and can be synthesized simultaneously. A “composite combinatorial library” is a mixture of two or more simple libraries, e.g., DNAs and peptides, or peptides, peptoids, and PNAs, or benzodiazepines and carbamates. The number of component simple libraries in a composite library will, of course, normally be smaller than the average number of members in each simple library, as otherwise the advantage of a library over individual synthesis is small. Libraries may be characterized by such parameters as size and diversity, see “genomics cases”. The library members may be presented as solutes in solution, or immobilized on some form of support. In the latter case, the support may be living (cell, virus) or nonliving (bead, plate, etc.). The supports may be separable (cells, virus particles, beads) so that binding and nonbinding members can be separated, or nonseparable (plate). In the latter case, the members will normally be placed on addressable positions on the support. The advantage of a soluble library is that there is no carrier moiety that could interfere with the binding of the members to the support. The advantage of an immobilized library is that it is easier to identify the structure of the members which were positive. When screening a soluble library, or one with a separable support, the target is usually immobilized. When screening a library on a nonseparable support, the target will usually be labeled.

Libraries of peptides (Smith, 1985), proteins (Ladner, U.S. Pat. No. 4,664,989), peptoids (Simon et al., Proc Natl Acad Sci USA, 89:9367-71 (1992)), nucleic acids (Ellington and Szostak, Nature, 246:818 (1990)), carbohydrates, and small organic molecules (Eichler et al., Med Res Rev, 15:481-96 (1995)) have been prepared or suggested for drug screening purposes.

There has been much interest in combinatorial libraries based on small molecules, which are more suited to pharmaceutical use, especially those which, like benzodiazepines, belong to a chemical class which has already yielded useful pharmacological agents. The techniques of combinatorial chemistry have been recognized as the most efficient means for finding small molecules that act on these targets. At present, small molecule combinatorial chemistry involves the synthesis of either pooled or discrete molecules that present varying arrays of functionality on a common scaffold. These compounds are grouped in libraries that are then screened against the target of interest either for binding or for inhibition of biological activity.

### Oligonucleotide Library

The library may be an library of oligonucleotides (linear, cyclic or branched), and these may include nucleotides modified to increase nuclease resistance and/or chemical stability. Libraries of potential anti-sense or RNAi molecules are of particular interest, but oligonucleotides can also be receptor antagonists.

### Peptide Library

The library may be a library of peptides, linear, cyclic or branched, and may or may not be limited in composition to the 20 genetically encoded amino acids. A peptide library may be an oligopeptide library or a protein library. Preferably, the oligopeptides are at least five, six, seven or eight amino acids in length. Preferably, they are composed of less than 50, more preferably less than 20 amino acids. In the case of an oligopeptide library, all or just some of the residues may be variable. The oligopeptide may be unconstrained, or constrained to a particular conformation by, e.g., the participation of constant cysteine residues in the formation of a constraining disulfide bond.

Proteins, like oligopeptides, are composed of a plurality of amino acids, but the term protein is usually reserved for longer peptides, which are able to fold into a stable conformation. A protein may be composed of two or more polypeptide chains, held together by covalent or noncovalent crosslinks. These may occur in a homooligomeric or a heterooligomeric state. A peptide is considered a protein if it (1) is at least 50 amino acids long, or (2) has at least two stabilizing covalent crosslinks (e.g., disulfide bonds). Thus, conotoxins are considered proteins.

Usually, the proteins of a protein library will be characterizable as having both constant residues (the same for all proteins in the library) and variable residues (which vary from member to member). This is simply because, for a given range of variation at each position, the sequence space (simple diversity) grows exponentially with the number of residue positions, so at some point it becomes inconvenient for all residues of a peptide to be variable positions. Since proteins are usually larger than oligopeptides, it is more common for protein libraries than oligopeptide libraries to feature variable positions. In the case of a protein library, it is desirable to focus the mutations at those sites which are tolerant of mutation. These may be determined by alanine scanning mutagenesis or by comparison of the protein sequence to that of homologous proteins of similar activity. It is also more likely that mutation of surface residues will directly affect binding. Surface residues may be determined by inspecting a 3D structure of the protein, or by labeling the surface and then ascertaining which residues have received labels. They may also be inferred by identifying regions of high hydrophilicity within the protein.

Because proteins are often altered at some sites but not others, protein libraries can be considered a special case of the biased peptide library.

There are several reasons that one might screen a protein library instead of an oligopeptide library, including (1) a particular protein, mutated in the library, has the desired activity to some degree already, and (2) the oligopeptides are not expected to have a sufficiently high affinity or specificity since they do not have a stable conformation. When the protein library is based on a parental protein which does not have the desired activity, the parental protein will usually be one which is of high stability (melting point >=50 deg. C.) and/or possessed of hypervariable regions.

Antibody libraries are of particular interest. The variable domains of an antibody possess hypervariable regions and hence, in some embodiments, the protein library comprises members which comprise a mutant of VH or VL chain, or a mutant of an antigen-specific binding fragment of such a chain. VH and VL chains are usually each about 110 amino acid residues, and are held in proximity by a disulfide bond between the adjoing CL and CH1 regions to form a variable domain. Together, the VH, VL, CL and CH1 form an Fab fragment. In human heavy chains, the hypervariable regions are at 31-35, 49-65, 98-111 and 84-88, but only the first three are involved in antigen binding. There is variation among VH and VL chains at residues outside the hypervariable regions, but to a much lesser degree. A sequence is considered a mutant of a VH or VL chain if it is at least 80% identical to a naturally occurring VH or VL chain at all residues outside the hypervariable region. In a preferred embodiment, such antibody library members comprise both at least one VH chain and at least one VL chain, at least one of which is a mutant chain, and which chains may be derived from the same or different antibodies. The VH and VL chains may be covalently joined by a suitable linker moiety, as in a “single chain antibody”, or they may be noncovalently joined, as in a naturally occurring variable domain. If the joining is noncovalent, and the library is displayed on cells or virus, then either the VH or the VL chain may be fused to the carrier surface/coat protein. The complementary chain may be co-expressed, or added exogenously to the library. The members may further comprise some or all of an antibody constant heavy and/or constant light chain, or a mutant thereof.

### Peptoid Library

A peptoid is an analogue of a peptide in which one or more of the peptide bonds (—NH—O—) are replaced by pseudopeptide bonds, which may be the same or different. It is not necessary that all of the peptide bonds be replaced, i.e., a peptoid may include one or more conventional amino acid residues, e.g., proline.

A peptide bond has two small divalent linker elements, —NH— and —CO—. Thus, a preferred class of psuedopeptide bonds are those which consist of two small divalent linker elements. Each may be chosen independently from the group consisting of amine (—NH—), substituted amine (—NR—), carbonyl (—CO—), thiocarbonyl (—CS—), methylene (—CH2-), monosubstituted methylene (—CHR—), disubstituted methylene (—CR1R2-), ether (—O—) and thioether (—S—). The more preferred pseudopeptide bonds include:
N-modified —NRCO—
Carba Ψ —CH₂—CH₂—
Depsi Ψ —CO—O—
Hydroxyethylene Ψ —CHOH—CH₂—
Ketomethylene Ψ —CO—CH₂—
Methylene-Oxy —CH₂—O—
Reduced —CH₂—NH—
Thiomethylene —CH₂—S—
Thiopeptide —CS—NH—
Retro-Inverso —CO—NH—

A single peptoid molecule may include more than one kind of pseudopeptide bond. For the purposes of introducing diversity into a peptoid library, one may vary (1) the side chains attached to the core main chain atoms of the monomers linked by the pseudopeptide bonds, and/or (2) the side chains (e.g., the —R of an —NRCO—) of the pseudopeptide bonds. Thus, in one embodiment, the monomeric units which are not amino acid residues are of the structure —NR1-CR2-CO—, where at least one of R1 and R2 are not hydrogen. If there is variability in the pseudopeptide bond, this is most conveniently done by using an —NRCO— or other pseudopeptide bond with an R group, and varying the R group. In this event, the R group will usually be any of the side chains characterizing the amino acids of peptides, as previously discussed.

If the R group of the pseudopeptide bond is not variable, it will usually be small, e.g., not more than 10 atoms (e.g., hydroxyl, amino, carboxyl, methyl, ethyl, propyl). If the conjugation chemistries are compatible, a simple combinatorial library may include both peptides and peptoids.

### Peptide Nucleic Acid Library

PNA oligomer libraries have been made; see e.g. Cook, 6,204,326. A PNA oligomer is here defined as one comprising a plurality of units, at least one of which is a PNA monomer which comprises a side chain comprising a nucleobase. For nucleobases, see U.S. Pat. No. 6,077,835. The classic PNA oligomer is composed of (2-aminoethyl)glycine units, with nucleobases attached by methylene carbonyl linkers. That is, it has the structure

H—(—HN—CH₂—CH₂—N(—CO—CH₂—B)—CH₂—CO—)ₐ—OH

where the outer parenthesized substructure is the PNA monomer.

In this structure, the nucleobase B is separated from the backbone N by three bonds, and the points of attachment of the side chains are separated by six bonds. The nucleobase may be any of the bases included in the nucleotides discussed in connection with oligonucleotide libraries. The bases of nucleotides A, G, T, C and U are preferred. A PNA oligomer may further comprise one or more amino acid residues, especially glycine and proline.

One can readily envision related molecules in which (1) the —COCH2- linker is replaced by another linker, especially one composed of two small divalent linkers as defined previously, (2) a side chain is attached to one of the three main chain carbons not participating in the peptide bond (either instead or in addition to the side chain attached to the N of the classic PNA); and/or (3) the peptide bonds are replaced by pseudopeptide bonds as disclosed previously in the context of peptoids.

### Small Organic Compound Library

The small organic compound library (“compound library”, for short) is a combinatorial library whose members are suitable for use as drugs if, indeed, they have the ability to mediate a biological activity of the target protein. Bunin, et al. generated a 1,4-benzodiazepine library of 11,200 different 2-aminobenzophenone derivatives prepared from 20 acid chlorides, 35 amino acids, and 16 alkylating agents. See Bunin, et al., Proc. Nat. Acad. Sci. USA, 91:4708 (1994). Since only a few 2-aminobenzophenone derivatives are commercially available, it was later disjoined into 2-aminoarylstannane, an acid chloride, an amino acid, and an alkylating agent. Bunin, et al., Meth. Enzymol., 267:448 (1996). The arylstannane may be considered the core structure upon which the other moieties are substituted, or all four may be considered equals which are conjoined to make each library member.

Heterocylic combinatorial libraries are reviewed generally in Nefzi, et al., Chem. Rev., 97:449-472 (1997). Examples of candidate simple libraries which might be evaluated include derivatives of the following:

Cyclic Compounds Containing One Hetero Atom
Heteronitrogen
   pyrroles
      pentasubstituted pyrroles
   pyrrolidines
   pyrrolines
   prolines
   indoles
   beta-carbolines'
   pyridines
      dihydropyridines
      1,4-dihydropyridines
      pyrido[2,3-d]pyrimidines
      tetrahydro-3H-imidazo[4,5-c]pyridines
   Isoquinolines
      tetrahydroisoquinolines
   quinolones
   beta-lactams
      azabicyclo[4.3.0]nonen-8-one amino acid
Heterooxygen
   furans
      tetrahydrofurans
         2,5-disubstituted tetrahydrofurans pyrans
      hydroxypyranones
      tetrahydroxypyranones
   gamma-butyrolactones
Heterosulfur
   sulfolenes

Cyclic Compounds with Two or More Hetero atoms
Multiple heteronitrogens
   imidazoles
   pyrazoles
   piperazines
      diketopiperazines
      arylpiperazines
      benzylpiperazines
   benzodiazepines
   1,4-benzodiazepine-2,5-diones
   hydantoins
      5-alkoxyhydantoins
   dihydropyrimidines
   1,3-disubstituted-5,6-dihydropyrimidine-2,4-diones
   cyclic ureas
   cyclic thioureas
   quinazolines
      chiral 3-substituted-quinazoline-2,4-diones triazoles
      1,2,3-triazoles
   purines
Heteronitrogen and Heterooxygen
   dikelomorpholines
   isoxazoles
   isoxazolines
Heteronitrogen and Heterosulfur
   thiazolidines
      N-axylthiazolidines
   dihydrothiazoles
      2-methylene-2,3-dihydrothiazates
      2-aminothiazoles
   thiophenes
      3-amino thiophenes
   4-thiazolidinones
   4-melathiazanones
   benzisothiazolones

For details on synthesis of libraries, see Nefzi, et al., Chem. Rev., 97:449-72 (1997), and references cited therein.

For further information on small organic compound combinatorial libraries, see “genomics cases”.

### Pharmaceutical Methods and Preparations

The preferred animal subject of the present invention is a mammal. By the term “mammal” is meant an individual belonging to the class Mammalia. The invention is particularly useful in the treatment of human subjects, although it is intended for veterinary uses as well. Preferred nonhuman subjects are of the orders Primata (e.g., apes and monkeys), Artiodactyla or Perissodactyla (e.g., cows, pigs, sheep, horses, goats), Carnivora (e.g., cats, dogs), Rodenta (e.g., rats, mice, guinea pigs, hamsters), Lagomorpha (e.g., rabbits) or other pet, farm or laboratory mammals.

The term “protection” as used herein, is intended to include “prevention,” “suppression” and “treatment.” Unless qualified, the term “prevention” will be understood to refer to both prevention of the induction of the disease, and to suppression of the disease before it manifests itself clinically. The preventative or prophylactic use of a pharmaceutical usually involves identifying subjects who are at higher risk than the general population of contracting the disease, and administering the pharmaceutical to them in advance of the clinical appearance of the disease. The effectiveness of such use is measured by comparing the subsequent incidence or severity of the disease, or of particular symptoms of the disease, in the treated subjects against that in untreated subjects of the same high risk group.

While high risk factors vary from disease to disease, in general, these include (1) prior occurrence of the disease in one or more members of the same family, or, in the case of a contagious disease, in individuals with whom the subject has come into potentially contagious contact at a time when the earlier victim was likely to be contagious, (2) a prior occurrence of the disease in the subject, (3) prior occurrence of a related disease, or a condition known to increase the likelihood of the disease, in the subject; (4) appearance of a suspicious level of a marker of the disease, or a related disease or condition; (5) a subject who is immunologically compromised, e.g., by radiation treatment, HIV infection, drug use, etc., or (6) membership in a particular group (e.g., a particular age, sex, race, ethnic group, etc.) which has been epidemiologically associated with that disease.

In some cases, it may be desirable to provide prophylaxis for the general population, and not just a high risk group. This is most likely to be the case when essentially all are at risk of contracting the disease, the effects of the disease are serious, the therapeutic index of the prophylactic agent is high, and the cost of the agent is low.

A prophylaxis or treatment may be curative, that is, directed at the underlying cause of a disease, or ameliorative, that is, directed at the symptoms of the disease, especially those which reduce the quality of life.

It should also be understood that to be useful, the protection provided need not be absolute, provided that it is sufficient to carry clinical value. An agent which provides protection to a lesser degree than do competitive agents may still be of value if the other agents are ineffective for a particular individual, if it can be used in combination with other agents to enhance the level of protection, or if it is safer than competitive agents. It is desirable that there be a statistically significant (p=0.05 or less) improvement in the treated subject relative to an appropriate untreated control, and it is desirable that this improvement be at least 10%, more preferably at least 25%, still more preferably at least 50%, even more preferably at least 100%, in some indicia of the incidence or severity of the disease or of at least one symptom of the disease.

At least one of the drugs of the present invention may be administered, by any means that achieve their intended purpose, to protect a subject against a disease or other adverse condition. The form of administration may be systemic or topical. For example, administration of such a composition may be by various parenteral routes such as subcutaneous, intravenous, intradermal, intramuscular, intraperitoneal, intranasal, transdermal, or buccal routes. Alternatively, or concurrently, administration may be by the oral route. Parenteral administration can be by bolus injection or by gradual perfusion over time.

A typical regimen comprises administration of an effective amount of the drug, administered over a period ranging from a single dose, to dosing over a period of hours, days, weeks, months, or years.

It is understood that the suitable dosage of a drug of the present invention will be dependent upon the age, sex, health, and weight of the recipient, kind of concurrent treatment, if any, frequency of treatment, and the nature of the effect desired. However, the most preferred dosage can be tailored to the individual subject, as is understood and determinable by one of skill in the art, without undue experimentation. This will typically involve adjustment of a standard dose, e.g., reduction of the dose if the patient has a low body weight.

Prior to use in humans, a drug will first be evaluated for safety and efficacy in laboratory animals. In human clinical studies, one would begin with a dose expected to be safe in humans, based on the preclinical data for the drug in question, and on customary doses for analogous drugs (if any). If this dose is effective, the dosage may be decreased, to determine the minimum effective dose, if desired. If this dose is ineffective, it will be cautiously increased, with the patients monitored for signs of side effects.

The total dose required for each treatment may be administered by multiple doses or in a single dose. The protein may be administered alone or in conjunction with other therapeutics directed to the disease or directed to other symptoms thereof. Typical pharmaceutical doses, for adult humans, are in the range of 1 ng to 10 g per day, more often 1 mg to 1 g per day. The appropriate dosage form will depend on the disease, the pharmaceutical, and the mode of administration; possibilities include tablets, capsules, lozenges, dental pastes, suppositories, inhalants, solutions, ointments and parenteral depots.

In the case of peptide drugs, the drug may be administered in the form of an expression vector comprising a nucleic acid encoding the peptide; such a vector, after incorporation into the genetic complement of a cell of the patient, directs synthesis of the peptide. Suitable vectors include genetically engineered poxviruses (vaccinia), adenoviruses, adeno-associated viruses, herpesviruses and lentiviruses which are or have been rendered nonpathogenic.

### Assay Compositions and Methods

The compounds of the present invention may be used, in labeled or immobilized form, as assay reagents. For assay formats, signal producing systems, labels and supports, please see “genomics cases”, hereby incorporated by reference in their entirety.

### Example 1

We are utilizing a mouse model of diet-induced obesity that progresses to diabetes. The diet is high in fat, an increasing component in the U.S. diet, and has been documented to lead to diabetes in C57BL/6J mice (Surwit et al., 1988). After weaning, C57BL/6J mice were fed either the high fat (HF) diet or a standard lab chow diet. Body weight was monitored bi-weekly. Fasting glucose and insulin levels were measured after various periods of time after commencement of the high fat diet. Consumption of the HF diet resulted in significant, progressive increases in body weight and fasting insulin levels in comparison to consumption of the Std diet. Fasting glucose levels of mice on the HF diet were dramatically increased at the first time point assayed (2 weeks) and remained high through the duration of the experiment. At each time point, several diabetic and control mice were sacrificed and a number of tissues collected.

### Overview

Male mice were reared on a normal or high-fat custom purified diet to ensure reproducibility and comparable nutrition. Tissues were harvested at regular intervals during the onset and progression of obesity and type 2 diabetes. Each tissue sample were divided for concurrent histology and proteomic studies. In the proteomics studies, separation and visualization of the proteins at a specific time in a specific tissue or “tissue specific protein profile” were established by two-dimensional gel electrophoresis and the relative abundance of each protein were determined by densitometry. Proteins that are differentially expressed or modified as a consequence of obesity and diabetes were excised from the gels and analyzed by mass spectrometry. (Predictions based on the peptide mass fingerprints and deductive reasoning were can be confirmed by western blot analysis and/or immunohistochemistry.)

### Experimental Animals

Obesity and subsequent hyperinsulinemia and hyperglycemia were induced by feeding a group of 3 week old mice (50 C57BL/6 males) a high-fat diet (Bio-Serve, Frenchtown, N.J., #F1850 High Carbohydrate-High Fat; 56% of calories from fat, 16% from protein and 27% from carbohydrates). Another group of 3 week old mice (20 C57BL/6 males) were fed the normal control diet PMI Nutrition International Inc., Brentwood, Mo., Prolab RMH3000; 14% of calories from fat, 16% from protein and 60% from carbohydrates). The mice were placed onto the respective diets immediately following weaning. Animal weights were determined weekly. Fasting blood-glucose and plasma insulin measurements were determined after 2, 4, and 8 weeks, and then every other month, on the respective diets. Two of the “most typical” animals were selected for each group (Control, hyperinsulinemic and Diabetic) at each time point for sacrifice. The selected mice were sacrificed and tissues obtained.

### Glucose Homeostasis and Blood Chemistry

Experimental animals, except those designated for advanced phenotypic analysis, were monitored for glucose homeostasis to assess the stage and severity of obesity and diabetes. Measurements were taken at 2, 4, and 8 weeks, and then every other month.

Fasting blood glucose levels were determined the day after weighing the animal.

Following 4 or 8 hours of food deprivation, fasting blood glucose levels were measured from a drop of blood using a OneTouch glucometer from Lifescan (Milpitas, Calif.). All measurements will occur between 2:00 and 4:00 PM. Similarly, intraperitoneal (IP) glucose tolerance tests (IPGTTs) were initiated immediately after a 4-hour fasting period by IP injection of 25% glucose solution administered at a volume of 0.01 ml/g body weight. Blood-glucose was measured at 30, 60, 90, and 120 minutes after the injection.

Blood is collected from the tail vein of fasted mice, between 2 p.m. and 4 p.m., using a heparinized capillary tube and stored on ice. Plasma was separated from the cellular components by centrifugation for 10 minutes at 7000×g and then stored at −80° C. Insulin concentrations were determined using the Ultra-Sensitive Rat Rnsulin ELISA kit and rat insulin standards (both from ALPCO: Windham, N.H.), essentially as instructed by the manufacturer. Values were adjusted by a factor of 1.23 (as determined by the manufacturer) to correct for species differences in the antibody.

Normal weight, normal fasting blood glucose and normal fasting plasma insulin levels are defined as the respective mean values of the animals fed the control diet.

### Classification of Animals

During the onset and progression of obesity and diabetes, the animals were classified according to these phenotypes: (1) normal, (2) obese, (3) obese/hyperinsulinemic (4) obese/hyperinsulinemic/diabetic., according to the definitions set forth prior to the Examples herein.

### Tissue Isolation and Preparation

The mice were sacrificed at the appropriate times and the 16 different tissues (Liver, Gastrocnemius, Pancreas, Epididymal Fat, Subcutaneous Fat, Kidney, Stomach Brain, Tongue, Heart, Skin, Small Intestine, Testes, Spleen, Bone & Serum) are harvested. All tissues were harvested at regular intervals for up to 14 time-points during the onset and progression of obesity and type 2 diabetes. Mice were sacrificed by cervical dislocation in the absence of anesthesia (Euthanasia will be by CO₂ inhalation for animals that are deemed to be suffering.)

Each organ is quickly removed and weighed and then maintained on ice during the dissection. This is desirable for the simultaneous preservation of multiple tissues for three distinct applications: proteomics, histology, and RNA analysis. The tissue was placed in 10% formalin for histology or frozen in cryogenic vials with liquid nitrogen for proteomics and RNA analysis.

Tissues were dissected in a manner which struck a balance between speed and specificity. The brain, for example, is divided into two hemisphere and each hemisphere is divided into cortex, cerebellum, and midbrain, but the liver is not separated into lobes and the heart is not separated into individual chambers.

Muscle, skin, WAT, and heart were homogenized in IEF buffer containing non-ionic chaotropes (7 M urea and 2 M thiourea) and zwitterionic detergent (2-4% CHAPS), whereas kidney, liver, pancreas, and brain were lysed by dounce homogenization with a tight-fitting pestle in ice cold sterile lysis buffer containing 0.25 M Sucrose, 50 mM Tris-HCl pH 7.6, 25 mM KCl, 5 mM MgCl₂, 2 mM DTT and protease inhibitor cocktail (94).

The homogenate was placed in tubes and centrifuged at 25,000 rpm (Beckman LE 30) to remove nuclei and other organelles. The supernatant was layered over a 1.5 ml cushion of lysis buffer containing 30% (w/v) sucrose and centrifuged in Beckman LE 80 at 36,000 rpm (130,000 g) for 2.5 hr at 4° C. using SW60 rotor. The supernatant (S130) was aliquoted, and stored at −80° C. After removal of the sucrose interface, the polysomal pellet was rinsed twice and then resuspended in ˜250 μl of lysis buffer. Samples were maintained on ice until aliquoted, frozen on dry ice and stored at −80° C. for subsequent use.

Serum was collected by decapitation following cervical dislocation. After removal of cellular component by centrifugation at 7000×g for 10 min, serum was stored at −80° C. For isoelectric focusing (IEF), serum was mixed with IEF buffer followed by reduction and Alkylation.

The protein concentration of each preparation was determined by spectrophotometry (Beckman DU-640) using the Bradford method (BioRad) or the Lowry method. Typically, these fractions yield 100-3000 μl samples containing 7-12 μg protein/μl. The yield for crude tissue homogenates ranges from 500 μl at a concentration of ˜22 μg/μl for white adipose tissue to about ˜50 μg/μl in 2 ml for liver and skeletal muscle. Serum protein samples were diluted with sample buffer (5 M urea, 2M thiourea, 2% CHAPS, 2% SB3-10, 0.1% Bio-lytes, 50 mM Tris/HCl pH 8.8) at final concentration of up to 4 mg/ml. Protein was reduced by tributyl phosphine (TBP) for 2 hours at room temperature to break disulfide bonds. Alkylating agent, iodoacetamide (IAA; 3.2 mg/ml), was added to prevent spontaneous re-oxidation of disulfide bonds.

The alkylated samples were added to immobilized pH gradient (IPG) strips (Bio-rad) and focused at 4000V for 20,000-30,000 V hrs. The second dimension separation was performed by SDS polyacrylamide gel electrophoresis (SDS-PAGE), which separates proteins based on their masses.

We achieve excellent resolution using 200-500 μg. for two-dimensional gel-electrophoresis experiments. The protein concentrations used may vary depending on the objectives of the experiment. For example, higher concentrations may be used at the expense of resolution in order to harvest enough protein for micro-sequencing or production of antibodies.

Separated proteins in the gels were fixed (40% ethanol, 2% acetic acid and 0.0005% SDS) and stained using SYPRO Orange (Molecular Probes, Inc., Eugene, Oreg.) fluorescent dye. See Lopez M F, Berggren K, Chemokalskaya E, Lazarev A, Robinson M, Patton W F “A comparison of silver stain and SYPRO Ruby Protein Gel Stain with respect to protein detection in two-dimensional gels and identification by peptide mass profiling”, Electrophoresis 21:3673-83 (2000); Malone J P, Radabaugh M R, Leimgruber R M, Gerstenecker GS, “Practical aspects of fluorescent staining for proteomic applications”, Electrophoresis 22:919-32 (2001).

### Spot Detection and Image Comparison

Gel images were captured with a high-resolution CCD camera (e.g. Versa-Doc 3000, Bio-Rad) or a laser-scanning device (Fuji FLA-3000G). PDQuest image analysis software package from Bio-Rad was used to interpret and quantify 2-D gel patterns. Before comparing spot quantities between gels, each gel image was optimized and adjusted for image background, spot intensity, streaking, etc., and then normalized to compensate for any variation in spot intensity that is not due to differential protein expression, i.e., variation caused by loading, staining, and imaging between gels. The spot detection wizard function of the PDQuest software helped to optimize the conditions needed to detect all the spots in the gel.

The end result of spot detection was three separate images of the same gel: the original gel scan, which is unchanged; the filtered image, with noise and background removed; and a synthetic image, containing ideal Gaussian representations of the spots in the original scan. These Gaussian spots were used for matching and quantization.

### Protein Identification

Using these images as a guide, proteins of interest were manually excised from the gels and prepared for analysis by mass spectrometry (107,108). Matrix-Assisted Laser Desorbtion/Ionization-Time of Flight (MALDI-TOF) is the method of choice because it is highly sensitive and compatible with protein sequencing reactions. Protein samples (individual gel spots) were digested with trypsin and subjected to mass spectrometric analysis by MALDI-TOF (Voyager-DE Pro, The Applied Biosystems). A peak list was extracted from each mass spectrum obtained by MALDI-TOF and submitted to Matrix. Science's Mascot (http://www.matrixscience.com) for a preliminary database search.

In addition to MALDI-TOF, up to six tryptic peptide peaks were selected and further analyzed by MS/MS (4700 Proteomics Analyzer, The Applied Biosystems).

The gel spot location data is formatted to facilitate comparisons between gels and with the proteomic databases such as those maintained by the Danish Centre for Human Genome Research at the University of Aarhus (105) (http://biobase.dk/cgi-bin/celis) and the ExPASy (Expert Protein Analysis System) proteomics server (http://www.exnasv.org/) maintained by the Swiss Institute of Bioinformatics (SIB) at the University of Geneva (106). These databases have tools designed to overcome the enormous computational challenges associated with proteomic analysis. For example, it is possible to search databases (e.g. SWISS-PROT, TrEMBL) for proteins whose theoretical isoelectric point (PI), molecular weight (Mw), amino acid composition or peptide mass fingerprint match experimentally derived data. Additional tools predict post-translational modifications and protein structure.

Our principal software resources were Matrix Science's Mascot (http://www.matrixscience.com) and the Protein Prospector suite of tools located at UCSF http://prospector.ucsf.edu), which have probability-based peptide mass fingerprint (PMF) database search tools and MS/MS search tools.

The quality of each mass spectrum was assessed in terms of resolution and noise. When the mass spectrum was of sufficient quality to use for an analysis, it was assessed for common additional peaks corresponding to peptides from the auto-digestion of trypsin or matrix molecules and keratins contamination. Once a spectrum of acceptable quality was obtained, a peak list was generated for database search.

### Software Analysis of Mass Spectra

The NCBI database was searched using the specified software. For database search, the following parameters were specified for MASCOT MS analysis:
1. Maximum of I missed cleavage by trypsin.
2. Cys Modified by Carbamidomethylation.
3. Possible Modifications of “Peptide N-terminal Gln to pyroGlu+Oxidation of M+Protein N-terminus Acetylated.” (Default setting)
4. Species choose of “All” to avoid missing improperly annotated data.
5. MW and pI range not specified.
6. Peptide masses are monoisotopic.
7. Contaminant Masses were list of all trypsin autolysis peaks present in the spectrum, as well as any keratin or other known contaminant peaks in the spectrum such as matrix material peaks.
8. Mass tolerance: instrument dependent, typically 50 ppm or better.

The appropriate parameters for an MS-Fit search are same as those listed above for the Mascot MS search. If an MS-Fit option is not listed above, then the default setting is appropriate.

When evaluating the search results, it is important to remember that the top hit is not necessarily a good hit, nor is it necessarily the correct hit. A number of factors need to be considered.

Species. If the search was performed on the entire database, then a hit could be on a non-mouse protein. Such a hit would ordinarily be disregarded, unless there was reason to think either that the database protein was improperly annotated, or that the gel protein was a contaminant.

Score. A score above ˜10̂5 is generally considered a good hit, but this is not always the case. For MS-Fit, there is no absolute value for a score that makes the hit a certainty. Furthermore, a low score does not necessarily indicate that the hit is not the correct hit; it simply indicates that the identification should not be used without-further confirmation. MS/MS analysis has confirmed identifications for PMF hits with scores as low as 50.

Mass errors. Mass errors should be somewhat uniform.

% Coverage—Typical % coverage for MALDI data is 20-50%. Higher % coverage indicates that a larger portion of the protein was accounted for by the peptides observed in the spectrum. Very low % coverage, in combination with a large protein MW was considered to be a spurious hit.

Location in the protein of the peptides that were matched. When peptides that were all located in one end of the protein was considered to be a truncated form of the protein and explained inconsistencies between the experimental and theoretical MW and pI values.

If a sufficiently high score was returned for protein identification and the majority of the peaks are accounted for by the hit, the analysis was stopped at this point. If a number of prominent peaks in the spectrum were unaccounted for by the first protein hit, a search on unmatched peaks was performed to search for a second component. If the score was not satisfactory, up to six peaks that were analyzed via MS/MS to confirm the identification. MS/MS is the selection of a single peptide from the tryptic digest by the mass spectrometer, followed by the fragmentation of that peptide within the mass spectrometer and the acquisition of its fragment ion spectrum. Given that the fragmentation pattern of a given peptide is specific to its sequence and that the mass of the intact peptide is known, identification obtained by only one or two peptides in the PMF spectrum is often considered accurate.

If no reasonable PMF hits were returned for an otherwise good spectrum, MS/MS analysis was used for protein identification or to confirm the MS analysis. In addition to identifying proteins via database searches, MS/MS can be used to provide sequence information that can be used for BLAST searching, or identify the presence or location of post-translational modifications.

### MS/MS Data Analysis

For searching on MS/MS data the primary tool used was the Mascot from Matrix Science. The search tool performs a theoretical (in silico) digestion of the proteins in the database using the selected enzyme, generating a list of theoretical peptides for each protein. When the MS/MS peak list is submitted to the database, the parent ion is compared to the results from the in silico digestion. A theoretical fragmentation is carried out on all peptides from the in silico digestion that are within the selected mass tolerance of the parent ion. The ion types that are calculated (alpha, beta, gamma, etc.) are determined by the parameters selected in the search. In Mascot, these ion types are determined by the selection of instrument type. In MS-Tag, the ion types may be individually selected, or they are determined by the instrument type selected. The peak list is then compared against the masses generated by the theoretical fragmentation to determine if the fragmentation pattern of a peptide in the database matches the spectrum. If a matching peptide is found, a ranking or score is generated.

The search parameters used for the MS/MS searches are

1) Database —NCBI is more complete, but SwissProt is more highly annotated and faster to search. NCBI has more entries for certain species, such as mouse and human.
2) Taxonomy—Select the desired species or choose “All.”
3) Enzyme—Select Trypsin unless a different enzyme was used in the analysis. If non-specific cleavage is suspected, choose No Enzyme.
4) Allow up to xx Missed Cleavages—Typically I missed cleavage will suffice.

### 5) Fixed and Variable Modifications:

a. When a search is performed by the MPC, modifications are not considered in the first pass. Although the protein hit will generally be the same when whether modifications are chosen or not (since the peptides usually are not modified), the score is generally higher for the same hit when modifications are not considered.
b. If no hit is obtained without considering modifications, a second search is performed and the following modifications are chosen under the “variable modifications”: Acetyl-N-term, Carbamidomethylation (assuming the proteins have been reduced and alkylated), Oxidation-M (methionine), and Pyroglu (N-term-Q). Other modifications should be selected as appropriate.
6) Peptide Tolerance—Parent ion mass accuracy should be 50 ppm or better.
7) MS/MS Tolerance—Fragment ion mass accuracy may differ for TOF/TOF and MALDI QTOF data, but is typically better than 0.5 Da. The MALDI QTOF is calibrated for acquiring PMF spectra, and so the calibration at the low end of the mass spectrum (below 500 Da) may have a higher mass error than the rest of the spectrum. The TOF/TOF has separate MS and MS/MS calibrations and should be relatively consistent across the entire mass range.
8) Peptide Charge—+1 for MALDI MS/MS data.
9) Data Format—The MALDI QTOF peak list files are in the Micromass format. TOF/TOF data will be in mass intensity pairs and must be converted to the Mascot Generic format, which is described on the Mascot website.
10) Instrument—Chose MALDI-QUAD-TOF or MALDI TOF-TOF as appropriate.

The results of the Mascot MS, Mascot MS/MS and MSFIT analyses of each spot are shown in Master Tables 101-103. Multiple hits on the same protein are a strong indication of a positive identification.

### Identification of Corresponding Human Proteins

Database Searches Nucleotide sequences and predicted amino acid sequences were compared to public domain databases using the Blast 2.0 program (National Center for Biotechnology Information, National Institutes of Health).

Protein database searches were conducted with the then-current version of BLAST P, see Altschul et al. (1997), supra. Searches employed the default parameters, unless otherwise stated. The scoring matrix was BLOSUM62, with gap costs of II for existence and 1 for extension. Results are shown in Master Table 1.

“ref” indicates that NCBI's RefSeq is the source database. The identifier that follows is a RefSeq accession number, not a GenBank accession number. “RefSeq sequences are derived from GenBank and provide non-redundant curated data representing our current knowledge of known genes. Some records include additional sequence information that was never submitted to an archival database but is available in the literature. A small number of sequences are provided through collaboration; the underlying primary sequence data is available in GenBank, but may not be available in any one GenBank record. RefSeq sequences are not submitted primary sequences. RefSeq records are owned by NCBI and therefore can be updated as needed to maintain current annotation or to incorporate additional sequence information.” See also http://www.ncbi.nlm.nih.gov/LocusLink/refseq.html

It will be appreciated by those in the art that the exact results of a database search will change from day to day, as new sequences are added. Also, if you query with a longer version of the original sequence, the results will change. The results given here were obtained at one time and no guarantee is made that the exact same hits would be obtained in a search on the filing date. However, if an alignment between a particular query sequence and a particular database sequence is discussed, that alignment should not change (if the parameters and sequences remain unchanged).

### Northern Analysis.

Northern analysis may be used to confirm the results. Favorable and unfavorable genes, identified as described above, or fragments thereof, will be used as probes in Northern hybridization analyses to confirm their differential expression. Total RNA isolated from subject mice will be resolved by agarose gel electrophoresis through a 1% agarose, 1% formaldehyde denaturing gel, transferred to positively charged nylon membrane, and hybridized to a probe labeled with [32P] dCTP that was generated from the aforementioned gene or fragment using the Random Primed DNA Labeling Kit (Roche, Palo Alto, Calif.), or to a probe labeled with digoxigenin (Roche Molecular Biochemicals, Indianapolis, Ind.), according to the manufacturer's instructions.

### Transgenic Animals.

Transgenic expression may be used to confirm the favorable or unfavorable role of the identified mouse or human protein. In one embodiment, a mouse is engineered to overexpress the favorable or unfavorable mouse protein in question. In another embodiment, a mouse is engineered to express the corresponding favorable or unfavorable human protein. In a third embodiment, a nonhuman animal other than a mouse, such as a rat, rabbit, goat, sheep or pig, is engineered to express the favorable or unfavorable mouse or human protein.

### Results

For the identification of particular spots as particular mouse proteins, see Master Tables 101-103. For the identification of the corresponding human proteins, see Master Table 1. Both tables set forth the differential expression values for the mouse proteins.

### Discussion

Diets: In humans, obesity-induced type 2 diabetes often involves a progression from a normal phenotype through an insulin resistant/hyperinsulinemic state to overt diabetes. These stages are replicated in C57BL/6J mice fed a diet composed of 58% kcal from fat (Bioserve F1850)(75). These mice become relatively obese, and often develop hyperinsulinemia (=0.67 ng/ml) and diabetes (fasting glucose=190 mg/dl). This regimen clearly induces a phenotype that resembles type 2 diabetes.

Tissue Collection: We do not attempt to isolate distinct cells such as neurons and glia in brain tissue or β-islet cells in pancreas, in part because our rapid dissection procedures are desirable to ensure the integrity of the tissue for multiple applications. We are confident that the loss of cell-type specificity will not interfere with our ability to detect important tissue-specific changes that are attributable to stage and severity of obesity and diabetes.

We also realize that all of our freshly collected solid tissue samples may be contaminated by minor amounts of blood and vasculature and that some of the putative tissue-specific differences in gene expression may be attributable to proteins derived from the vasculature used to perfuse the harvested tissue. Given the importance of the vascular endothelium to the development of diabetes-related complications, this may actually provide important information. In any event, our principal goal is the identification of favorable and unfavorable proteins as defined above, regardless of tissue specificity.

Experiments can be conducted to confirm the relationship between mRNA abundance and actual gene expression. When desirable, we will probe tissue slices with specific antibodies to confirm a protein's cellular localization. In situ hybridization studies can also be initiated to determine whether the protein and its corresponding mRNA are co-localized (88).

A particular strength of our approach is that we obtain a precise relationship between each experimental animal's metabolic phenotype and its tissue-specific protein profiles and morphological features. Hence, we prefer not to pool tissue samples.

2-D Gel Electrophoresis: Two-dimensional gel-electrophoresis (2-D gel electrophoresis) is a superior technique for the simultaneous resolution of hundreds of proteins from complex mixtures such as the insulin-sensitive tissues of diabetic mice or those susceptible hyperglycemia-induced complications.

Proteins separated by 2-D gel electrophoresis are easily visualized using colloidal Coomassie brilliant blue, or silver stain. However, Coomassie lacks detection sensitivity (˜8-10 ng; 102) and silver stain exhibits a nonlinear dynamic range of detection (103). Typically, we use the fluorescent dye SYPRO Orange (optimal excitation wavelength=470 nm; Molecular Probes), which is sensitive (0.5-10 ng detection limit), maintains a linear response over several orders of magnitude and is compatible with mass spectrometry and protein sequencing (104).

The focus of these experiments is the molecular and structural correlates of obesity and diabetes in the insulin-sensitive tissues, tissues susceptible to hyperglycemia-related damage, and serum. To date we have performed two-dimensional gel electrophoresis on distinct subcellular fractions or whole tissue lysates for several tissues at many time points during the onset of obesity and type 2 diabetes. We have achieved excellent resolution of a broad spectrum of proteins expressed at particular times during the onset and progression of obesity and diabetes.

Modified procedures may be used as needed to enrich samples for a particular protein to increase resolution. For example, serum contains labundant proteins like albumin and immunoglobulins, and it may be desirable to remove them to improve visualization of less abundant proteins. However, modifications to these abundant proteins may be significant, like the hyperglycemia-induced modifications to hemoglobin (HbA1c). In some cases, it may be desirable to modify the conditions for IEF or SDS/PAGE to enhance resolution of particular proteins. We use narrow pH IEF, and tricine or gradient-SDS-PAGE to increase resolution of particular proteins.

Mass Spec Database selection: NCBI was used as a search database since it is more complete than others such as SwissProt. However, SwissProt is better annotated and faster to search.

Mass Spec Analytical Results:

### CO2

The spot is identified as Apolipoprotein because:

It is in the same charge train of A02 which is Apolipoprotein

Both Mascot MS and MSFIT identify it as Apolipoprotein

A duplicate of this spot also yield the same ID for the spot albeit with the same low scores

Note that the Mascot MS/MS data neither confirmed the apolipoprotein identification nor pointed strongly toward an alternative identification (e.g., another protein having high scores for several fragment matches).

### E02, E05, G05 and C23

All these spots are identified as alpha-2- macroglobulin because:

The spot identified is a C-terminus fragment of a much larger protein and hence the scores are very low.

Only a couple of the fragments can be identified and these are positioned in the fragment that we identified

### A14

The spot is identified as Contrapsin because:

This Spot had low MS scores but we were able to show that 4 of a total of six peaks that were analyzed identified this spot to be contrapsin

### E17

This spot was identified as Contrapsin because

The same spot from a different gel was also identified as Contrapsin

3 MS/MS peaks were identified as contrapsin

### G23

This spot was identified as Fibrinogen because:

It has a low MS score because it is a fragment

The identification was confirmed by 2 MS/MS peaks both of which had good scores.

The MS-Fit data was considered secondary to the Mascot MS data, hence it was not relied on for identification purposes if none of the top 50 MS-FIT scorers were mouse proteins with high Mascot MS scores. It merely performed a confirmatory role.

Conclusion: We have identified tissue-specific proteins whose timing and pattern of expression correlates with the magnitude and duration of obesity and diabetes. Our results provide insight about the molecular correlates of the onset and progression of obesity and diabetes and identify novel targets for the diagnosis and/or treatment of obesity and diabetes before the onset of irreversible consequences.

### Example 2

### Reversal Experiments

An important objective of our studies is to distinguish between the reversible and irreversible consequences of diet-induced obesity and diabetes. In reversal experiments, mice that are hyperinsulinemic/hyperglycemic as a result of the high-fat diet were returned to the control diet with 10% kcal fat (Research Diets #D12450B) and monitored in accordance with the protocols described above. The experiments commenced after prolonged exposure (4 months). Typically, the animals will have been diabetic for at least 2 months.

Two-dimensional gel electrophoresis and spot analysis will be carried out essentially as described in Example 1.

### Example 3

We also monitored circulating levels of white adipose tissue (WAT)-specific proteins leptin and adiponectin (also called Acrp30, adipocyte complement related protein 30 kDa) because they are important barometers of obesity. Secretion of leptin is proportional to the body's energy stores in fat depots and it signals to the brain to reduce food intake (34,36,37). Adiponectin gene expression is induced during adipocyte differentiation and its secretion is stimulated by insulin. Adiponectin appears to increase tissue sensitivity to insulin.

In a separate set of experiments from those set forth in Example 1, but following a similar protocol, differential expression of leptin and adiponectin was studied The abundance of protein “spots” corresponding to leptin was increased in the serum isolated from a C57BL/6J mouse fed a high-fat diet compared to serum from an age-matched mouse fed a control diet. Adiponectin exhibited the same pattern. The serum levels are assumed to be indicative of differential expression in the WAT. Leptin and adiponectin serum concentrations were determined using the Crystal Chem. Inc. mouse leptin ELISA kit (#90030) and the B-Bridge International, Inc. Mouse/Rat Adiponectin ELISA kit (#K1002-1), respectively.

### Introduction to Master Tables.

The master tables reflect applicants' analysis of the proteomics data.

Master Tables 101-103 correlate each differentially expressed gel spot with one or more mouse proteins, using Mascot MS (Master Table 101), Mascot MS/MS of up to six protein fragments (Master Table 102), and/or MSFIT (Master Table 103).

These tables have the following format:

### Master Table 101: Mascot MS Matches

Col. 1: Well Number (identifies the gel spot).
Col. 2: Apparent molecular weight (kDa).

### Col. 3: Apparent pI

Col. 4: # of samples
Col. 5: Behavior (see discussion of “Behavior” in Master Table 1, below).
Col. 6: Accession # of matched mouse protein in sequence database.
Col. 7: Name of matched mouse protein in sequence database.
Col. 8: Calculated molecular weight (Da) of aligned mouse protein.
Col. 9: Calculated pI of matched mouse protein.
Col. 10: Match Score (Mascot MS implementation of Probability-based MOWSE score). Higher number is better.
Col. 11: E value of match. Lower number (i.e., more negative exponent) is better.
Col. 12: # of matched peaks, expressed in form X:Y, where Y is the total number of mass spectrometry peaks for the analyzed protein, and X is the number which could be matched to a predicted peptide fragment of the database mouse protein.
Col. 13: % covered. The percentage of the matched protein which corresponds to the predicted peptide fragments with the matched mass peaks.

For each spot there is just one entry in columns 1-5. However, there can be more than one matched mouse protein, and each will have a set of values in cols. 6-13.

### Master Table 102: Mascot MS/MS Matches

Col. 1: well number.
Col. 2: fragment size (Da).
Col. 3: Accession # of matched mouse protein in sequence database.
Col. 4: Name of matched mouse protein in sequence database.
Col. 5: Match score. (Mascot MS/MS implementation of Probability based MOWSE score).
Col. 6: E value of match.

The well number links this table to Master Table 101. Up to six fragments can be listed for a single well (gel sample). For each fragment, one or more matched mouse proteins are listed.

### Master Table 103: MSFIT Matches

Col. 1: well number
Col. 2: Apparent molecular weight

### Col. 3: Apparent pI

Col. 4: Accession # of matched mouse protein in sequence database.
Col. 5: Name of matched mouse protein in sequence database.
Col. 6: Calculated molecular weight of matched mouse protein.
Col. 7: Calculated pI of matched mouse protein.
Col. 8: MOWSE score of match. (MS-FIT implementation of Probability-based MOWSE score). Higher number is better.

Col. 9: Number of matched peaks: Number of Total Peaks.

Col. 10: % Covered.

The well number links this table to Master Tables 101 and 102. For each gel spot, one or more matched mouse proteins are listed.

### Master Table 1: Homologous Human Proteins Aligned by BlastP

For each differentially expressed mouse protein identified by Master Tables 101-103, Master Table 1 identifies:

Cols. 1-3: The mouse protein database accession #. The choice of column indicates the source of the mouse protein identification, as follows: col. 1 (Mascot MS), col. 2 (Mascot MS/MS), and col. 3 (MSFIT). The accession # acts as the link between Master Tables 101-103 and Master Table 1.
Col. 4: The behavior (differential expression) observed for the mouse protein. This column identifies the protein as favorable(F) or unfavorable (U) on the basis of its differential behavior. There are three possible comparisons, HI-D, C-HI, and C-D, where C=control (normal), HI=hyperinsulinemic, and D=diabetic. If HI>D, C>HI, or C>D, the behavior for that subject comparison is considered unfavorable. If the inequality is reversed, the behavior for that subject comparison is considered favorable.

In the Master Table, the numerical value is the ratio of the greater value to the lesser value.

If this ratio is at least two fold, the degree of differential expression is considered strong. Usually only mouse proteins exhibiting at least one strong differential expression behavior are listed in the Master Table; exceptions are noted in the Examples.

In some of the related applications cited above, and perhaps occasionally in this application, a ratio may be given as a negative number. This does not have its usual mathematical meaning; it is merely a flag that in the comparison, the former value was less than the latter one, i.e., the gene was favorable. For the purpose of applying the teachings of the specification concerning desired ratios, any negative value should be converted to a positive one by taking its absolute value.

If the behavior of the protein is mixed, then either the individual favorable and unfavorable behaviors are listed, with the degree of differential expression stated, or the behavior is imply labeled as mixed (M), with no stated degree.

Col. 5: A related human protein, identified by its database accession number. Usually, several such proteins are identified relative to each mouse protein. These proteins have been identified by BLASTP searches.
Col. 6: The name of the related human protein.
Col. 7: The score (in bits) for the alignment performed by the BLASTP program.
Col. 8: The E-value for the alignment performed by the BLASTP program

Unless otherwise indicated, the bit score and E-value for the alignment is with respect to the alignment of the mouse protein of cols. 1, 2 or 3 to the human protein of col. 5 by BlastP, according to the default parameters.

Master Table 1 is divided into three subtables on the basis of the behavior in col. 4. If a protein has at least one significantly favorable behavior, and no significantly unfavorable ones, it is put into Subtable 1A. In the opposite case, it is put into Subtable 1B. If its behavior is mixed, i.e., at least one significantly favorable and at least one significantly unfavorable, it is put into Subtable 1C. Note that this classification is based on the strongest observed differential expression behaviors for each of the three subject comparisons, C-HI, HI-D and C-D.

MASTER TABLE 1
Human Protein Homologues of Mouse Proteins Differentially Expressed in Serum of
Control(C)/Hyperinsulinemic(HI)/Diabetic(D) Mice

| | | | | | | | |
|---|---|---|---|---|---|---|---|
| Mouse | | | | | | | |
| Protein | | | | | | | |
| Accession | | | | | | | |
| No. | | | | Human | | | |
| Mascot | Mascot | | | Homolog | | Score | |
| MS | MS/MS | MSFIT | Behavior | Accession | Human Protein Name | Bits | e-value |

Subtable 1A: Favorable Human Proteins/Favorable Mouse Proteins

| | | | | | | | |
|---|---|---|---|---|---|---|---|
| Q8BPD5 | | | F: (C-D) | AAA35545 | proapo-A-I protein | 264 | 1.00e−70 |
| | | | 21.9 | | | | |
| | | | | AAQ91811 | apolipoprotein A-I [Homo sapiens]. | 263 | 2.00e−70 |
| | | | | AAA51747 | proapolipoprotein | 252 | 6.00e−67 |
| | | | | 750843A | protein, lipid binding AI | 216 | 3.00e−56 |
| | | | | 1AV1_D | Chain D, Crystal Structure Of Human | 202 | 4.00e−52 |
| | | | | | Apolipoprotein A-I. | | |
| | | | | AAB22835 | apolipoprotein AI, apo AI [human, spleen, Peptide | 88 | 2.00e−17 |
| | | | | | Mutant, 88 aa]. | | |
| | | Q00623 | | AAQ91811 | apolipoprotein A-I [Homo sapiens]. | 310 | 1.00e−84 |
| | | | | AAA35545 | proapo-A-I protein | 310 | 2.00e−84 |
| | | | | AAA51747 | proapolipoprotein | 301 | 8.00e−82 |
| | | | | 750843A | protein, lipid binding AI | 260 | 2.00e−69 |
| | | | | 1AV1_D | Chain D, Crystal Structure Of Human | 245 | 7.00e−65 |
| | | | | | Apolipoprotein A-I. | | |
| | | | | AAB22835 | apolipoprotein AI, apo AI [human, spleen, Peptide | 101 | 1.00e−21 |
| | | | | | Mutant, 88 aa]. | | |
| | Q8BPD5 | | | AAA35545 | proapo-A-I protein | 264 | 1.00e−70 |
| | | | | 750843A | protein, lipid binding AI | 263 | 2.00e−70 |
| | | | | AAQ91811 | apolipoprotein A-I [Homo sapiens]. | 252 | 6.00e−67 |
| | | | | AAA51747 | proapolipoprotein | 216 | 3.00e−56 |
| | | | | 1AV1_D | Chain D, Crystal Structure Of Human | 202 | 4.00e−52 |
| | | | | | Apolipoprotein A-I. | | |
| | Q8BPD5 | | | AAA35545 | proapo-A-I protein | 264 | 1.00e−70 |
| | | | | 750843A | protein, lipid binding AI | 263 | 2.00e−70 |
| | | | | AAQ91811 | apolipoprotein A-I [Homo sapiens]. | 252 | 6.00e−67 |
| | | | | AAA51747 | proapolipoprotein | 216 | 3.00e−56 |
| | | | | 1AV1_D | Chain D, Crystal Structure Of Human | 202 | 4.00e−52 |
| | | | | | Apolipoprotein A-I. | | |
| | Q8BPD5 | | | AAA35545 | proapo-A-I protein | 264 | 1.00e−70 |
| | | | | 750843A | protein, lipid binding AI | 263 | 2.00e−70 |
| | | | | AAQ91811 | apolipoprotein A-I [Homo sapiens]. | 252 | 6.00e−67 |
| | | | | AAA51747 | proapolipoprotein | 216 | 3.00e−56 |
| | | | | 1AV1_D | Chain D, Crystal Structure Of Human | 202 | 4.00e−52 |
| | | | | | Apolipoprotein A-I. | | |
| | Q8BPD5 | | | 750843A | protein, lipid binding AI | 264 | 1.00e−70 |
| | | | | AAQ91811 | apolipoprotein A-I [Homo sapiens]. | 263 | 2.00e−70 |
| | | | | AAA51747 | proapolipoprotein | 252 | 6.00e−67 |
| | | | | 1AV1_D | Chain D, Crystal Structure Of Human | 216 | 3.00e−56 |
| | | | | | Apolipoprotein A-I. | | |
| | | | | AAA35545 | proapo-A-I protein | 202 | 4.00e−52 |
| Q8BPD5 | | | F: (C-D) | AAA35545 | proapo-A-I protein | 264 | 1.00e−70 |
| | | | 16.1 | | | | |
| | | | | AAQ91811 | apolipoprotein A-I [Homo sapiens]. | 263 | 2.00e−70 |
| | | | | AAA51747 | proapolipoprotein | 252 | 6.00e−67 |
| | | | | 750843A | protein, lipid binding AI | 216 | 3.00e−56 |
| | | | | 1AV1_D | Chain D, Crystal Structure Of Human | 202 | 4.00e−52 |
| | | | | | Apolipoprotein A-I. | | |
| | | | | AAB22835 | apolipoprotein AI, apo AI [human, spleen, Peptide | 88 | 2.00e−17 |
| | | | | | Mutant, 88 aa]. | | |
| | | Q00623 | | AAQ91811 | apolipoprotein A-I [Homo sapiens]. | 310 | 1.00e−84 |
| | | | | AAA35545 | proapo-A-I protein | 310 | 2.00e−84 |
| | | | | AAA51747 | proapolipoprotein | 301 | 8.00e−82 |
| | | | | 750843A | protein, lipid binding AI | 260 | 2.00e−69 |
| | | | | 1AV1_D | Chain D, Crystal Structure Of Human | 245 | 7.00e−65 |
| | | | | | Apolipoprotein A-I. | | |
| | | | | AAB22835 | apolipoprotein AI, apo AI [human, spleen, Peptide | 101 | 1.00e−21 |
| | | | | | Mutant, 88 aa]. | | |
| | Q8BPD5 | | | AAA35545 | proapo-A-I protein | 264 | 1.00e−70 |
| | | | | AAA51747 | proapolipoprotein | 263 | 2.00e−70 |
| | | | | AAQ91811 | apolipoprotein A-I [Homo sapiens]. | 252 | 6.00e−67 |
| | | | | 750843A | protein, lipid binding AI | 216 | 3.00e−56 |
| | | | | 1AV1_D | Chain D, Crystal Structure Of Human | 202 | 4.00e−52 |
| | | | | | Apolipoprotein A-I. | | |
| | | | | AAB22835 | apolipoprotein AI, apo AI [human, spleen, Peptide | 88 | 2.00e−17 |
| | | | | | Mutant, 88 aa]. | | |
| | Q8BPD5 | | | AAA35545 | proapo-A-I protein | 264 | 1.00e−70 |
| | | | | AAA51747 | proapolipoprotein | 263 | 2.00e−70 |
| | | | | AAQ91811 | apolipoprotein A-I [Homo sapiens]. | 252 | 6.00e−67 |
| | | | | 750843A | protein, lipid binding AI | 216 | 3.00e−56 |
| | | | | 1AV1_D | Chain D, Crystal Structure Of Human Apolipoprotein | 202 | 4.00e−52 |
| | | | | | A-I. | | |
| | | | | AAB22835 | apolipoprotein AI, apo AI [human, spleen, Peptide | 88 | 2.00e−17 |
| | | | | | Mutant, 88 aa]. | | |
| | Q8BPD5 | | | AAA35545 | proapo-A-I protein | 264 | 1.00e−70 |
| | | | | AAA51747 | proapolipoprotein | 263 | 2.00e−70 |
| | | | | AAQ91811 | apolipoprotein A-I [Homo sapiens]. | 252 | 6.00e−67 |
| | | | | 750843A | protein, lipid binding AI | 216 | 3.00e−56 |
| | | | | 1AV1_D | Chain D, Crystal Structure Of Human Apolipoprotein | 202 | 4.00e−52 |
| | | | | | A-I. | | |
| | | | | AAB22835 | apolipoprotein AI, apo AI [human, spleen, Peptide | 88 | 2.00e−17 |
| | | | | | Mutant, 88 aa]. | | |
| | Q8BPD5 | | | AAA35545 | proapo-A-I protein | 264 | 1.00e−70 |
| | | | | AAA51747 | proapolipoprotein | 263 | 2.00e−70 |
| | | | | AAQ91811 | apolipoprotein A-I [Homo sapiens]. | 252 | 6.00e−67 |
| | | | | 750843A | protein, lipid binding AI | 216 | 3.00e−56 |
| | | | | 1AV1_D | Chain D, Crystal Structure Of Human Apolipoprotein | 202 | 4.00e−52 |
| | | | | | A-I. | | |
| | | | | AAB22835 | apolipoprotein AI, apo AI [human, spleen, Peptide | 88 | 2.00e−17 |
| | | | | | Mutant, 88 aa]. | | |
| AAH57983 | | | F: (C-D) | 1009174A | macroglobulin alpha2 | 1573 | 0 |
| | | | 2.3 | | | | |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| | AAH57983 | | | 1009174A | macroglobulin alpha2 | 1573 | 0 |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| | | | | | | | 0 |
| Q91ZP1 | | | F: (C-D) | AAA52429 | beta-fibrinogen precursor | 469 | e−132 |
| | | | 2.1 | | | | |
| | | | | AAA18024 | fibrinogen beta chain [Homo sapiens]. | 469 | e−132 |
| | | | | NP_005132 | fibrinogen, beta chain preproprotein [Homo sapiens]. | 469 | e−132 |
| | | | | 0401173A | fibrin beta. | 466 | e−131 |
| | Q91ZP1 | | | AAA52429 | beta-fibrinogen precursor | 469 | e−132 |
| | | | | AAA18024 | fibrinogen beta chain [Homo sapiens]. | 469 | e−132 |
| | | | | NP_005132 | fibrinogen, beta chain preproprotein [Homo sapiens]. | 469 | e−132 |
| | | | | 0401173A | fibrin beta. | 466 | e−131 |
| | Q8K0E8 | | | AAA18024 | fibrinogen beta chain [Homo sapiens]. | 823 | 0 |
| | | | | AAA52429 | beta-fibrinogen precursor | 820 | 0 |
| | | | | NP_005132 | fibrinogen, beta chain preproprotein [Homo sapiens]. | 820 | 0 |
| | | | | 0401173A | fibrin beta. | 799 | 0 |
| JC1237 | | | F: (C-D) | AAA35545 | proapo-A-I protein | 263 | 2.00e−70 |
| | | | 2.5 | | | | |
| | | | | AAA51747 | proapolipoprotein. | 251 | 1.00e−66 |
| | | | | 750843A | protein, lipid binding AI | 218 | 9.00e−57 |
| | | | | 1AV1_D | Chain D, Crystal Structure Of Human Apolipoprotein | 201 | 9.00e−52 |
| | | | | | A-I. | | |
| | JC1237 | | | AAA35545 | proapo-A-I protein | 263 | 2.00e−70 |
| | | | | AAA51747 | proapolipoprotein. | 251 | 1.00e−66 |
| | | | | 750843A | protein, lipid binding AI | 218 | 9.00e−57 |
| | | | | 1AV1_D | Chain D, Crystal Structure Of Human Apolipoprotein | 201 | 9.00e−52 |
| | | | | | A-I. | | |
| | Q8BPD5 | | | AAA35545 | proapo-A-I protein | 264 | 1.00e−70 |
| | | | | AAA51747 | proapolipoprotein. | 252 | 6.00e−67 |
| | | | | 750843A | protein, lipid binding AI | 216 | 3.00e−56 |
| | | | | 1AV1_D | Chain D, Crystal Structure Of Human Apolipoprotein | 202 | 4.00e−52 |
| | | | | | A-I. | | |
| JU0036 | | | F: (C-D) | 1506383A | apolipoprotein E mutant E3K | 314 | 2.00e−85 |
| | | | 2.5 | | | | |
| | | | | AAB59397 | apolipoprotein E. | 313 | 2.00e−85 |
| | | | | NP_000032 | apolipoprotein E precursor [Homo sapiens]. | 313 | 2.00e−85 |
| | | | | AAB59518 | apolipoprotein E | 312 | 6.00e−85 |
| | JU0036 | | | 1506383A | apolipoprotein E mutant E3K | 314 | 2.00e−85 |
| | | | | AAB59397 | apolipoprotein E. | 313 | 2.00e−85 |
| | | | | NP_000032 | apolipoprotein E precursor [Homo sapiens]. | 313 | 2.00e−85 |
| | | | | AAB59518 | apolipoprotein E | 312 | 6.00e−85 |
| | JU0036 | | | 1506383A | apolipoprotein E mutant E3K | 314 | 2.00e−85 |
| | | | | AAB59397 | apolipoprotein E. | 313 | 2.00e−85 |
| | | | | NP_000032 | apolipoprotein E precursor [Homo sapiens]. | 313 | 2.00e−85 |
| | | | | AAB59518 | apolipoprotein E | 312 | 6.00e−85 |
| | JU0036 | | | 1506383A | apolipoprotein E mutant E3K | 314 | 2.00e−85 |
| | | | | AAB59397 | apolipoprotein E. | 313 | 2.00e−85 |
| | | | | NP_000032 | apolipoprotein E precursor [Homo sapiens]. | 313 | 2.00e−85 |
| | | | | AAB59518 | apolipoprotein E | 312 | 6.00e−85 |
| JU0036 | | | F: (C-D) | 1506383A | apolipoprotein E mutant E3K | 314 | 2.00e−85 |
| | | | 3.3 | | | | |
| | | | | AAB59397 | apolipoprotein E. | 313 | 2.00e−85 |
| | | | | NP_000032 | apolipoprotein E precursor [Homo sapiens]. | 313 | 2.00e−85 |
| | | | | AAB59518 | apolipoprotein E | 312 | 6.00e−85 |
| | JU0036 | | | 1506383A | apolipoprotein E mutant E3K | 314 | 2.00e−85 |
| | | | | AAB59397 | apolipoprotein E. | 313 | 2.00e−85 |
| | | | | NP_000032 | apolipoprotein E precursor [Homo sapiens]. | 313 | 2.00e−85 |
| | | | | AAB59518 | apolipoprotein E | 312 | 6.00e−85 |
| | JU0036 | | | 1506383A | apolipoprotein E mutant E3K | 314 | 2.00e−85 |
| | | | | AAB59397 | apolipoprotein E. | 313 | 2.00e−85 |
| | | | | NP_000032 | apolipoprotein E precursor [Homo sapiens]. | 313 | 2.00e−85 |
| | | | | AAB59518 | apolipoprotein E | 312 | 6.00e−85 |

Subtable 1B: Unfavorable Human Proteins/Unfavorable Mouse Proteins

| | | | | | | | |
|---|---|---|---|---|---|---|---|
| B40892 | | | U: (C-D) | P06727 | Apolipoprotein A-IV precursor (Apo-AIV). | 444 | e−124 |
| | | | 6.3 | | | | |
| | | | | AAQ91809 | apolipoprotein A-IV [Homo sapiens]. | 443 | e−124 |
| | | | | NP_000473 | apolipoprotein A-IV precursor [Homo sapiens]. | 441 | e−123 |
| | | | | LPHUA4 | apolipoprotein A-IV precursor [validated] - human. | 439 | e−123 |
| | | | | AAA51748 | apolipoprotein A-IV precursor. | 433 | e−121 |
| | Q81017 | | | P06727 | Apolipoprotein A-IV precursor (Apo-AIV). | 374 | e−103 |
| | | | | AAQ91809 | apolipoprotein A-IV [Homo sapiens]. | 374 | e−103 |
| | | | | NP_000473 | apolipoprotein A-IV precursor [Homo sapiens]. | 373 | e−103 |
| | | | | LPHUA4 | apolipoprotein A-IV precursor [validated] - human. | 369 | e−102 |
| | A25281 | | | P06727 | Apolipoprotein A-IV precursor (Apo-AIV). | 367 | e−101 |
| | | | | AAQ91809 | apolipoprotein A-IV [Homo sapiens]. | 367 | e−101 |
| | | | | NP_000473 | apolipoprotein A-IV precursor [Homo sapiens]. | 367 | e−101 |
| | | | | LPHUA4 | apolipoprotein A-IV precursor [validated] - human. | 363 | e−100 |
| | | | | AAA51748 | apolipoprotein A-IV precursor. | 356 | 4.00e−98 |
| | | | | AAB59516 | apolipoprotein A-IV | 228 | 1.00e−59 |
| | | B40892 | | P06727 | Apolipoprotein A-IV precursor (Apo-AIV). | 444 | e−124 |
| | | | | AAQ91809 | apolipoprotein A-IV [Homo sapiens]. | 443 | e−124 |
| | | | | NP_000473 | apolipoprotein A-IV precursor [Homo sapiens]. | 441 | e−123 |
| | | | | LPHUA4 | apolipoprotein A-IV precursor [validated] - human. | 439 | e−123 |
| | | | | AAA51748 | apolipoprotein A-IV precursor. | 433 | e−121 |
| Q81017 | | | U: (C-D) | P06727 | Apolipoprotein A-IV precursor (Apo-AIV). | 374 | e−103 |
| | | | 11.5 | | | | |
| | | | | AAQ91809 | apolipoprotein A-IV [Homo sapiens]. | 374 | e−103 |
| | | | | NP_000473 | apolipoprotein A-IV precursor [Homo sapiens]. | 373 | e−103 |
| | | | | LPHUA4 | apolipoprotein A-IV precursor [validated] - human. | 369 | e−102 |
| | | B40892 | | P06727 | Apolipoprotein A-IV precursor (Apo-AIV). | 444 | e−124 |
| | | | | AAQ91809 | apolipoprotein A-IV [Homo sapiens]. | 443 | e−124 |
| | | | | NP_000473 | apolipoprotein A-IV precursor [Homo sapiens]. | 441 | e−123 |
| | | | | LPHUA4 | apolipoprotein A-IV precursor [validated] - human. | 439 | e−123 |
| | | | | AAA51748 | apolipoprotein A-IV precursor. | 433 | e−121 |
| Q62257 | | | U: (C-D) | CAA48671 | alpha1-antichymotrypsin [Homo sapiens]. | 402 | e−112 |
| | | | 3.8 | | | | |
| | | | | AAD08810 | alpha-1-antichymotrypsin precursor [Homo sapiens]. | 401 | e−111 |
| | | | | NP_001076 | serine (or cysteine) proteinase inhibitor, clade A, | 400 | e−111 |
| | | | | AAH34554 | Serine (or cysteine) proteinase inhibitor, clade A, | 400 | e−111 |
| | | | | ITHUC | alpha-1-antichymotrypsin precursor - human. | 394 | e−109 |
| | | | | AAA51560 | alpha-1-antichymotrypsin precursor. | 391 | e−108 |
| | | | | 1QMN_A | Chain A, Alpha1-Antichymotrypsin Serpin | 385 | e−106 |
| | | Q8VCH3 | | NP_001076 | serine (or cysteine) proteinase inhibitor, clade A, | 417 | e−116 |
| | | | | AAH34554 | Serine (or cysteine) proteinase inhibitor, clade A, | 416 | e−116 |
| | | | | CAA48671 | alpha1-antichymotrypsin [Homo sapiens]. | 416 | e−116 |
| | | | | AAD08810 | alpha-1-antichymotrypsin precursor [Homo sapiens]. | 410 | e−114 |
| | | | | ITHUC | alpha-1-antichymotrypsin precursor - human. | 409 | e−114 |
| | | | | AAA51560 | alpha-1-antichymotrypsin precursor. | 401 | e−111 |
| | | | | 1QMN_A | Chain A, Alpha1-Antichymotrypsin Serpin | 395 | e−110 |
| | Q62257 | | | CAA48671 | alpha1-antichymotrypsin [Homo sapiens]. | 402 | e−112 |
| | | | | AAD08810 | alpha-1-antichymotrypsin precursor [Homo sapiens]. | 401 | e−111 |
| | | | | NP_001076 | serine (or cysteine) proteinase inhibitor, clade A, | 400 | e−111 |
| | | | | AAH34554 | Serine (or cysteine) proteinase inhibitor, clade A, | 400 | e−111 |
| | | | | ITHUC | alpha-1-antichymotrypsin precursor - human. | 394 | e−109 |
| | | | | AAA51560 | alpha-1-antichymotrypsin precursor. | 391 | e−108 |
| | | | | 1QMN_A | Chain A, Alpha1-Antichymotrypsin Serpin | 385 | e−106 |
| | Q62257 | | | CAA48671 | alpha1-antichymotrypsin [Homo sapiens]. | 402 | e−112 |
| | | | | AAD08810 | alpha-1-antichymotrypsin precursor [Homo sapiens]. | 401 | e−111 |
| | | | | NP_001076 | serine (or cysteine) proteinase inhibitor, clade A, | 400 | e−111 |
| | | | | AAH34554 | Serine (or cysteine) proteinase inhibitor, clade A, | 400 | e−111 |
| | | | | ITHUC | alpha-1-antichymotrypsin precursor - human. | 394 | e−109 |
| | | | | AAA51560 | alpha-1-antichymotrypsin precursor. | 391 | e−108 |
| | | | | 1QMN_A | Chain A, Alpha1-Antichymotrypsin Serpin | 385 | e−106 |
| | Q62257 | | | CAA48671 | alpha1-antichymotrypsin [Homo sapiens]. | 402 | e−112 |
| | | | | AAD08810 | alpha-1-antichymotrypsin precursor [Homo sapiens]. | 401 | e−111 |
| | | | | NP_001076 | serine (or cysteine) proteinase inhibitor, clade A, | 400 | e−111 |
| | | | | AAH34554 | Serine (or cysteine) proteinase inhibitor, clade A, | 400 | e−111 |
| | | | | ITHUC | alpha-1-antichymotrypsin precursor - human. | 394 | e−109 |
| | | | | AAA51560 | alpha-1-antichymotrypsin precursor. | 391 | e−108 |
| | | | | 1QMN_A | Chain A, Alpha1-Antichymotrypsin Serpin | 385 | e−106 |
| I49471 | | | U: (C-D) | AAA51547 | alpha-1-antitrypsin precursor | 512 | e−145 |
| | | | 8.6 | | | | |
| | | | | AAV38262 | serine (or cysteine) proteinase inhibitor, clade A | 511 | e−145 |
| | | | | NP_001002236 | serine (or cysteine) proteinase inhibitor, clade A | 510 | e−144 |
| | | | | AAH11991 | Serine (or cysteine) proteinase inhibitor, clade A | 509 | e−144 |
| | | | | 1012287A | antitrypsin alpha1 mutant | 509 | e−144 |
| | | | | AAA51546 | alpha-1-antitrypsin | 508 | e−144 |
| | | | | AAB59495 | alpha-1-antitrypsin | 508 | e−144 |
| | | | | AAF29581 | PRO0684 [Homo sapiens]. | 508 | e−144 |
| | | | | 1KCT | Alpha1-Antitrypsin | 489 | e−138 |
| | | P08226 | | AAV38262 | serine (or cysteine) proteinase inhibitor, clade A | 518 | e−147 |
| | | | | AAA51547 | alpha-1-antitrypsin precursor | 518 | e−147 |
| | | | | NP_001002236 | serine (or cysteine) proteinase inhibitor, clade A | 517 | e−146 |
| | | | | 1012287A | antitrypsin alpha1 mutant | 517 | e−146 |
| | | | | AAH11991 | Serine (or cysteine) proteinase inhibitor, clade A | 516 | e−146 |
| | | | | AAA51546 | alpha-1-antitrypsin | 515 | e−146 |
| | | | | AAF29581 | PRO0684 [Homo sapiens]. | 514 | e−145 |
| | | | | AAB59495 | alpha-1-antitrypsin | 514 | e−145 |
| | I49471 | | | AAA51547 | alpha-1-antitrypsin precursor | 512 | e−145 |
| | | | | AAV38262 | serine (or cysteine) proteinase inhibitor, clade A | 511 | e−145 |
| | | | | NP_001002236 | serine (or cysteine) proteinase inhibitor, clade A | 510 | e−144 |
| | | | | AAH11991 | Serine (or cysteine) proteinase inhibitor, clade A | 509 | e−144 |
| | | | | 1012287A | antitrypsin alpha1 mutant | 509 | e−144 |
| | | | | AAA51546 | alpha-1-antitrypsin | 508 | e−144 |
| | | | | AAB59495 | alpha-1-antitrypsin | 508 | e−144 |
| | | | | AAF29581 | PRO0684 [Homo sapiens]. | 508 | e−144 |
| | | | | 1KCT | Alpha1-Antitrypsin | 489 | e−138 |
| | I49471 | | | AAA51547 | alpha-1-antitrypsin precursor | 512 | e−145 |
| | | | | AAV38262 | serine (or cysteine) proteinase inhibitor, clade A | 511 | e−145 |
| | | | | NP_001002236 | serine (or cysteine) proteinase inhibitor, clade A | 510 | e−144 |
| | | | | AAH11991 | Serine (or cysteine) proteinase inhibitor, clade A | 509 | e−144 |
| | | | | 1012287A | antitrypsin alpha1 mutant | 509 | e−144 |
| | | | | AAA51546 | alpha-1-antitrypsin | 508 | e−144 |
| | | | | AAB59495 | alpha-1-antitrypsin | 508 | e−144 |
| | | | | AAF29581 | PRO0684 [Homo sapiens]. | 508 | e−144 |
| | | | | 1KCT | Alpha1-Antitrypsin | 489 | e−138 |
| JU0036 | | | U: (C-D) | 1506383A | apolipoprotein E mutant E3K | 314 | 2.00e−85 |
| | | | 3.2 | | | | |
| | | | | AAB59397 | apolipoprotein E. | 313 | 2.00e−85 |
| | | | | NP_000032 | apolipoprotein E precursor [Homo sapiens]. | 313 | 2.00e−85 |
| | | | | AAB59518 | apolipoprotein E | 312 | 6.00e−85 |
| | | P08226 | | AAB59397 | apolipoprotein E. | 427 | e−119 |
| | | | | NP_000032 | apolipoprotein E precursor [Homo sapiens]. | 424 | e−118 |
| | | | | 1506383A | apolipoprotein E mutant E3K | 424 | e−118 |
| | | | | AAB59518 | apolipoprotein E | 421 | e−117 |

Subtable 1C: Mixed Human Proteins/Mixed Mouse Proteins

| | | | | | | | |
|---|---|---|---|---|---|---|---|
| AAH57983 | | | M: (C-D) | 1009174A | macroglobulin alpha2 | 1573 | 0 |
| | | | U: 4 wks | | | | |
| | | | 5.9 | | | | |
| | | | F: 8 wks | | | | |
| | | | 4.8 | | | | |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| | S27001 | | | 1009174A | macroglobulin alpha2 | 1573 | 0 |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| | AAH57983 | | | 1009174A | macroglobulin alpha2 | 1573 | 0 |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| | AAH57983 | | | 1009174A | macroglobulin alpha2 | 1573 | 0 |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| AAH57983 | | | M: (C-D) | 1009174A | macroglobulin alpha2 | 1573 | 0 |
| | | | U: 4 wks | | | | |
| | | | 5.9 | | | | |
| | | | F: 8 wks | | | | |
| | | | 4.8 | | | | |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| | AAH57983 | | | 1009174A | macroglobulin alpha2 | 1573 | 0 |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| | S27001 | | | 1009174A | macroglobulin alpha2 | 1573 | 0 |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| | S27001 | | | 1009174A | macroglobulin alpha2 | 1573 | 0 |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| | AAH57983 | | | 1009174A | macroglobulin alpha2 | 1573 | 0 |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| S27001 | | | M: (C-D) | 1009174A | macroglobulin alpha2 | 1573 | 0 |
| | | | U: 4 wks | | | | |
| | | | 2.7 | | | | |
| | | | F: 8 wks | | | | |
| | | | 2.6 | | | | |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| | AAH57983 | | | 1009174A | macroglobulin alpha2 | 1573 | 0 |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| | S27001 | | | 1009174A | macroglobulin alpha2 | 1573 | 0 |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| | S27001 | | | 1009174A | macroglobulin alpha2 | 1573 | 0 |
| | | | | AAT02228 | alpha 2 macroglabulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Hamo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| | S27001 | | | 1009174A | macroglobulin alpha2 | 1573 | 0 |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| | S27001 | | | 1009174A | macroglobulin alpha2 | 1573 | 0 |
| | | | | AAT02228 | alpha 2 macroglobulin [Homo sapiens]. | 1580 | 0 |
| | | | | CAH18188 | hypothetical protein [Homo sapiens]. | 1585 | 0 |
| | | | | NP_000005 | alpha-2-macroglobulin precursor [Homo sapiens]. | 1584 | 0 |
| | | | | NP_002855 | pregnancy-zone protein [Homo sapiens]. | 1520 | 0 |
| | | | | XP_495917 | ovostatin 2 [Homo sapiens]. | 853 | 0 |
| | | | | CAE51409 | ovostatin-2 [Homo sapiens]. | 853 | 0 |
| | | | | AAA51552 | alpha-2-macroglobulin. | 828 | 0 |
| | | | | BAC85654 | unnamed protein product [Homo sapiens | 717 | 0 |
| Q9CY54 | | | M: (C-D) | AAN11320 | hemoglobin beta chain variant Hb S-Wake | 242 | 1.00e−64 |
| | | | F: 4 wks | | [Homo sapiens]. | | |
| | | | 2.28 | | | | |
| | | | U: 8 wks | | | | |
| | | | 3.4 | | | | |
| | | | | AAN84548 | beta globin chain variant [Homo sapiens]. | 240 | 5.00e−64 |
| | | | | AAL68978 | mutant beta-globin [Homo sapiens]. | 240 | 6.00e−64 |
| | | | | AAF00489 | hemoglobin beta subunit variant [Homo sapiens]. | 239 | 9.00e−64 |
| | | | | CAA23759 | unnamed protein product [Homo sapiens]. | 239 | 9.00e−64 |
| | | | | 1R1Y_D | Chain D, Crystal Structure Of Deoxy-Human | 239 | 1.00e−63 |
| | | | | | Hemoglobin | | |
| | | | | 1NEJ_D | Chain D, Crystalline Human Carbonmonoxy | 239 | 1.00e−63 |
| | | | | | Hemoglobin S | | |
| | | | | AAD19696 | hemoglobin beta chain [Homo sapiens]. | 239 | 1.00e−63 |
| | | | | 1M9P_D | Chain D, Crystalline Human Carbonmonoxy | 238 | 1.00e−63 |
| | | | | | Hemoglobin C | | |
| | | | | 6HBW_D | Chain D, Crystal Structure Of Deoxy-Human | 238 | 2.00e−63 |
| | | | | | Hemoglobin Beta6 | | |
| | | | | 1O1N_D | Chain D, Deoxy Hemoglobin | 238 | 3.00e−63 |
| | | | | 1HDB_D | Chain D, Human Hemoglobin | 238 | 3.00e−63 |
| | | P02088 | | AAN11320 | hemoglobin beta chain variant Hb S-Wake | 249 | 1.00e−66 |
| | | | | | [Homo sapiens]. | | |
| | | | | 1NEJ_D | Chain D, Crystalline Human Carbonmonoxy | 248 | 2.00e−66 |
| | | | | | Hemoglobin S | | |
| | | | | NP_000509 | beta globin [Homo sapiens]. | 248 | 3.00e−66 |
| | | | | 1R1Y_D | Chain D, Crystal Structure Of Deoxy-Human | 248 | 3.00e−66 |
| | | | | | Hemoglobin | | |
| | | | | 1M9P_D | Chain D, Crystalline Human Carbonmonoxy | 248 | 3.00e−66 |
| | | | | | Hemoglobin C | | |
| | | | | AAL68978 | mutant beta-globin [Homo sapiens]. | 247 | 4.00e−66 |
| | | | | 6HBW_D | Chain D, Crystal Structure Of Deoxy-Human | 247 | 4.00e−66 |
| | | | | | Hemoglobin Beta6 | | |
| | | | | AAN11320 | hemoglobin beta chain variant Hb S-Wake | 247 | 6.00e−66 |
| | | | | | [Homo sapiens]. | | |
| | | | | 1O1N_D | Chain D, Deoxy Hemoglobin | 247 | 6.00e−66 |
| | | | | 1GBV_D | Chain D, (Alpha-Oxy, Beta-(C112g)deoxy) T-State | 247 | 6.00e−66 |
| | | | | | Human Hemoglobin | | |
| | | | | CAA23759 | unnamed protein product [Homo sapiens]. | 246 | 7.00e−66 |
| | | | | 1NQP_D | Chain D, Crystal Structure Of Human Hemoglobin E | 246 | 7.00e−66 |
| | Q9CY54 | | | AAN11320 | hemoglobin beta chain variant Hb S-Wake | 242 | 1.00e−64 |
| | | | | | [Homo sapiens]. | | |
| | | | | AAN84548 | beta globin chain variant [Homo sapiens]. | 240 | 5.00e−64 |
| | | | | AAL68978 | mutant beta-globin [Homo sapiens]. | 240 | 6.00e−64 |
| | | | | AAF00489 | hemoglobin beta subunit variant [Homo sapiens]. | 239 | 9.00e−64 |
| | | | | CAA23759 | unnamed protein product [Homo sapiens]. | 239 | 9.00e−64 |
| | | | | 1R1Y_D | Chain D, Crystal Structure Of Deoxy-Human | 239 | 1.00e−63 |
| | | | | | Hemoglobin | | |
| | | | | 1NEJ_D | Chain D, Crystalline Human Carbonmonoxy | 239 | 1.00e−63 |
| | | | | | Hemoglobin S | | |
| | | | | AAD19696 | hemoglobin beta chain [Homo sapiens]. | 239 | 1.00e−63 |
| | | | | 1M9P_D | Chain D, Crystalline Human Carbonmonoxy | 238 | 1.00e−63 |
| | | | | | Hemoglobin C | | |
| | | | | 6HBW_D | Chain D, Crystal Structure Of Deoxy-Human | 238 | 2.00e−63 |
| | | | | | Hemoglobin Beta6 | | |
| | | | | 1O1N_D | Chain D, Deoxy Hemoglobin | 238 | 3.00e−63 |
| | | | | 1HDB_D | Chain D, Human Hemoglobin | 238 | 3.00e−63 |
| | HBMS | | | AAN11320 | hemoglobin beta chain variant Hb S-Wake | 244 | 3.00e−65 |
| | | | | | [Homo sapiens]. | | |
| | | | | AAL68978 | mutant beta-globin [Homo sapiens]. | 243 | 6.00e−65 |
| | | | | AAN84548 | beta globin chain variant [Homo sapiens]. | 243 | 8.00e−65 |
| | | | | AAF00489 | hemoglobin beta subunit variant [Homo sapiens]. | 243 | 1.00e−64 |
| | | | | CAA23759 | unnamed protein product [Homo sapiens]. | 242 | 1.00e−64 |
| | | | | 1NEJ_D | Chain D, Crystalline Human Carbonmonoxy | 242 | 2.00e−64 |
| | | | | | Hemoglobin S | | |
| | | | | 1R1Y_D | Chain D, Crystal Structure Of Deoxy-Human | 242 | 2.00e−64 |
| | | | | | Hemoglobin | | |
| | | | | AAD19696 | hemoglobin beta chain [Homo sapiens]. | 242 | 2.00e−64 |
| | | | | 1M9P_D | Chain D, Crystalline Human Carbonmonoxy | 242 | 2.00e−64 |
| | | | | | Hemoglobin C | | |
| | | | | 6HBW_D | Chain D, Crystal Structure Of Deoxy-Human | 241 | 3.00e−64 |
| | | | | | Hemoglobin Beta6 | | |
| | | | | 1O1N_D | Chain D, Deoxy Hemoglobin | 241 | 4.00e−64 |
| | | | | 1HDB_D | Chain D, Human Hemoglobin | 240 | 5.00e−64 |
| | HBMS | | | AAK37554 | hemoglobin alpha-1 globin chain [Homo sapiens]. | 227 | 6.00e−60 |
| | | | | AAH32122 | HBA2 protein [Homo sapiens]. | 225 | 2.00e−59 |
| | | | | AAN04486 | hemoglobin alpha-2 [Homo sapiens]. | 224 | 4.00e−59 |
| | | | | AAF72612 | alpha-2-globin [Homo sapiens]. | 223 | 9.00e−59 |
| | | | | 1BAB_C | Chain C, Hemoglobin Thionville Alpha Chain Mutant | 223 | 1.00e−58 |
| | | | | 1C7D_A | Chain A, Deoxy Rhb1.2 (Recombinant Hemoglobin). | 222 | 1.00e−58 |
| | | | | 1C7C_A | Chain A, Deoxy Rhb1.1 (Recombinant Hemoglobin). | 222 | 1.00e−58 |
| | | | | 1NQP_C | Chain C, Crystal Structure Of Human Hemoglobin E | 222 | 1.00e−58 |
| | | | | 1O1N_A | Chain A, Deoxy Hemoglobin | 222 | 2.00e−58 |
| | | | | 1AJ9_A | Chain A, Human Carbonmonoxyhemoglobin | 222 | 2.00e−58 |
| | | | | 1O1L_A | Chain A, Deoxy Hemoglobin | 221 | 3.00e−58 |
| | | | | 1C7B_C | Chain C, Deoxy Rhb1.0 | 221 | 3.00e−58 |
| Q62257 | | | M: (C-D) | CAA48671 | alpha1-antichymotrypsin [Homo sapiens]. | 402 | e−112 |
| | | | U: 4 wks | | | | |
| | | | 6.1 | | | | |
| | | | F: 8 wks | | | | |
| | | | 3.49 | | | | |
| | | | | AAD08810 | alpha-1-antichymotrypsin precursor [Homo sapiens]. | 401 | e−111 |
| | | | | NP_001076 | serine (or cysteine) proteinase inhibitor, clade A, member | 400 | e−111 |
| | | | | | 3 precursor [Homo sapiens]. | | |
| | | | | AAH34554 | Serine (or cysteine) proteinase inhibitor, clade A, | 400 | e−111 |
| | | | | ITHUC | alpha-1-antichymotrypsin precursor - human | 394 | e−109 |
| | | | | AAA51560 | alpha-1-antichymotrypsin precursor | 391 | e−108 |
| | | | | 1QMN_A | Chain A, Alpha1-Antichymotrypsin Serpin In The Delta | 385 | e−106 |
| | | | | | Conformation | | |
| | | | | 2ACH_A | Chain A, Alpha1 Antichymotrypsin | 360 | 3.00e−99 |
| | | | | AAA51543 | alpha-1-antichymotrypsin | 357 | 2.00e−98 |
| | | | | 1313184C | chymotrypsin inhibitor | 356 | 6.00e−98 |
| | | | | 4CAA_A | Chain A, Cleaved Antichymotrypsin T345r | 353 | 3.00e−97 |
| | | | | 1AS4_A | Chain A, Cleaved Antichymotrypsin A349r | 353 | 3.00e−97 |
| | | | | 3CAA_A | Chain A, Cleaved Antichymotrypsin A347r | 353 | 3.00e−97 |
| | | | | AAA51547 | alpha-1-antitrypsin precursor | 274 | 2.00e−73 |
| | | | | AAV38262 | serine (or cysteine) proteinase inhibitor, clade A | 274 | 2.00e−73 |
| | JX0129 | | | AAD08810 | alpha-1-antichymotrypsin precursor [Homo sapiens]. | 401 | e−111 |
| | | | | NP_001076 | serine (or cysteine) proteinase inhibitor, clade A, member | 400 | e−111 |
| | | | | | 3 precursor [Homo sapiens]. | | |
| | | | | AAH34554 | Serine (or cysteine) proteinase inhibitor, clade A, | 400 | e−111 |
| | | | | ITHUC | alpha-1-antichymotrypsin precursor - human | 394 | e−109 |
| | | | | AAA51560 | alpha-1-antichymotrypsin precursor | 391 | e−108 |
| | | | | 1QMN_A | Chain A, Alpha1-Antichymotrypsin Serpin In The Delta | 385 | e−106 |
| | | | | | Conformation | | |
| | | | | 2ACH_A | Chain A, Alpha1 Antichymotrypsin | 360 | 3.00e−99 |
| | | | | AAA51543 | alpha-1-antichymotrypsin | 357 | 2.00e−98 |
| | | | | 1313184C | chymotrypsin inhibitor | 356 | 6.00e−98 |
| | | | | 4CAA_A | Chain A, Cleaved Antichymotrypsin T345r | 353 | 3.00e−97 |
| | | | | 1AS4_A | Chain A, Cleaved Antichymotrypsin A349r | 353 | 3.00e−97 |
| | | | | 3CAA_A | Chain A, Cleaved Antichymotrypsin A347r | 353 | 3.00e−97 |
| | | | | AAA51547 | alpha-1-antitrypsin precursor | 274 | 2.00e−73 |
| | | | | AAV38262 | serine (or cysteine) proteinase inhibitor, clade A | 274 | 2.00e−73 |
| | JX0129 | | | AAD08810 | alpha-1-antichymotrypsin precursor [Homo sapiens]. | 401 | e−111 |
| | | | | NP_001076 | serine (or cysteine) proteinase inhibitor, clade A, member | 400 | e−111 |
| | | | | | 3 precursor [Homo sapiens]. | | |
| | | | | AAH34554 | Serine (or cysteine) proteinase inhibitor, clade A, | 400 | e−111 |
| | | | | ITHUC | alpha-1-antichymotrypsin precursor - human | 394 | e−109 |
| | | | | AAA51560 | alpha-1-antichymotrypsin precursor | 391 | e−108 |
| | | | | 1QMN_A | Chain A, Alpha1-Antichymotrypsin Serpin In The Delta | 385 | e−106 |
| | | | | | Conformation | | |
| | | | | 2ACH_A | Chain A, Alpha1 Antichymotrypsin | 360 | 3.00e−99 |
| | | | | AAA51543 | alpha-1-antichymotrypsin | 357 | 2.00e−98 |
| | | | | 1313184C | chymotrypsin inhibitor | 356 | 6.00e−98 |
| | | | | 4CAA_A | Chain A, Cleaved Antichymotrypsin T345r | 353 | 3.00e−97 |
| | | | | 1AS4_A | Chain A, Cleaved Antichymotrypsin A349r | 353 | 3.00e−97 |
| | | | | 3CAA_A | Chain A, Cleaved Antichymotrypsin A347r | 353 | 3.00e−97 |
| | | | | AAA51547 | alpha-1-antitrypsin precursor | 274 | 2.00e−73 |
| | | | | AAV38262 | serine (or cysteine) proteinase inhibitor, clade A | 274 | 2.00e−73 |
| | JX0129 | | | AAD08810 | alpha-1-antichymotrypsin precursor [Homo sapiens]. | 401 | e−111 |
| | | | | NP_001076 | serine (or cysteine) proteinase inhibitor, clade A, member | 400 | e−111 |
| | | | | | 3 precursor [Homo sapiens]. | | |
| | | | | AAH34554 | Serine (or cysteine) proteinase inhibitor, clade A, | 400 | e−111 |
| | | | | ITHUC | alpha-1-antichymotrypsin precursor - human | 394 | e−109 |
| | | | | AAA51560 | alpha-1-antichymotrypsin precursor | 391 | e−108 |
| | | | | 1QMN_A | Chain A, Alpha1-Antichymotrypsin Serpin In The Delta | 385 | e−106 |
| | | | | | Conformation | | |
| | | | | 2ACH_A | Chain A, Alpha1 Antichymotrypsin | 360 | 3.00e−99 |
| | | | | AAA51543 | alpha-1-antichymotrypsin | 357 | 2.00e−98 |
| | | | | 1313184C | chymotrypsin inhibitor | 356 | 6.00e−98 |
| | | | | 4CAA_A | Chain A, Cleaved Antichymotrypsin T345r | 353 | 3.00e−97 |
| | | | | 1AS4_A | Chain A, Cleaved Antichymotrypsin A349r | 353 | 3.00e−97 |
| | | | | 3CAA_A | Chain A, Cleaved Antichymotrypsin A347r | 353 | 3.00e−97 |
| | | | | AAA51547 | alpha-1-antitrypsin precursor | 274 | 2.00e−73 |
| | | | | AAV38262 | serine (or cysteine) proteinase inhibitor, clade A | 274 | 2.00e−73 |
| | JX0129 | | | AAD08810 | alpha-1-antichymotrypsin precursor [Homo sapiens]. | 401 | e−111 |
| | | | | NP_001076 | serine (or cysteine) proteinase inhibitor, clade A, member | 400 | e−111 |
| | | | | | 3 precursor [Homo sapiens]. | | |
| | | | | AAH34554 | Serine (or cysteine) proteinase inhibitor, clade A, | 400 | e−111 |
| | | | | ITHUC | alpha-1-antichymotrypsin precursor - human | 394 | e−109 |
| | | | | AAA51560 | alpha-1-antichymotrypsin precursor | 391 | e−108 |
| | | | | 1QMN_A | Chain A, Alpha1-Antichymotrypsin Serpin In The Delta | 385 | e−106 |
| | | | | | Conformation | | |
| | | | | 2ACH_A | Chain A, Alpha1 Antichymotrypsin | 360 | 3.00e−99 |
| | | | | AAA51543 | alpha-1-antichymotrypsin | 357 | 2.00e−98 |
| | | | | 1313184C | chymotrypsin inhibitor | 356 | 6.00e−98 |
| | | | | 4CAA_A | Chain A, Cleaved Antichymotrypsin T345r | 353 | 3.00e−97 |
| | | | | 1AS4_A | Chain A, Cleaved Antichymotrypsin A349r | 353 | 3.00e−97 |
| | | | | 3CAA_A | Chain A, Cleaved Antichymotrypsin A347r | 353 | 3.00e−97 |
| | | | | AAA51547 | alpha-1-antitrypsin precursor | 274 | 2.00e−73 |
| | | | | AAV38262 | serine (or cysteine) proteinase inhibitor, clade A | 274 | 2.00e−73 |
| I49473 | | | M: (C-D) | AAV38262 | serine (or cysteine) proteinase inhibitor, clade A | 513 | e−145 |
| | | | U: 4 wks | | | | |
| | | | 2.17 | | | | |
| | | | F: 8 wks | | | | |
| | | | 3.19 | | | | |
| | | | | NP_001002236 | serine (or cysteine) proteinase inhibitor, clade A | 512 | e−145 |
| | | | | AAA51547 | alpha-1-antitrypsin precursor | 511 | e−145 |
| | | | | 1012287A | antitrypsin alpha1 mutant | 511 | e−144 |
| | | | | AAH11991 | Serine (or cysteine) proteinase inhibitor, clade A | 511 | e−144 |
| | | | | AAB59495 | alpha-1-antitrypsin | 509 | e−144 |
| | | | | AAF29581 | PRO0684 [Homo sapiens]. | 509 | e−144 |
| | | | | AAA51546 | alpha-1-antitrypsin | 506 | e−143 |
| | | | | 1KCT | Alpha1-Antitrypsin | 498 | e−141 |
| | | | | 1HP7_A | Alpha-1-Antitrypsin | 498 | e−140 |
| | | | | 1QLP_A | Intact Alpha-1-Antitrypsin | 497 | e−140 |
| | | | | 1313184B | alpha1 antitrypsin | 496 | e−140 |
| | | | | 1PSI | Intact Recombined Alpha1-Antitrypsin Mutant Phe 51 | 495 | e−140 |
| | | | | | To Leu. | | |
| | | | | 1IZ2_A | Serpin Protein | 493 | e−139 |
| 556 | | | | AAV38262 | serine (or cysteine) proteinase inhibitor, clade A | 513 | e−145 |
| | | | | NP_001002236 | serine (or cysteine) proteinase inhibitor, clade A | 512 | e−145 |
| | | | | AAA51547 | alpha-1-antitrypsin precursor | 511 | e−145 |
| | | | | 1012287A | antitrypsin alpha1 mutant | 511 | e−144 |
| | | | | AAH11991 | Serine (or cysteine) proteinase inhibitor, clade A | 511 | e−144 |
| | | | | AAB59495 | alpha-1-antitrypsin | 509 | e−144 |
| | | | | AAF29581 | PRO0684 [Homo sapiens]. | 509 | e−144 |
| | | | | AAA51546 | alpha-1-antitrypsin | 506 | e−143 |
| | | | | 1KCT | Alpha1-Antitrypsin | 498 | e−141 |
| | | | | 1HP7_A | Alpha-1-Antitrypsin | 498 | e−140 |
| | | | | 1QLP_A | Intact Alpha-1-Antitrypsin | 497 | e−140 |
| | | | | 1313184B | alpha1 antitrypsin | 496 | e−140 |
| | | | | 1PSI | Intact Recombined Alpha-1-Antitrypsin Mutant Phe 51 | 495 | e−140 |
| | | | | | To Leu. | | |
| | | | | 1IZ2_A | Serpin Protein | 493 | e−139 |
| | I49473 | | | AAV38262 | serine (or cysteine) proteinase inhibitor, clade A | 513 | e−145 |
| | | | | NP_001002236 | serine (or cysteine) proteinase inhibitor, clade A | 512 | e−145 |
| | | | | AAA51547 | alpha-1-antitrypsin precursor | 511 | e−145 |
| | | | | 1012287A | antitrypsin alpha1 mutant | 511 | e−144 |
| | | | | AAH11991 | Serine (or cysteine) proteinase inhibitor, clade A | 511 | e−144 |
| | | | | AAB59495 | alpha-1-antitrypsin | 509 | e−144 |
| | | | | AAF29581 | PRO0684 [Homo sapiens]. | 509 | e−144 |
| | | | | AAA51546 | alpha-1-antitrypsin | 506 | e−143 |
| | | | | 1KCT | Alpha1-Antitrypsin | 498 | e−141 |
| | | | | 1HP7_A | Alpha-1-Antitrypsin | 498 | e−140 |
| | | | | 1QLP_A | Intact Alpha-1-Antitrypsin | 497 | e−140 |
| | | | | 1313184B | alpha1 antitrypsin | 496 | e−140 |
| | | | | 1PSI | Intact Recombined Alpha1-Antitrypsin Mutant Phe 51 | 495 | e−140 |
| | | | | | To Leu. | | |
| | | | | 1IZ2_A | Serpin Protein | 493 | e−139 |
| | I49470 | | | AAV38262 | serine (or cysteine) proteinase inhibitor, clade A | 513 | e−145 |
| | | | | NP_001002236 | serine (or cysteine) proteinase inhibitor, clade A | 512 | e−145 |
| | | | | AAA51547 | alpha-1-antitrypsin precursor | 511 | e−145 |
| | | | | 1012287A | antitrypsin alpha1 mutant | 511 | e−144 |
| | | | | AAH11991 | Serine (or cysteine) proteinase inhibitor, clade A | 511 | e−144 |
| | | | | AAB59495 | alpha-1-antitrypsin | 509 | e−144 |
| | | | | AAF29581 | PRO0684 [Homo sapiens]. | 509 | e−144 |
| | | | | AAA51546 | alpha-1-antitrypsin | 506 | e−143 |
| | | | | 1KCT | Alpha1-Antitrypsin | 498 | e−141 |
| | | | | 1HP7_A | Alpha-1-Antitrypsin | 498 | e−140 |
| | | | | 1QLP_A | Intact Alpha-1-Antitrypsin | 497 | e−140 |
| | | | | 1313184B | alpha1 antitrypsin | 496 | e−140 |
| | | | | 1PSI | Intact Recombined Alpha1-Antitrypsin Mutant Phe 51 | 495 | e−140 |
| | | | | | To Leu. | | |
| | | | | 1IZ2_A | Serpin Protein | 493 | e−139 |
| | I49470 | | | AAV38262 | serine (or cysteine) proteinase inhibitor, clade A | 513 | e−145 |
| | | | | NP_001002236 | serine (or cysteine) proteinase inhibitor, clade A | 512 | e−145 |
| | | | | AAA51547 | alpha-1-antitrypsin precursor | 511 | e−145 |
| | | | | 1012287A | antitrypsin alpha1 mutant | 511 | e−144 |
| | | | | AAH11991 | Serine (or cysteine) proteinase inhibitor, clade A | 511 | e−144 |
| | | | | AAB59495 | alpha-1-antitrypsin | 509 | e−144 |
| | | | | AAF29581 | PRO0684 [Homo sapiens]. | 509 | e−144 |
| | | | | AAA51546 | alpha-1-antitrypsin | 506 | e−143 |
| | | | | 1KCT | Alpha1-Antitrypsin | 498 | e−141 |
| | | | | 1HP7_A | Alpha-1-Antitrypsin | 498 | e−140 |
| | | | | 1QLP_A | Intact Alpha-1-Antitrypsin | 497 | e−140 |
| | | | | 1313184B | alpha1 antitrypsin | 496 | e−140 |
| | | | | 1PSI | Intact Recombined Alpha1-Antitrypsin Mutant Phe 51 | 495 | e−140 |
| | | | | | To Leu. | | |
| | | | | 1IZ2_A | Serpin Protein | 493 | e−139 |
| | I49470 | | | | | | |
| | | | | AAV38262 | serine (or cysteine) proteinase inhibitor, clade A | 513 | e−145 |
| | | | | NP_001002236 | serine (or cysteine) proteinase inhibitor, clade A | 512 | e−145 |
| | | | | AAA51547 | alpha-1-antitrypsin precursor | 511 | e−145 |
| | | | | 1012287A | antitrypsin alpha1 mutant | 511 | e−144 |
| | | | | AAH11991 | Serine (or cysteine) proteinase inhibitor, clade A | 511 | e−144 |
| | | | | AAB59495 | alpha-1-antitrypsin | 509 | e−144 |
| | | | | AAF29581 | PRO0684 [Homo sapiens]. | 509 | e−144 |
| | | | | AAA51546 | alpha-1-antitrypsin | 506 | e−143 |
| | | | | 1KCT | Alpha1-Antitrypsin | 498 | e−141 |
| | | | | 1HP7_A | Alpha-1-Antitrypsin | 498 | e−140 |
| | | | | 1QLP_A | Intact Alpha-1-Antitrypsin | 497 | e−140 |
| | | | | 1313184B | alpha1 antitrypsin | 496 | e−140 |
| | | | | 1PSI | Intact Recombined Alpha1-Antitrypsin Mutant Phe 51 | 495 | e−140 |
| | | | | | To Leu. | | |
| | | | | 1IZ2_A | Serpin Protein | 493 | e−139 |
| I49452 | | | M(C-D) | AAA51547 | alpha-1-antitrypsin precursor | 501 | e−141 |
| | | | U: 4 wks | | | | |
| | | | 2.17 | | | | |
| | | | F: 8 wks | | | | |
| | | | 3.19 | | | | |
| | | | | AAV38262 | serine (or cysteine) proteinase inhibitor, clade A | 501 | e−141 |
| | | | | NP_001002236 | serine (or cysteine) proteinase inhibitor, clade A | 499 | e−141 |
| | | | | 1012287A | antitrypsin alpha1 mutant | 498 | e−141 |
| | | | | AAH11991 | Serine (or cysteine) proteinase inhibitor, clade A | 498 | e−141 |
| | | | | AAB59495 | alpha-1-antitrypsin | 497 | e−140 |
| | | | | AAF29581 | PRO0684 [Homo sapiens]. | 496 | e−140 |
| | | | | AAA51546 | alpha-1-antitrypsin | 493 | e−139 |
| | | | | 1KCT | Alpha1-Antitrypsin | 486 | e−137 |
| | | | | 1HP7_A | Alpha1-Antitrypsin | 486 | e−137 |
| | | Q00897 | | AAV38262 | serine (or cysteine) proteinase inhibitor, clade A | 508 | e−144 |
| | | | | AAA51547 | alpha-1-antitrypsin precursor | 508 | e−144 |
| | | | | NP_001002236 | serine (or cysteine) proteinase inhibitor, clade A | 507 | e−143 |
| | | | | 1012287A | antitrypsin alpha1 mutant | 507 | e−143 |
| | | | | AAH11991 | Serine (or cysteine) proteinase inhibitor, clade A | 506 | e−143 |
| | | | | AAB59495 | alpha-1-antitrypsin | 504 | e−142 |
| | | | | AAF29581 | PRO0684 [Homo sapiens]. | 504 | e−142 |
| | | | | AAA51546 | alpha-1-antitrypsin | 501 | e−141 |
| | | | | 1HP7_A | Alpha1-Antitrypsin | 499 | e−141 |
| | | | | 1KCT | Alpha1-Antitrypsin | 498 | e−141 |
| | I49474 | | | AAV38262 | serine (or cysteine) proteinase inhibitor, clade A | 508 | e−144 |
| | | | | NP_001002236 | serine (or cysteine) proteinase inhibitor, clade A | 507 | e−143 |
| | | | | AAA51547 | alpha-1-antitrypsin precursor | 507 | e−143 |
| | | | | 1012287A | antitrypsin alpha1 mutant | 506 | e−143 |
| | | | | AAH11991 | Serine (or cysteine) proteinase inhibitor, clade A | 506 | e−143 |
| | | | | AAB59495 | alpha-1-antitrypsin | 504 | e−143 |
| | | | | AAF29581 | PRO0684 [Homo sapiens]. | 504 | e−143 |
| | | | | AAA51546 | alpha-1-antitrypsin | 501 | e−142 |
| | | | | 1KCT | Alpha1-Antitrypsin | 494 | e−139 |
| | | | | 1HP7_A | Alpha1-Antitrypsin | 493 | e−139 |
| | I49470 | | | AAV38262 | serine (or cysteine) proteinase inhibitor, clade A | 520 | e−147 |
| | | | | NP_001002236 | serine (or cysteine) proteinase inhibitor, clade A | 518 | e−147 |
| | | | | 1012287A | antitrypsin alpha1 mutant | 518 | e−147 |
| | | | | AAH11991 | Serine (or cysteine) proteinase inhibitor, clade A | 518 | e−147 |
| | | | | AAB59495 | alpha-1-antitrypsin | 517 | e−146 |
| | | | | AAF29581 | PRO0684 [Homo sapiens]. | 516 | e−146 |
| | | | | AAA51546 | alpha-1-antitrypsin | 516 | e−146 |
| | | | | 1KCT | Alpha1-Antitrypsin | 512 | e−145 |
| | | | | 1HP7_A | Alpha1-Antitrypsin | 506 | e−143 |
| | I49473 | | | AAV38262 | serine (or cysteine) proteinase inhibitor, clade A | 513 | e−145 |
| | | | | NP_001002236 | serine (or cysteine) proteinase inhibitor, clade A | 512 | e−145 |
| | | | | AAA51547 | alpha-1-antitrypsin precursor | 511 | e−145 |
| | | | | 1012287A | antitrypsin alpha1 mutant | 511 | e−144 |
| | | | | AAH11991 | Serine (or cysteine) proteinase inhibitor, clade A | 511 | e−144 |
| | | | | AAB59495 | alpha-1-antitrypsin | 509 | e−144 |
| | | | | AAF29581 | PRO0684 [Homo sapiens]. | 509 | e−144 |
| | | | | AAA51546 | alpha-1-antitrypsin | 506 | e−143 |
| | | | | 1KCT | Alpha1-Antitrypsin | 498 | e−141 |
| | | | | 1HP7_A | Alpha1-Antitrypsin | 498 | e−140 |
| Q8C7C7 | | | M(C-D) | AAN17825 | serum albumin [Homo sapiens]. | 828 | 0 |
| | | | F: 4 wks | | | | |
| | | | 2.81 | | | | |
| | | | U: 8 wks | | | | |
| | | | 5.37 | | | | |
| | | | | NP_000468 | albumin precursor [Homo sapiens]. | 828 | 0 |
| | | | | CAA23754 | serum albumin [Homo sapiens]. | 827 | 0 |
| | | | | CAA23753 | unnamed protein product [Homo sapiens]. | 826 | 0 |
| | | | | AAF01333 | serum albumin precursor [Homo sapiens]. | 825 | 0 |
| | | | | CAH18185 | hypothetical protein [Homo sapiens]. | 823 | 0 |
| | | | | AAA98798 | alloalbumin Venezia. | 820 | 0 |
| | | | | 1HA2_A | Chain A, Human Serum Albumin Complexed With | 820 | 0 |
| | | | | | Myristic Acid | | |
| | | | | 1BKE | Human Serum Albumin In A Complex With | 820 | 0 |
| | | | | | Myristic Acid | | |
| | | | | AAH39235 | ALB protein [Homo sapiens]. | 819 | 0 |
| | | | | 1HK5_A | Chain A, Human Serum Albumin Mutant | 818 | 0 |
| | | | | 1HK3_A | Chain A, Human Serum Albumin Mutant | 817 | 0 |
| | | | | 1TF0_A | Chain A, Human Serum Albumin | 814 | 0 |
| | | | | AAG35503 | PRO2619 [Homo sapiens]. | 706 | 0 |
| | | AJ011413 | | NP_000468 | albumin precursor [Homo sapiens]. | 957 | 0 |
| | | | | AAN17825 | serum albumin [Homo sapiens]. | 956 | 0 |
| | | | | CAA23754 | serum albumin [Homo sapiens]. | 955 | 0 |
| | | | | CAA23753 | unnamed protein product [Homo sapiens]. | 953 | 0 |
| | | | | AAH39235 | ALB protein [Homo sapiens]. | 952 | 0 |
| | | | | AAF01333 | serum albumin precursor [Homo sapiens]. | 952 | 0 |
| | | | | AAA98798 | alloalbumin Venezia. | 950 | 0 |
| | | | | CAH18185 | hypothetical protein [Homo sapiens]. | 947 | 0 |
| | | | | 1E7B_A | Chain A, Crystal Structure Of Human Serum Albumin | 920 | 0 |
| | | | | 1HK5_A | Chain A, Human Serum Albumin Mutant | 918 | 0 |
| | | | | 1HK3_A | Chain A, Human Serum Albumin Mutant | 917 | 0 |
| | | | | 1TF0_A | Chain A, Human Serum Albumin | 915 | 0 |
| | | | | 1BKE | Human Serum Albumin In A Complex With Myristic | 915 | 0 |
| | | | | | Acid | | |
| | | | | AAG35503 | PRO2619 [Homo sapiens]. | 780 | 0 |
| | | | | AAA64922 | similar to human albumin | 719 | 0 |
| | Q8C7C7 | | | AAN17825 | serum albumin [Homo sapiens]. | 828 | 0 |
| | | | | NP_000468 | albumin precursor [Homo sapiens]. | 828 | 0 |
| | | | | CAA23754 | serum albumin [Homo sapiens]. | 827 | 0 |
| | | | | CAA23753 | unnamed protein product [Homo sapiens]. | 826 | 0 |
| | | | | AAF01333 | serum albumin precursor [Homo sapiens]. | 825 | 0 |
| | | | | CAH18185 | hypothetical protein [Homo sapiens]. | 823 | 0 |
| | | | | AAA98798 | alloalbumin Venezia. | 820 | 0 |
| | | | | 1HA2_A | Chain A, Human Serum Albumin Complexed With | 820 | 0 |
| | | | | | Myristic Acid | | |
| | | | | 1BKE | Human Serum Albumin In A Complex With Myristic | 820 | 0 |
| | | | | | Acid | | |
| | | | | AAH39235 | ALB protein [Homo sapiens]. | 819 | 0 |
| | | | | 1HK5_A | Chain A, Human Serum Albumin Mutant | 818 | 0 |
| | | | | 1HK3_A | Chain A, Human Serum Albumin Mutant | 817 | 0 |
| | | | | 1TF0_A | Chain A, Human Serum Albumin | 814 | 0 |
| | | | | AAG35503 | PRO2619 [Homo sapiens]. | 706 | 0 |
| | Q8C7C7 | | | AAN17825 | serum albumin [Homo sapiens]. | 828 | 0 |
| | | | | NP_000468 | albumin precursor [Homo sapiens]. | 828 | 0 |
| | | | | CAA23754 | serum albumin [Homo sapiens]. | 827 | 0 |
| | | | | CAA23753 | unnamed protein product [Homo sapiens]. | 826 | 0 |
| | | | | AAF01333 | serum albumin precursor [Homo sapiens]. | 825 | 0 |
| | | | | CAH18185 | hypothetical protein [Homo sapiens]. | 823 | 0 |
| | | | | AAA98798 | alloalbumin Venezia. | 820 | 0 |
| | | | | 1HA2_A | Chain A, Human Serum Albumin Complexed With | 820 | 0 |
| | | | | | Myristic Acid | | |
| | | | | 1BKE | Human Serum Albumin In A Complex With Myristic | 820 | 0 |
| | | | | | Acid | | |
| | | | | AAH39235 | ALB protein [Homo sapiens]. | 819 | 0 |
| | | | | 1HK5_A | Chain A, Human Serum Albumin Mutant | 818 | 0 |
| | | | | 1HK3_A | Chain A, Human Serum Albumin Mutant | 817 | 0 |
| | | | | 1TF0_A | Chain A, Human Serum Albumin | 814 | 0 |
| | | | | AAG35503 | PRO2619 [Homo sapiens]. | 706 | 0 |
| | Q8C7C7 | | | AAN17825 | serum albumin [Homo sapiens]. | 828 | 0 |
| | | | | NP_000468 | albumin precursor [Homo sapiens]. | 828 | 0 |
| | | | | CAA23754 | serum albumin [Homo sapiens]. | 827 | 0 |
| | | | | CAA23753 | unnamed protein product [Homo sapiens]. | 826 | 0 |
| | | | | AAF01333 | serum albumin precursor [Homo sapiens]. | 825 | 0 |
| | | | | CAH18185 | hypothetical protein [Homo sapiens]. | 823 | 0 |
| | | | | AAA98798 | alloalbumin Venezia. | 820 | 0 |
| | | | | 1HA2_A | Chain A, Human Serum Albumin Complexed With | 820 | 0 |
| | | | | | Myristic Acid | | |
| | | | | 1BKE | Human Serum Albumin In A Complex With Myristic | 820 | 0 |
| | | | | | Acid | | |
| | | | | AAH39235 | ALB protein [Homo sapiens]. | 819 | 0 |
| | | | | 1HK5_A | Chain A, Human Serum Albumin Mutant | 818 | 0 |
| | | | | 1HK3_A | Chain A, Human Serum Albumin Mutant | 817 | 0 |
| | | | | 1TF0_A | Chain A, Human Serum Albumin | 814 | 0 |
| | | | | AAG35503 | PRO2619 [Homo sapiens]. | 706 | 0 |
| | Q8C7C7 | | | AAN17825 | serum albumin [Homo sapiens]. | 828 | 0 |
| | | | | NP_000468 | albumin precursor [Homo sapiens]. | 828 | 0 |
| | | | | CAA23754 | serum albumin [Homo sapiens]. | 827 | 0 |
| | | | | CAA23753 | unnamed protein product [Homo sapiens]. | 826 | 0 |
| | | | | AAF01333 | serum albumin precursor [Homo sapiens]. | 825 | 0 |
| | | | | CAH18185 | hypothetical protein [Homo sapiens]. | 823 | 0 |
| | | | | AAA98798 | alloalbumin Venezia. | 820 | 0 |
| | | | | 1HA2_A | Chain A, Human Serum Albumin Complexed With | 820 | 0 |
| | | | | | Myristic Acid | | |
| | | | | 1BKE | Human Serum Albumin In A Complex With Myristic | 820 | 0 |
| | | | | | Acid | | |
| | | | | AAH39235 | ALB protein [Homo sapiens]. | 819 | 0 |
| | | | | 1HK5_A | Chain A, Human Serum Albumin Mutant | 818 | 0 |
| | | | | 1HK3_A | Chain A, Human Serum Albumin Mutant | 817 | 0 |
| | | | | 1TF0_A | Chain A, Human Serum Albumin | 814 | 0 |
| | | | | AAG35503 | PRO2619 [Homo sapiens]. | 706 | 0 |
| AAL34533 | | | M(C-D) | NP_001054 | transferrin [Homo sapiens]. | 1024 | 0 |
| | | | U: 8 wks | | | | |
| | | | 6.6 | | | | |
| | | | F: 4 wks | | | | |
| | | | 2.6 | | | | |
| | | | | AAH59367 | Transferrin [Homo sapiens]. | 1019 | 0 |
| | | | | AAF22007 | PRO1400 [Homo sapiens]. | 851 | 0 |
| | | | | CAA37116 | unnamed protein product [Homo sapiens]. | 801 | 0 |
| | | | | TFHUL | lactotransferrin precursor [validated] - human | 801 | 0 |
| | | | | NP_002334 | lactotransferrin [Homo sapiens]. | 801 | 0 |
| | | | | AAA58656 | HLF2. | 801 | 0 |
| | | | | 1FCK_A | Chain A, Structure Of Diceric Human Lactoferrin | 801 | 0 |
| | | | | 1CB6_A | Chain A, Structure Of Human Apolactoferrin | 801 | 0 |
| | | | | AAN11304 | lactoferrin [Homo sapiens]. | 800 | 0 |
| | | | | AAN75578 | lactoferrin [Homo sapiens]. | 800 | 0 |
| | | | | 1LFH | Lactoferrin | 800 | 0 |
| | | | | 1SQY_A | Chain A, Structure Of Human Diferric Lactoferrin | 800 | 0 |
| | | | | 1LFG | Lactoferrin (Diferric). | 800 | 0 |
| | | | | 1N76_A | Chain A, Crystal Structure Of Human Seminal | 800 | 0 |
| | | | | | Lactoferrin | | |
| | | | | 1LFI | Lactoferrin (Copper Form). | 799 | 0 |
| | | | | 1LCF | Lactoferrin (Copper And Oxalate Form). | 799 | 0 |
| | | | | 1B0L_A | Chain A, Recombinant Human Diferric Lactoferrin | 799 | 0 |
| | | | | AAA59511 | lactoferrin. | 798 | 0 |
| | | | | AAH15822 | Lactotransferrin | 798 | 0 |
| | | | | AAH22347 | Lactotransferrin [Homo sapiens]. | 797 | 0 |
| | | | | AAH15823 | Lactotransferrin [Homo sapiens]. | 795 | 0 |
| | | | | CAA37914 | precursor (AA −19 to 692) [Homo sapiens]. | 794 | 0 |
| | | | | AAA61141 | transferrin. | 752 | 0 |
| | AAL34533 | | | NP_001054 | transferrin [Homo sapiens]. | 1024 | 0 |
| | | | | AAH59367 | Transferrin [Homo sapiens]. | 1019 | 0 |
| | | | | AAF22007 | PRO1400 [Homo sapiens]. | 851 | 0 |
| | | | | CAA37116 | unnamed protein product [Homo sapiens]. | 801 | 0 |
| | | | | TFHUL | lactotransferrin precursor [validated] - human | 801 | 0 |
| | | | | NP_002334 | lactotransferrin [Homo sapiens]. | 801 | 0 |
| | | | | AAA58656 | HLF2. | 801 | 0 |
| | | | | 1FCK_A | Chain A, Structure Of Diceric Human Lactoferrin | 801 | 0 |
| | | | | 1CB6_A | Chain A, Structure Of Human Apolactoferrin | 801 | 0 |
| | | | | AAN11304 | lactoferrin [Homo sapiens]. | 800 | 0 |
| | | | | AAN75578 | lactoferrin [Homo sapiens]. | 800 | 0 |
| | | | | 1LFH | Lactoferrin | 800 | 0 |
| | | | | 1SQY_A | Chain A, Structure Of Human Diferric Lactoferrin | 800 | 0 |
| | | | | 1LFG | Lactoferrin (Diferric). | 800 | 0 |
| | | | | 1N76_A | Chain A, Crystal Structure Of Human Seminal | 800 | 0 |
| | | | | | Lactoferrin | | |
| | | | | 1LFI | Lactoferrin (Copper Form). | 799 | 0 |
| | | | | 1LCF | Lactoferrin (Copper And Oxalate Form). | 799 | 0 |
| | | | | 1B0L_A | Chain A, Recombinant Human Diferric Lactoferrin | 799 | 0 |
| | | | | AAA59511 | lactoferrin. | 798 | 0 |
| | | | | AAH15822 | Lactotransferrin | 798 | 0 |
| | | | | AAH22347 | Lactotransferrin [Homo sapiens]. | 797 | 0 |
| | | | | AAH15823 | Lactotransferrin [Homo sapiens]. | 795 | 0 |
| | | | | CAA37914 | precursor (AA −19 to 692) [Homo sapiens]. | 794 | 0 |
| | | | | AAA61141 | transferrin. | 752 | 0 |
| | AAL34533 | | | NP_001054 | transferrin [Homo sapiens]. | 1024 | 0 |
| | | | | AAH59367 | Transferrin [Homo sapiens]. | 1019 | 0 |
| | | | | AAF22007 | PRO1400 [Homo sapiens]. | 851 | 0 |
| | | | | CAA37116 | unnamed protein product [Homo sapiens]. | 801 | 0 |
| | | | | TFHUL | lactotransferrin precursor [validated] - human | 801 | 0 |
| | | | | NP_002334 | lactotransferrin [Homo sapiens]. | 801 | 0 |
| | | | | AAA58656 | HLF2. | 801 | 0 |
| | | | | 1FCK_A | Chain A, Structure Of Diceric Human Lactoferrin | 801 | 0 |
| | | | | 1CB6_A | Chain A, Structure Of Human Apolactoferrin | 801 | 0 |
| | | | | AAN11304 | lactoferrin [Homo sapiens]. | 800 | 0 |
| | | | | AAN75578 | lactoferrin [Homo sapiens]. | 800 | 0 |
| | | | | 1LFH | Lactoferrin | 800 | 0 |
| | | | | 1SQY_A | Chain A, Structure Of Human Diferric Lactoferrin | 800 | 0 |
| | | | | 1LFG | Lactoferrin (Diferric). | 800 | 0 |
| | | | | 1N76_A | Chain A, Crystal Structure Of Human Seminal | 800 | 0 |
| | | | | | Lactoferrin | | |
| | | | | 1LFI | Lactoferrin (Copper Form). | 799 | 0 |
| | | | | 1LCF | Lactoferrin (Copper And Oxalate Form). | 799 | 0 |
| | | | | 1B0L_A | Chain A, Recombinant Human Diferric Lactoferrin | 799 | 0 |
| | | | | AAA59511 | lactoferrin. | 798 | 0 |
| | | | | AAH15822 | Lactotransferrin | 798 | 0 |
| | | | | AAH22347 | Lactotransferrin [Homo sapiens]. | 797 | 0 |
| | | | | AAH15823 | Lactotransferrin [Homo sapiens]. | 795 | 0 |
| | | | | CAA37914 | precursor (AA −19 to 692) [Homo sapiens]. | 794 | 0 |
| | | | | AAA61141 | transferrin. | 752 | 0 |
| | | AAL34533 | | AAB22049 | transferrin [Homo sapiens]. | 330 | 3.00e−90 |
| | | | | AAH59367 | Transferrin [Homo sapiens]. | 330 | 3.00e−90 |
| | | | | AAF22007 | PRO1400 [Homo sapiens]. | 330 | 3.00e−90 |
| | | | | AAA61141 | transferrin. | 306 | 2.00e−83 |
| | | | | AAH15822 | Lactotransferrin | 241 | 9.00e−64 |

MASTER TABLE 101
Mascot MS Search

| | Appar | | | | | | Calculated | | PBM | E- | Mtch | % |
|---|---|---|---|---|---|---|---|---|---|---|---|---|
| Well# | MW | pI | S# | Behavior | Access# | Mouse Protein Name | MW | pI | Score | value | Pks | Cov |
| A02 | 50 | 6 | 3 | U: (C-D) | Q91XF8 | Apoa4 protein | 45001 | 5.34 | 123 | 8.50e−07 | 12:35 | 26% |
| | | | | +6.3 | | | | | | | | |
| | | | | | B40892 | apolipoprotein A-IV precursor | 44545 | 5.48 | 122 | 1.00e−06 | 12:35 | 26% |
| | | | | | A40892 | apolipoprotein A-IV precursor - mouse | 45001 | 5.41 | 121 | 1.30e−06 | 12:35 | 26% |
| | | | | | Q810I7 | Similar to apolipoprotein A-IV. - Mus musculus | 42889 | 5.55 | 109 | 2.10e−05 | 11:35 | 24% |
| | | | | | Q9DBN0 | Mus musculus adult male liver cDNA | 45016 | 5.34 | 108 | 2.60e−05 | 11:35 | 24% |
| | | | | | | apolipoprotein A-IV | | | | | | |
| | | | | | C40892 | apolipoprotein A-IV precursor - mouse | 45455 | 5.77 | 89 | 0.002 | 10:35 | 21% |
| | | | | | Q01488 | Apolipoprotein A-IV precursor. - | 49223 | 5.86 | 86 | 0.0045 | 10:35 | 19% |
| | | | | | | Mus musculus castaneus | | | | | | |
| | | | | | A25281 | apolipoprotein A-IV precursor - mouse | 44686 | 5.57 | 79 | 0.019 | 9:35 | 21% |
| C02 | 50 | 6 | 4 | U: (C-D) | Q810I7 | Similar to apolipoprotein A-IV. - Mus musculus | 42889 | 5.55 | 41 | 1.40e+02 | 6:49 | 17 |
| | | | | +11.5 | | | | | | | | |
| | | | | | B40892 | apolipoprotein A-IV precursor | 44545 | 5.48 | 29 | 1.90e+03 | 5:49 | 12 |
| | | | | | Q9DBN0 | Mus musculus adult male liver cDNA | 45016 | 5.34 | 29 | 1.90e+03 | 5:49 | 11 |
| | | | | | | apolipoprotein A-IV | | | | | | |
| | | | | | Q91XF8 | Apoa4 protein | 45001 | 5.34 | 29 | 1.90e+03 | 5:49 | 11 |
| | | | | | C40892 | apolipoprotein A-IV precursor - mouse | 45455 | 5.77 | 29 | 2.00e+03 | 5:49 | 11 |
| E02 | 40 | 6.5 | 5 | M: (C-D) | S27001 | alpha-2-macroglobulin - mouse | 165723 | 6.27 | 20 | 3.6 | | |
| C05 | 40 | 6.5 | 8 | M: (C-D) | S27001 | alpha-2-macroglobulin - mouse | 165723 | 6.27 | 20 | 3.6 | | |
| G05 | 40 | 6.5 | 10 | M: (C-D) | S27001 | alpha-2-macroglobulin - mouse | 165723 | 6.27 | 20 | 3.6 | | |
| E08 | 30 | 6.3 | 13 | F: (C-D) | Q8BPD5 | RIKEN apolipoprotei | 30597 | 5.51 | 174 | 6.50e−12 | 14:27 | 40 |
| | | | | −21.9 | | | | | | | | |
| | | | | | JC1237 | apolipoprotein A-I precursor - mouse | 30358 | 5.52 | 158 | 2.60e−10 | 13:27 | 37 |
| | | | | | S22420 | apolipoprotein A-I precursor - mouse | 30569 | 5.64 | 156 | 4.10e−10 | 13:27 | 37 |
| | | | | | O08855 | Apolipoprotein A-I. - Mus musculus (Mouse). | 30526 | 5.51 | 99 | 0.0002 | 15:27 | 40 |
| | | | | | O09042 | Apolipoprotein A-I. - Mus musculus (Mouse). | 30498 | 5.64 | 87 | 0.0036 | 14:27 | 36 |
| G08 | 30 | 6.4 | 14 | F: (C-D) | JC1237 | apolipoprotein A-I precursor - mouse | 30358 | 5.52 | 148 | 2.60e−09 | 13:31 | 37 |
| | | | | −16.1 | | | | | | | | |
| | | | | | Q8BPD5 | RIKEN apolipoprotei | 30597 | 5.51 | 148 | 2.60e−09 | 13:31 | 37 |
| | | | | | S22420 | apolipoprotein A-I precursor - mouse | 30569 | 5.64 | 146 | 4.10e−09 | 13:31 | 37 |
| | | | | | O08855 | Apolipoprotein A-I. - Mus musculus (Mouse). | 30526 | 5.51 | 147 | 3.30e−09 | 13:31 | 36 |
| | | | | | O09042 | Apolipoprotein A-I. - Mus musculus (Mouse). | 30498 | 5.64 | 146 | 4.10e−09 | 13:31 | 36 |
| A11 | 15 | 7 | 15 | M: (C-D) | Q9CY54 | Mus musculus RIKEN full-length hemoglobin | 15768 | 7.14 | 130 | 1.60e−07 | 9:31 | 60 |
| | | | | | | clone: 2500004H04 | | | | | | |
| | | | | | Q91V86 | Mus musculus RIKEN full-length hemoglobin | 15738 | 7.14 | 130 | 1.60e−07 | 9:31 | 60 |
| | | | | | | clone: 2700082N11 | | | | | | |
| | | | | | Q9CY12 | Mus musculus RIKEN full-length hemoglobin | 15753 | 7.14 | 113 | 8.20e−06 | 8:31 | 55 |
| | | | | | | clone: 2510039D09 | | | | | | |
| | | | | | Q9CXH5 | Mus musculus RIKEN full-length hemoglobin | 15681 | 7.14 | 113 | 8.20e−06 | 8:31 | 54 |
| | | | | | | clone: 3300001P16 | | | | | | |
| | | | | | Q9CRZ2 | Mus musculus RIKEN full-length hemoglobin | 14385 | 8.09 | 99 | 0.00022 | 7:31 | 54 |
| | | | | | | clone: 2510040N07 | | | | | | |
| | | | | | BAA77355 | AB020015 NID: - Mus musculus | 15699 | 7.26 | 78 | 0.026 | 6:31 | 38 |
| | | | | | HBMS | hemoglobin beta major chain - mouse | 15830 | 7.12 | 78 | 0.027 | 6:31 | 38 |
| A14 | 45 | 5.4 | 19 | M: (C-D) | Q62257 | Contraspin precursor. - Mus musculus (Mouse). | 46643 | 5.04 | 43 | 84 | 11:36 | 22 |
| C14 | 45 | 5.8 | 20 | M: (C-D) | I49473 | alpha-1 proteinase inhibitor 4 - mouse | 45969 | 5.24 | 163 | 8.20e−11 | 15:49 | 37 |
| | | | | | Q91WH5 | Serpina1a protein (Fragment). - | 45593 | 5.31 | 150 | 1.60e−09 | 14:49 | 38 |
| | | | | | | Mus musculus (Mouse). | | | | | | |
| | | | | | I49470 | alpha-1 proteinase inhibitor 1 - mouse | 45974 | 5.44 | 149 | 2.10e−09 | 14:49 | 37 |
| | | | | | Q91XB8 | Serine (Or cysteine) proteinase inhibitor, | 45937 | 5.32 | 149 | 2.10e−09 | 14:49 | 37 |
| | | | | | | Mus musculus | | | | | | |
| | | | | | Q91V74 | Serpina1a protein. - Mus musculus (Mouse). | 45923 | 5.32 | 149 | 2.10e−09 | 14:49 | 37 |
| | | | | | Q80YB8 | Serpina1a protein (Fragment). - | 47037 | 5.39 | 148 | 2.60e−09 | 14:49 | 36 |
| | | | | | | Mus musculus (Mouse). | | | | | | |
| | | | | | Q91XC1 | Serpina1a protein (Fragment). - | 47128 | 5.2 | 148 | 2.60e−09 | 14:49 | 36 |
| | | | | | | Mus musculus (Mouse). | | | | | | |
| | | | | | I49472 | alpha-1 proteinase inhibitor 3 - mouse | 45825 | 5.32 | 136 | 4.10e−08 | 13:49 | 35 |
| | | | | | Q8VC41 | Serpina1d protein. - Mus musculus (Mouse | 45966 | 5.18 | 106 | 4.10e−05 | 11 | 30 |
| | | | | | | | | | | | of49 | |
| | | | | | I49452 | alpha-1-antitrypsin precursor - mouse | 45866 | 5.33 | 105 | 5.20e−05 | 11 | 30 |
| | | | | | | | | | | | of49 | |
| | | | | | I49471 | alpha-1 proteinase inhibitor 2 - | 44747 | 5.33 | 94 | 0.00068 | 10:49 | 28 |
| | | | | | | mouse (fragment) | | | | | | |
| E14 | 45 | 5.8 | 21 | M: (C-D) | I49473 | alpha-1 proteinase inhibitor 4 - mouse | 45969 | 5.24 | 163 | 8.20e−11 | 15:49 | 37 |
| | | | | | Q91WH5 | Serpina1a protein (Fragment). - | 45593 | 5.31 | 150 | 1.60e−09 | 14:49 | 38 |
| | | | | | | Mus musculus (Mouse). | | | | | | |
| | | | | | I49470 | alpha-1 proteinase inhibitor 1 - mouse | 45974 | 5.44 | 149 | 2.10e−09 | 14:49 | 37 |
| | | | | | Q91XB8 | Serine (Or cysteine) proteinase inhibitor, | 45937 | 5.32 | 149 | 2.10e−09 | 14:49 | 37 |
| | | | | | | Mus musculus | | | | | | |
| | | | | | Q91V74 | Serpina1a protein. - Mus musculus (Mouse). | 45923 | 5.32 | 149 | 2.10e−09 | 14:49 | 37 |
| | | | | | Q80YB8 | Serpina1a protein (Fragment). - | 47037 | 5.39 | 148 | 2.60e−09 | 14:49 | 36 |
| | | | | | | Mus musculus (Mouse). | | | | | | |
| | | | | | Q91XC1 | Serpina1a protein (Fragment). - | 47128 | 5.2 | 148 | 2.60e−09 | 14:49 | 36 |
| | | | | | | Mus musculus (Mouse). | | | | | | |
| | | | | | I49472 | alpha-1 proteinase inhibitor 3 - mouse | 45825 | 5.32 | 136 | 4.10e−08 | 13:49 | 35 |
| | | | | | Q8VC41 | Serpina1d protein. - Mus musculus (Mouse | 45966 | 5.18 | 106 | 4.10e−05 | 11:49 | 30 |
| | | | | | I49452 | alpha-1-antitrypsin precursor - mouse | 45866 | 5.33 | 105 | 5.20e−05 | 11:49 | 30 |
| | | | | | I49471 | alpha-1 proteinase inhibitor 2 - mouse | 44747 | 5.33 | 94 | 0.00068 | 10:49 | 28 |
| | | | | | | (fragment) | | | | | | |
| A17 | 85 | 7 | 23 | M: (C-D) | Q8C7C7 | Mus musculus albumin 1, full insert sequence | 64961 | 5.49 | 85 | 0.005 | 12:56 | 21 |
| | | | | | | clone: C920028B14 | | | | | | |
| | | | | | Q8C7H3 | Mus musculus albumin 1, full insert sequence | 68678 | 5.75 | 77 | 0.034 | 12:56 | 21 |
| | | | | | | clone: C730030P03 | | | | | | |
| | | | | | BAB26650 | AK010025 NID: - Mus musculus | 68648 | 5.75 | 77 | 0.034 | 12:56 | 21 |
| | | | | | A05139 | serum albumin - mouse (fragment) | 51334 | 5.49 | 50 | 17 | 8:56 | 18 |
| C17 | 75 | 7.8 | 24 | M(C-D) | NP_598738 | transferrin [Mus musculus] BAC39532 | 76674 | 6.94 | 111 | 1.30e−05 | 14:42 | 20 |
| | | | | | AAL34533 | AF440692 NID: - Mus musculus | 76628 | 6.92 | 111 | 1.30e−05 | 14:42 | 20 |
| | | | | | AAH08559 | BC008559 NID: - Mus musculus | 69104 | 6.38 | 107 | 3.30e−05 | 13:42 | 20 |
| E17 | 55 | 5.5 | 1 | U: (C-D) | Q62257 | Contraspin precursor. - Mus musculus (Mouse). | 46643 | 5.04 | 71 | 0.12 | 8:41 | 24 |
| | | | | +3.8 | | | | | | | | |
| | | | | | JX0129 | contrapsin precursor - mouse | 46850 | 5.05 | 49 | 22 | 6:41 | 18 |
| | | | | | Q91W80 | Serine (Or cysteine) proteinase inhibitor | 46870 | 5.05 | 49 | 22 | 6:41 | 18 |
| | | | | | Q91X80 | Serine (Or cysteine) proteinase inhibitor | 46822 | 5.05 | 49 | 22 | 6:41 | 18 |
| | | | | | Q8VCH3 | Serine (Or cysteine) proteinase inhibitor | 46836 | 5.05 | 49 | 22 | 6:41 | 18 |
| A20 | 50 | 5.7 | 3 | U: (C-D) | I49471 | alpha-1 proteinase inhibitor 2 - | 44747 | 5.33 | 90 | 0.0017 | 9:32 | 20 |
| | | | | +8.6 | | mouse (fragment | | | | | | |
| | | | | | Q8VC20 | Serine (Or cysteine) proteinase inhibitor, | 45868 | 5.31 | 76 | 0.039 | 8:32 | 17 |
| | | | | | | clade A, | | | | | | |
| | | | | | I49452 | alpha-1-antitrypsin precursor - mouse | 45886 | 5.33 | 76 | 0.046 | 8:32 | 17 |
| | | | | | Q91X22 | Serpina1b protein. - Mus musculus (Mouse). | 45126 | 4.92 | 62 | 1.2 | 7:32 | 13 |
| C23 | 40 | 6.5 | 8 | F: (C-D) | S27001 | alpha-2-macroglobulin - mouse | 165723 | 6.27 | 20 | 3.6 | | |
| | | | | 2.3 | | | | | | | | |
| G23 | 60 | 6.8 | 10 | F: (C-D) | Q91ZP1 | Fibrinogen B-beta-chain (Fragment). - | 27037 | 8.24 | 26 | 2.6 | 8:44 | 31 |
| | | | | 2.1 | | Mus musculus (Mouse). | | | | | | |
| A03 | 35 | 6.6 | 11 | U: (C-D) | AAA37252 | MUSAPOEE NID: - Mus musculus | 33206 | 5.82 | 111 | 1.30e−05 | 11:28 | 37 |
| | | | | +3.2 | | | | | | | | |
| | | | | | JU0036 | apolipoprotein E precursor - mouse | 35844 | 5.56 | 110 | 1.60e−05 | 11:28 | 34 |
| | | | | | AAH28816 | BC028816 NID: - Mus musculus | 35830 | 5.56 | 110 | 1.60e−05 | 11:28 | 34 |
| C03 | 30 | 6.2 | 12 | F: (C-D) | JC1237 | apolipoprotein A-I precursor - mouse | 30358 | 5.52 | 65 | 0.54 | 11:27 | 37 |
| | | | | 2.5 | | | | | | | | |
| | | | | | Q8BPD5 | Mus musculus clone: 3830418K20 product: | 30597 | 5.51 | 64 | 0.59 | 11:27 | 37 |
| | | | | | | apolipoprotei | | | | | | |
| | | | | | S22420 | apolipoprotein A-I precursor - mouse | 30569 | 5.64 | 63 | 0.8 | 11:27 | 37 |
| | | | | | O08855 | Apolipoprotein A-I. - Mus musculus (Mouse). | 30526 | 5.51 | 54 | 7 | 10:27 | 32 |
| | | | | | O09042 | Apolipoprotein A-I. - Mus musculus (Mouse). | 30498 | 5.64 | 52 | 9.4 | 10:27 | 32 |
| E18 | 35 | 6.8 | 3 | F: (C-D) | JU0036 | apolipoprotein E precursor - mouse | 35844 | 5.56 | 114 | 6.50e−06 | 12:36 | 35 |
| | | | | 2.5 | | | | | | | | |
| | | | | | AAH28816 | BC028816 NID: - Mus musculus (NP_033826 | 35830 | 5.56 | 114 | 6.50e−06 | 12:36 | 35 |
| | | | | | | apolipoprotein E [Mus musculus].) | | | | | | |
| | | | | | AAA37252 | MUSAPOEE NID: - Mus musculus | 33206 | 5.82 | 113 | 8.20e−06 | 12:36 | 38 |
| A21 | 35 | 6.8 | 5 | F: (C-D) | JU0036 | apolipoprotein E precursor - mouse | 35844 | 5.56 | 57 | 3.4 | 8:37 | 20 |
| | | | | 3.3 | | | | | | | | |
| | | | | | AAH28816 | BC028816 NID: - Mus musculus (NP_033826 | 35830 | 5.56 | 57 | 3.4 | 8:37 | 20 |
| | | | | | | apolipoprotein E [Mus musculus].) | | | | | | |
| | | | | | AAA37252 | MUSAPOEE NID: - Mus musculus | 33206 | 5.82 | 55 | 5.3 | 8:37 | 22 |

MASTER TABLE 102
Mascot MS/MS Ion Search

| | | | | PBM | E- |
|---|---|---|---|---|---|
| Well # | Fragment | Access# | Mouse Protein Name | Scor | value |
| A02 | 1282.64 | A25281 | apolipoprotein A-IV precursor - mouse | 13 | 2.1e+02 |
| | | Q9DBN0 | Mus musculus adult male liver cDNA | 11 | 2.1e+02 |
| | | | apolipoprotein A-IV | | |
| | | Q91XF8 | Apoa4 protein | 11 | 2.1e+02 |
| | | Q810I7 | Similar to apolipoprotein A-IV. - Mus musculus | 11 | 2.1e+02 |
| | | Q01488 | Apolipoprotein A-IV precursor. - Mus musculus | 11 | 2.1e+02 |
| | | | castaneus | | |
| | | A40892 | apolipoprotein A-IV precursor - mouse | 11 | 2.1e+02 |
| | | C40892 | apolipoprotein A-IV precursor - mouse | 11 | 2.1e+02 |
| | 1305.69 | Q9DBN0 | Mus musculus adult male liver cDNA | 22 | 47 |
| | | | apolipoprotein A-IV | | |
| | | Q91XF8 | Apoa4 protein | 22 | 47 |
| | | Q810I7 | Similar to apolipoprotein A-IV. - Mus musculus | 22 | 47 |
| | | Q01488 | Apolipoprotein A-IV precursor. - Mus musculus | 22 | 47 |
| | | | castaneus | | |
| | | A40892 | apolipoprotein A-IV precursor - mouse | 22 | 47 |
| | | C40892 | apolipoprotein A-IV recursor - mouse | 22 | 47 |
| C02 | 1231.72 | | top 10 scores were not mouse proteins; best score | 6 | 2.00e+03 |
| | | | shown | | |
| | 1298.64 | | top 10 scores were not mouse proteins; best score | 17 | 1.30e+02 |
| | | | shown | | |
| | 1305.69 | | top 10 scores were not mouse proteins; best score | 9 | 9.90e+02 |
| | | | shown | | |
| | 1443.80 | | top 10 scores were not mouse proteins; best score | 10 | 7.10e+02 |
| | | | shown | | |
| | 1461.78 | | top 10 scores were not mouse proteins; best score | 11 | 5.10e+02 |
| | | | shown | | |
| | 1566.87 | | top 3 scores were not mouse proteins; best score | 1 | 5.20e+03 |
| | | | shown | | |
| E02 | 1111.59 | S27001 | alpha-2-macroglobulin - mouse | 20 | 3.6 |
| | 1672.89 | S27001 | alpha-2-macroglobulin - mouse | 19 | 79 |
| C05 | 1111.59 | S27001 | alpha-2-macroglobulin - mouse | 20 | 3.6 |
| | 1672.89 | S27001 | alpha-2-macroglobulin - mouse | 19 | 79 |
| G05 | 1111.59 | S27001 | alpha-2-macroglobulin - mouse | 20 | 3.6 |
| | 1672.89 | S27001 | alpha-2-macroglobulin - mouse | 19 | 79 |
| E08 | 1047.55 | Q8BPD5 | RIKEN apolipoprotei | 16 | 2.2e+02 |
| | | JC1237 | apolipoprotein A-I precursor - mouse | 16 | 2.2e+02 |
| | | O08855 | Apolipoprotein A-I. - Mus musculus (Mouse). | 16 | 2.2e+02 |
| | | O09042 | Apolipoprotein A-I. - Mus musculus (Mouse). | 16 | 2.2e+02 |
| | | S22420 | apolipoprotein A-I precursor - mouse | 16 | 2.2e+02 |
| | 1266.63 | Q6LD50 | Apolipoprotein A1 homolog protein (Fragment). - | 48 | 0.13 |
| | | | Mus sp | | |
| | | Q8BPD5 | RIKEN apolipoprotei | 48 | 0.13 |
| | | JC1237 | apolipoprotein A-I precursor - mouse | 48 | 0.13 |
| | | O08855 | Apolipoprotein A-I. - Mus musculus (Mouse). | 48 | 0.13 |
| | | O09042 | Apolipoprotein A-I. - Mus musculus (Mouse). | 48 | 0.13 |
| | | S22420 | apolipoprotein A-I precursor - mouse | 48 | 0.13 |
| | 1331.64 | Q8BPD5 | RIKEN apolipoprotei | 50 | 0.068 |
| | | JC1237 | apolipoprotein A-I precursor - mouse | 50 | 0.068 |
| | | O08855 | Apolipoprotein A-I. - Mus musculus (Mouse). | 50 | 0.068 |
| | | O09042 | Apolipoprotein A-I. - Mus musculus (Mouse). | 50 | 0.068 |
| | | S22420 | apolipoprotein A-I precursor - mouse | 50 | 0.068 |
| | 1340.72 | Q6LD50 | Apolipoprotein A1 homolog protein (Fragment). - | 23 | 31 |
| | | | Mus sp | | |
| | | Q8BPD5 | RIKEN apolipoprotei | 23 | 31 |
| | | JC1237 | apolipoprotein A-I precursor - mouse | 23 | 31 |
| | | S22420 | apolipoprotein A-I precursor - mouse | 23 | 31 |
| G08 | 1047.53 | Q8BPD5 | RIKEN apolipoprotei | 20 | 93 |
| | | JC1237 | apolipoprotein A-I precursor - mouse | 20 | 93 |
| | | O08855 | Apolipoprotein A-I. - Mus musculus (Mouse). | 20 | 93 |
| | | O09042 | Apolipoprotein A-I. - Mus musculus (Mouse). | 20 | 93 |
| | | S22420 | apolipoprotein A-I precursor - mouse | 20 | 93 |
| | 1266.61 | Q6LD50 | Apolipoprotein A1 homolog protein (Fragment). - | 36 | 1.8 |
| | | | Mus sp | | |
| | | Q8BPD5 | RIKEN apolipoprotei | 36 | 1.8 |
| | | JC1237 | apolipoprotein A-I precursor - mouse | 36 | 1.8 |
| | | O08855 | Apolipoprotein A-I. - Mus musculus (Mouse). | 36 | 1.8 |
| | | O09042 | Apolipoprotein A-I. - Mus musculus (Mouse). | 36 | 1.8 |
| | | S22420 | apolipoprotein A-I precursor - mouse | 36 | 1.8 |
| | 1331.62 | Q8BPD5 | RIKEN apolipoprotei | 58 | 0.013 |
| | | JC1237 | apolipoprotein A-I precursor - mouse | 58 | 0.013 |
| | | O08855 | Apolipoprotein A-I. - Mus musculus (Mouse). | 58 | 0.013 |
| | | O09042 | Apolipoprotein A-I. - Mus musculus (Mouse). | 58 | 0.013 |
| | | S22420 | apolipoprotein A-I precursor - mouse | 58 | 0.013 |
| | 1340.70 | Q8BPD5 | RIKEN apolipoprotei | 16 | 69 |
| | | JC1237 | apolipoprotein A-I precursor - mouse | 16 | 69 |
| | | S22420 | apolipoprotein A-I precursor - mouse | 16 | 69 |
| A11 | 1106.68 | Q9CRZ2 | Mus musculus RIKEN full-length hemoglobin | 38 | 1.4 |
| | | | clone: 2510040N07 | | |
| | | Q9CXH5 | Mus musculus RIKEN full-length hemoglobin | 38 | 1.4 |
| | | | clone: 3300001P16 | | |
| | | Q9CY12 | Mus musculus RIKEN full-length hemoglobin | 38 | 1.4 |
| | | | clone: 2510039D09 | | |
| | | Q9CY54 | Mus musculus RIKEN full-length hemoglobin | 38 | 1.4 |
| | | | clone: 2500004H04 | | |
| | | Q91V86 | Mus musculus RIKEN full-length hemoglobin | 38 | 1.4 |
| | | | clone: 2700082N11 | | |
| | 1294.67 | HBMSN1 | hemoglobin beta minor chain - mouse | 14 | 2.6e+02 |
| | | Q9CRZ2 | Mus musculus RIKEN full-length hemoglobin | 14 | 2.6e+02 |
| | | | clone: 2510040N07 | | |
| | | Q9CXH5 | Mus musculus RIKEN full-length hemoglobin | 14 | 2.6e+02 |
| | | | clone: 3300001P16 | | |
| | | Q9CY12 | Mus musculus RIKEN full-length hemoglobin | 14 | 2.6e+02 |
| | | | clone: 2510039D09 | | |
| | | Q9CY54 | Mus musculus RIKEN full-length hemoglobin | 14 | 2.6e+02 |
| | | | clone: 2500004H04 | | |
| | | Q9QUN8 | Beta-2-globin (Fragment). - Mus musculus (Mouse | 14 | 2.6e+02 |
| | | Q9R0S6 | Beta-1-globin (Fragment). - Mus musculus | 14 | 2.6e+02 |
| | | | (Mouse). | | |
| | 1529.77 | HBA_MOUSE | Hemoglobin alpha chain. - Mus musculus (Mouse). | 27 | 13 |
| | | Q8BPF4 | Mus musculus RIKEN full-length hemoglobin | 27 | 13 |
| | | | clone: 2700073B15 | | |
| | | Q9CY06 | Mus musculus RIKEN full-length hemoglobin | 27 | 13 |
| | | | clone: 2510040P05 | | |
| | | Q9CY10 | Mus musculus RIKEN full-length hemoglobin | 27 | 13 |
| | | | clone: 2510040B16 | | |
| | | Q9QWJ3 | Alpha-1-globin (Fragment). - Mus musculus | 27 | 13 |
| | | | (Mouse). | | |
| | | Q91VB8 | Mus musculus RIKEN full-length hemoglobin | 27 | 13 |
| | | | clone: 2500002C06 | | |
| A14 | 1124.88 | Q8VCH3 | Serine (Or cysteine) proteinase inhibitor Mus | 31 | 6 |
| | | | musculus (Mouse). | | |
| | | Q91W80 | Serine (Or cysteine) proteinase inhibitor Mus | 31 | 6 |
| | | | musculus (Mouse). | | |
| | | Q91X80 | Serine (Or cysteine) proteinase inhibitor Mus | 31 | 6 |
| | | | musculus (Mouse). | | |
| | | JX0129 | contrapsin precursor - mouse | 31 | 6 |
| | | CAA25458 | MMCONT01 NID: - Mus musculus | 31 | 6 |
| | | CAA38948 | MMCMC2 NID: - Mus musculus | 31 | 6 |
| | | Q62257 | Contraspin precursor. - Mus musculus (Mouse). | 31 | 6 |
| | 1343.07 | Q8VCH3 | Serine (Or cysteine) proteinase inhibitor Mus | 60 | 0.0078 |
| | | | musculus (Mouse). | | |
| | | Q91W80 | Serine (Or cysteine) proteinase inhibitor Mus | 60 | 0.0078 |
| | | | musculus (Mouse). | | |
| | | JX0129 | contrapsin precursor - mouse | 60 | 0.0078 |
| | | S23675 | contrapsin-related protein MC-7 precursor - mouse | 60 | 0.0078 |
| | | CAA38948 | MMCMC2 NID: - Mus musculus | 60 | 0.0078 |
| | | Q62257 | Contraspin precursor. - Mus musculus (Mouse). | 60 | 0.0078 |
| | 1630.43 | Q6PG99 | Serpina3g protein (Fragment). - Mus musculus | 54 | 0.023 |
| | | | (Mouse). | | |
| | | Q91WP6 | Serine (Or cysteine) proteinase inhibitor Mus | 54 | 0.023 |
| | | | musculus (Mouse). | | |
| | | JH0493 | alpha-1-antichymotrypsin-like protein EB22/3- | 54 | 0.023 |
| | | | mouse (fragment | | |
| | | S23675 | contrapsin-related protein MC-7 precursor - mouse | 54 | 0.023 |
| | | Q62260 | Serine proteinase inhibitor 2.4 (Fragment). - Mus | 54 | 0.023 |
| | | | musculus (Mouse). | | |
| | | Q62257 | Contraspin precursor. - Mus musculus (Mouse). | 54 | 0.023 |
| | | Q8VCH3 | Serine (Or cysteine) proteinase inhibitor Mus | 54 | 0.023 |
| | | | musculus (Mouse). | | |
| | | Q91W80 | Serine (Or cysteine) proteinase inhibitor Mus | 54 | 0.023 |
| | | | musculus (Mouse). | | |
| | | JX0129 | contrapsin precursor - mouse | 54 | 0.023 |
| | 2143.19 | Q8VCH3 | Serine (Or cysteine) proteinase inhibitor Mus | 59 | 0.0054 |
| | | | musculus (Mouse). | | |
| | | Q91W80 | Serine (Or cysteine) proteinase inhibitor Mus | 59 | 0.0054 |
| | | | musculus (Mouse). | | |
| | | Q91X80 | Serine (Or cysteine) proteinase inhibitor Mus | 59 | 0.0054 |
| | | | musculus (Mouse). | | |
| | | JX0129 | contrapsin precursor - mouse | 59 | 0.0054 |
| | | CAA25458 | MMCONT01 NID: - Mus musculus | 59 | 0.0054 |
| | | CAA38948 | MMCMC2 NID: - Mus musculus | 59 | 0.0054 |
| | | Q62257 | Contraspin precursor. - Mus musculus (Mouse). | 59 | 0.0054 |
| C14 | 1036.54 | I49473 | alpha-1 proteinase inhibitor 4 - mouse | 24 | 33 |
| | 1123.58 | A1T6_MOUSE | Alpha-1-antitrypsin 1-6 | 37 | 1.6 |
| | | Q80YB8 | Serpina1a protein (Fragment). - Mus musculus | 37 | 1.6 |
| | | | (Mouse). | | |
| | | Q91V74 | Serpina1a protein. - Mus musculus (Mouse). | 37 | 1.6 |
| | | Q91WH5 | Serpina1a protein (Fragment). - Mus musculus | 37 | 1.6 |
| | | | (Mouse). | | |
| | | Q91XB8 | Serine (Or cysteine) proteinase inhibitor, Mus | 37 | 1.6 |
| | | | musculus | | |
| | | Q91XC1 | Serpina1a protein (Fragment). - Mus musculus | 37 | 1.6 |
| | | | (Mouse). | | |
| | | A25420 | alpha-1-antitrypsin - mouse (fragment) | 37 | 1.6 |
| | | I49452 | alpha-1-antitrypsin precursor - mouse | 37 | 1.6 |
| | | I49470 | alpha-1 proteinase inhibitor 1 - mouse | 37 | 1.6 |
| | | I49472 | alpha-1 proteinase inhibitor 3 - mouse | 37 | 1.6 |
| | 1999.08 | Q80YB8 | Serpina1a protein (Fragment). - Mus musculus | 7 | 9.10e+02 |
| | | | (Mouse). | | |
| | | Q91V74 | Serpina1a protein. - Mus musculus (Mouse). | 7 | 9.10e+02 |
| | | Q91WH5 | Serpina1a protein (Fragment). - Mus musculus | 7 | 9.10e+02 |
| | | | (Mouse). | | |
| | | Q91XB8 | Serine (Or cysteine) proteinase inhibitor, Mus | 7 | 9.10e+02 |
| | | | musculus | | |
| | | Q91XC1 | Serpina1a protein (Fragment). - Mus musculus | 7 | 9.10e+02 |
| | | | (Mouse). | | |
| | | I49470 | alpha-1 proteinase inhibitor 1 - mouse | 7 | 9.10e+02 |
| | | I49472 | alpha-1 proteinase inhibitor 3 - mouse | 7 | 9.10e+02 |
| | 2405.17 | Q8VC20 | Serine (Or cysteine) proteinase inhibitor | 45 | 11 |
| | | Q80YB8 | Serpina1a protein (Fragment). - Mus musculus | 45 | 11 |
| | | | (Mouse). | | |
| | | Q91V74 | Serpina1a protein. - Mus musculus (Mouse). | 45 | 11 |
| | | Q91WH5 | Serpina1a protein (Fragment.). - Mus musculus | 45 | 11 |
| | | | (Mouse). | | |
| | | Q91X22 | Serpina1b protein. - Mus musculus (Mouse). | 45 | 11 |
| | | Q91XC1 | Serpina1a protein (Fragment). - Mus musculus | 45 | 11 |
| | | | (Mouse). | | |
| | | I49452 | alpha-1-antitrypsin precursor - mouse | 45 | 11 |
| | | I49470 | alpha-1 proteinase inhibitor 1 - mouse | 45 | 11 |
| | | I49471 | alpha-1 proteinase inhibitor 2 - mouse (fragment) | 45 | 11 |
| | | I49472 | alpha-1 proteinase inhibitor 3 - mouse | 45 | 11 |
| | | I49474 | alpha-1 proteinase inhibitor 5 - mouse | 45 | 11 |
| | 2549.17 | A1T6_MOUSE | Alpha-1-antitrypsin 1-6 | 36 | 0.74 |
| | | Q80YB8 | Serpina1a protein (Fragment). - Mus musculus | 36 | 0.74 |
| | | | (Mouse). | | |
| | | Q91V74 | Serpina1a protein. - Mus musculus (Mouse). | 36 | 0.74 |
| | | Q91WH5 | Serpina1a protein (Fragment). - Mus musculus | 36 | 0.74 |
| | | | (Mouse). | | |
| | | Q91XB8 | Serine (Or cysteine) proteinase inhibitor, Mus | 36 | 0.74 |
| | | | musculus | | |
| | | Q91XC1 | Serpina1a protein (Fragment). - Mus musculus | 36 | 0.74 |
| | | | (Mouse). | | |
| | | A25420 | alpha-1-antitrypsin - mouse (fragment) | 36 | 0.74 |
| | | I49470 | alpha-1 proteinase inhibitor 1 - mouse | 36 | 0.74 |
| | | I49472 | alpha-1 proteinase inhibitor 3 - mouse | 36 | 0.74 |
| E14 | 1036.54 | I49473 | alpha-1 proteinase inhibitor 4 - mouse | 24 | 33 |
| | 1123.58 | A1T6_MOUSE | Alpha-1-antitrypsin 1-6 | 37 | 1.6 |
| | | Q80YB8 | Serpina1a protein (Fragment). - Mus musculus | 37 | 1.6 |
| | | | (Mouse). | | |
| | | Q91V74 | Serpina1a protein. - Mus musculus (Mouse). | 37 | 1.6 |
| | | Q91WH5 | Serpina1a protein (Fragment). - Mus musculus | 37 | 1.6 |
| | | | (Mouse). | | |
| | | Q91XB8 | Serine (Or cysteine) proteinase inhibitor, Mus | 37 | 1.6 |
| | | | musculus | | |
| | | Q91XC1 | Serpina1a protein (Fragment). - Mus musculus | 37 | 1.6 |
| | | | (Mouse). | | |
| | | A25420 | alpha-1-antitrypsin - mouse (fragment) | 37 | 1.6 |
| | | I49452 | alpha-1-antitrypsin precursor - mouse | 37 | 1.6 |
| | | I49470 | alpha-1 proteinase inhibitor 1 - mouse | 37 | 1.6 |
| | | I49472 | alpha-1 proteinase inhibitor 3 - mouse | 37 | 1.6 |
| | 1999.08 | Q80YB8 | Serpina1a protein (Fragment). - Mus musculus | 7 | 9.10e+02 |
| | | | (Mouse). | | |
| | | Q91V74 | Serpina1a protein. - Mus musculus (Mouse). | 7 | 9.10e+02 |
| | | Q91WH5 | Serpina1a protein (Fragment). - Mus musculus | 7 | 9.10e+02 |
| | | | (Mouse). | | |
| | | Q91XB8 | Serine (Or cysteine) proteinase inhibitor, Mus | 7 | 9.10e+02 |
| | | | musculus | | |
| | | Q91XC1 | Serpina1a protein (Fragment). - Mus musculus | 7 | 9.10e+02 |
| | | | (Mouse). | | |
| | | I49470 | alpha-1 proteinase inhibitor 1 - mouse | 7 | 9.10e+02 |
| | | I49472 | alpha-1 proteinase inhibitor 3 - mouse | 7 | 9.10e+02 |
| | 2405.17 | Q8VC20 | Serine (Or cysteine) proteinase inhibitor | 45 | 11 |
| | | Q80YB8 | Serpina1a protein (Fragment). - Mus musculus | 45 | 11 |
| | | | (Mouse). | | |
| | | Q91V74 | Serpina1a protein. - Mus musculus (Mouse). | 45 | 11 |
| | | Q91WH5 | Serpina1a protein (Fragment). - Mus musculus | 45 | 11 |
| | | | (Mouse). | | |
| | | Q91X22 | Serpina1b protein. - Mus musculus (Mouse). | 45 | 11 |
| | | Q91XC1 | Serpina1a protein (Fragment). - Mus musculus | 45 | 11 |
| | | | (Mouse). | | |
| | | I49452 | alpha-1-antitrypsin precursor - mouse | 45 | 11 |
| | | I49470 | alpha-1 proteinase inhibitor 1 - mouse | 45 | 11 |
| | | I49471 | alpha-1 proteinase inhibitor 2 - mouse (fragment) | 45 | 11 |
| | | I49472 | alpha-1 proteinase inhibitor 3 - mouse | 45 | 11 |
| | | I49474 | alpha-1 proteinase inhibitor 5 - mouse | 45 | 11 |
| | 2549.17 | A1T6_MOUSE | Alpha-1-antitrypsin 1-6 | 36 | 0.74 |
| | | Q80YB8 | Serpina1a protein (Fragment). - Mus musculus | 36 | 0.74 |
| | | | (Mouse). | | |
| | | Q91V4 | Serpina1a protein. - Mus musculus (Mouse). | 36 | 0.74 |
| | | Q91WH5 | Serpina1a protein (Fragment). - Mus musculus | 36 | 0.74 |
| | | | (Mouse). | | |
| | | Q91XB8 | Serine (Or cysteine) proteinase inhibitor, Mus | 36 | 0.74 |
| | | | musculus | | |
| | | Q91XC1 | Serpina1a protein (Fragment). - Mus musculus | 36 | 0.74 |
| | | | (Mouse). | | |
| | | A25420 | alpha-1-antitrypsin - mouse (fragment) | 36 | 0.74 |
| | | I49470 | alpha-1 proteinase inhibitor 1 - mouse | 36 | 0.74 |
| | | I49472 | alpha-1 proteinase inhibitor 3 - mouse | 36 | 0.74 |
| A17 | 1149.61 | Q8C7C7 | Mus musculus albumin 1, full insert sequence | 57 | 0.017 |
| | | | clone: C920028B14 | | |
| | | Q8C7H3 | Mus musculus albumin 1, full insert sequence | 57 | 0.017 |
| | | | clone: C730030P03 | | |
| | | Q8CG74 | Albumin (Fragment). - Mus musculus (Mouse). | 57 | 0.017 |
| | | BAB26650 | AK010025 NID: - Mus musculus | 57 | 0.017 |
| | 1250.58 | Q8CG74 | Albumin (Fragment). - Mus musculus (Mouse). | 54 | 0.03 |
| | | Q8C7C7 | Mus musculus albumin 1, full insert sequence | 54 | 0.03 |
| | | | clone: C920028B14 | | |
| | | Q8C7H3 | Mus musculus albumin 1, full insert sequence | 54 | 0.03 |
| | | | clone: C730030P03 | | |
| | | BAB26650 | AK010025 NID: - Mus musculus | 54 | 0.03 |
| | 1439.79 | Q8C7C7 | Mus musculus albumin 1, full insert sequence | 64 | 0.0028 |
| | | | clone: C920028B14 | | |
| | | Q8C7H3 | Mus musculus albumin 1, full insert sequence | 64 | 0.0028 |
| | | | clone: C730030P03 | | |
| | | A05139 | serum albumin - mouse (fragment | 64 | 0.0028 |
| | | BAB26650 | AK010025 NID: - Mus musculus | 64 | 0.0028 |
| | 1479.80 | A05139 | serum albumin - mouse (fragment | 44 | 0.27 |
| | | AAA37190 | MUSAFPA NID: - Mus musculus | 44 | 0.27 |
| | | Q8C7C7 | Mus musculus albumin 1, full insert sequence | 44 | 0.27 |
| | | | clone: C920028B14 | | |
| | | Q8C7H3 | Mus musculus albumin 1, full insert sequence | 44 | 0.27 |
| | | | clone: C730030P03 | | |
| | | BAB26650 | AK010025 NID: - Mus musculus | 44 | 0.27 |
| C17 | 1306.55 | AAH08559 | BC008559 NID: - Mus musculus | 46 | 0.17 |
| | | A28446 | transferrin - mouse (fragments) | 46 | 0.17 |
| | | AAL34533 | AF440692 NID: - Mus musculus | 46 | 0.17 |
| | | BAC39532 | AK085754 NID: - Mus musculus | 46 | 0.17 |
| | | AAA40491 | MUSTSFA4 NID: - Mus musculus | 46 | 0.17 |
| E17 | 1124.62 | Q8VCH3 | Serine (Or cysteine) proteinase inhibitor | 14 | 2.80e+02 |
| | | Q91W80 | Serine (Or cysteine) proteinase inhibitor | 14 | 2.80e+02 |
| | | Q91X80 | Serine (Or cysteine) proteinase inhibitor | 14 | 2.80e+02 |
| | | JX0129 | contrapsin precursor - mouse | 14 | 2.80e+02 |
| | | Q62257 | Contraspin precursor. - Mus musculus (Mouse). | 14 | 2.80e+02 |
| | 1629.76 | Q6PG99 | Serpina3g protein (Fragment). - Mus musculus | 21 | 47 |
| | | | (Mouse). | | |
| | | Q91WP6 | Serine (Or cysteine) proteinase inhibitor | 21 | 47 |
| | | JH0493 | alpha-1-antichymotrypsin-like protein EB22/3 - | 21 | 47 |
| | | | mouse | | |
| | | JH0494 | alpha-1-antichymotrypsin-like protein EB22/4 - | 21 | 47 |
| | | | mouse | | |
| | | S23675 | contrapsin-related protein MC-7 precursor - mouse | 21 | 47 |
| | | Q62257 | Contraspin precursor. - Mus musculus (Mouse | 21 | 47 |
| | 1737.78 | Q62257 | Contraspin precursor. - Mus musculus (Mouse | 34 | 2.3 |
| A20 | 1241.61 | Q8VC41 | Serpina1d protein. - Mus musculus (Mouse). | 36 | 1.8 |
| | | I49452 | alpha-1-antitrypsin precursor - mouse | 36 | 1.8 |
| | | I49471 | alpha-1 proteinase inhibitor 2 - mouse (fragment | 36 | 1.8 |
| | | I49473 | alpha-1 proteinase inhibitor 4 - mouse | 36 | 1.8 |
| | | I49474 | alpha-1 proteinase inhibitor 5 - mouse | 36 | 1.8 |
| | 2002.98 | A1T6_MOUSE | Alpha-1-antitrypsin 1-6 | 46 | 0.12 |
| | | Q8VC20 | Serine (Or cysteine) proteinase inhibitor, | 46 | 0.12 |
| | | Q8VC41 | Serpina1d protein. - Mus musculus (Mouse | 46 | 0.12 |
| | | Q80YB8 | Serpina1a protein (Fragment). - Mus musculus | 46 | 0.12 |
| | | | (Mouse). | | |
| | | Q91V74 | Serpina1a protein. - Mus musculus (Mouse). | 46 | 0.12 |
| | | Q91WH5 | Serpina1a protein (Fragment). - Mus musculus | 46 | 0.12 |
| | | | (Mouse). | | |
| | | Q91X22 | Serpina1b protein. - Mus musculus (Mouse). | 46 | 0.12 |
| | | Q91XB8 | Serine (Or cysteine) proteinase inhibitor, | 46 | 0.12 |
| | | Q91XC1 | Serpina1a protein (Fragment). - Mus musculus | 46 | 0.12 |
| | | | (Mouse). | | |
| | | A25420 | alpha-1-antitrypsin - mouse (fragment) | 46 | 0.12 |
| | | I49452 | alpha-1-antitrypsin precursor - mouse | 46 | 0.12 |
| | | I49470 | alpha-1 proteinase inhibitor 1 - mouse | 46 | 0.12 |
| | | I49471 | alpha-1 proteinase inhibitor 2 - mouse (fragment) | 46 | 0.12 |
| | | I49472 | alpha-1 proteinase inhibitor 3 - mouse | 46 | 0.12 |
| | | I49473 | alpha-1 proteinase inhibitor 4 - mouse | 46 | 0.12 |
| | 2031.92 | Q8VC20 | Serine (Or cysteine) proteinase inhibitor, | 79 | 5.30e−05 |
| | | I49452 | alpha-1-antitrypsin precursor - mouse | 79 | 5.30e−05 |
| | | I49471 | alpha-1 proteinase inhibitor 2 - mouse (fragment) | 79 | 5.30e−05 |
| C23 | 1111.59 | S27001 | alpha-2-macroglobulin - mouse | 20 | 3.6 |
| | 1672.89 | S27001 | alpha-2-macroglobulin - mouse | 19 | 79 |
| G23 | 1250.46 | Q91ZP1 | fibrinogen b-beta-chain | 56 | 0.013 |
| | | Q8K0E8 | fibrinogen b-beta polypeptide | 56 | 0.013 |
| | 1439.66 | Q91ZP1 | fibrinogen b-beta-chain | 48 | 0.056 |
| | | Q8K0E8 | fibrinogen b-beta polypeptide | 48 | 0.056 |
| A03 | 1599.85 | JU0036 | apolipoprotein E precursor - mouse | 23 | 30 |
| | | AAH28816 | BC028816 NID: - Mus musculus | 23 | 30 |
| | | AAA37252 | MUSAPOEE NID: - Mus musculus | 23 | 30 |
| C03 | 1047.43 | Q8BPD5 | Mus musculus clone: 3830418K20 | 13 | 4.60e+02 |
| | | | product: apolipoprotei | | |
| | | JC1237 | apolipoprotein A-I precursor - mouse | 13 | 4.60e+02 |
| | | O08855 | Apolipoprotein A-I. - Mus musculus (Mouse). | 13 | 4.60e+02 |
| | | O09042 | Apolipoprotein A-I. - Mus musculus (Mouse). | 13 | 4.60e+02 |
| | | S22420 | apolipoprotein A-I precursor - mouse | 13 | 4.60e+02 |
| | 1266.36 | Q8BPD5 | Mus musculus clone: 3830418K20 | 29 | 10 |
| | | | product: apolipoprotei | | |
| | | JC1237 | apolipoprotein A-I precursor - mouse | 29 | 10 |
| | | O08855 | Apolipoprotein A-I. - Mus musculus (Mouse). | 29 | 10 |
| | | O09042 | Apolipoprotein A-I. - Mus musculus (Mouse). | 29 | 10 |
| | | S22420 | apolipoprotein A-I precursor - mouse | 29 | 10 |
| | 1331.32 | Q8BPD5 | Mus musculus clone: 3830418K20 | 36 | 1.9 |
| | | | product: apolipoprotei | | |
| | | JC1237 | apolipoprotein A-I precursor - mouse | 36 | 1.9 |
| | | O08855 | Apolipoprotein A-I. - Mus musculus (Mouse). | 36 | 1.9 |
| | | O09042 | Apolipoprotein A-I. - Mus musculus (Mouse). | 36 | 1.9 |
| | | S22420 | apolipoprotein A-I precursor - mouse | 36 | 1.9 |
| E18 | 1075.53 | JU0036 | apolipoprotein E precursor - mouse | 28 | 15 |
| | | AAH28816 | BC028816 NID: - Mus musculus | 28 | 15 |
| | | AAA37252 | MUSAPOEE NID: - Mus musculus | 28 | 15 |
| | 1239.61 | JU0036 | apolipoprotein E precursor - mouse | 17 | 1.50e+02 |
| | | AAH28816 | BC028816 NID: - Mus musculus | 17 | 1.50e+02 |
| | | AAA37252 | MUSAPOEE NID: - Mus musculus | 17 | 1.50e+02 |
| | 1599.72 | JU0036 | apolipoprotein E precursor - mouse | 61 | 0.0051 |
| | | AAH28816 | BC028816 NID: - Mus musculus | 61 | 0.0051 |
| | | AAA37252 | MUSAPOEE NID: - Mus musculus | 61 | 0.0051 |
| | 1743.74 | JU0036 | apolipoprotein E precursor - mouse | 43 | 0.28 |
| | | AAH28816 | BC028816 NID: - Mus musculus | 43 | 0.28 |
| | | AAA37252 | MUSAPOEE NID: - Mus musculus | 43 | 0.28 |
| A21 | 1162.36 | JU0036 | apolipoprotein E precursor - mouse | 34 | 3.1 |
| | | AAH28816 | BC028816 NID: - Mus musculus | 34 | 3.1 |
| | | AAA37252 | MUSAPOEE NID: - Mus musculus | 34 | 3.1 |
| | 1239.44 | JU0036 | apolipoprotein E precursor - mouse | 21 | 61 |
| | | AAH28816 | BC028816 NID: - Mus musculus | 21 | 61 |
| | | AAA37252 | MUSAPOEE NID: - Mus musculus | 21 | 61 |
| | 1599.29 | JU0036 | apolipoprotein E precursor - mouse | 81 | 4.50e−05 |
| | | AAH28816 | BC028816 NID: - Mus musculus | 81 | 4.50e−05 |
| | | AAA37252 | MUSAPOEE NID: - Mus musculus | 81 | 4.50e−05 |

MASTER TABLE 103
MS-FIT Search

| | Appar | | | | Calculated | | PBM | Mtch | % |
|---|---|---|---|---|---|---|---|---|---|
| Well # | MW | pI | Access# | Mouse Protein Name | Mol Wt | pI | Score | Pks | Cov |
| A02 | 50 | 6 | B40892 | apolipoprotein A-IV precursor | 44572.8 | 5.48 | 3.30e+06 | 17:35 | 48 |
| | | | P06728 | APOLIPOPROTEIN A-IV PRECURSOR | 45029.3 | 5.41 | 3.26e+06 | 17:35 | 48 |
| | | | C40892 | apolipoprotein A-IV precursor | 45483.9 | 5.77 | 2.84e+05 | 15:35 | 42 |
| | | | M64250 | MUSALPAIVC | 49254.1 | 5.86 | 2.62e+05 | 15:35 | 42 |
| | | | M13966 | MUS MUSCULUS. MUSAPOIVA | 44714 | 5.57 | 4.02e+04 | 12:35 | 34 |
| C02 | 50 | 6 | B40892 | apolipoprotein A-IV precursor | 44572.8 | 5.48 | 175 | 8:49 | 16 |
| | | | P06728 | APOLIPOPROTEIN A-IV PRECURSOR | 45029.3 | 5.41 | 174 | 8:49 | 16 |
| | | | C40892 | apolipoprotein A-IV precursor | 45483.9 | 5.77 | 41.9 | 7:49 | 14 |
| | | | M64250 | MUSALPAIVC | 49254.1 | 5.86 | 38.7 | 7:49 | 14 |
| E02 | 40 | 6.5 | | none of top 50 scorers consistent with Mascot results | | | | | |
| C05 | 40 | 6.5 | | none of top 50 scorers consistent with Mascot results | | | | | |
| G05 | 40 | 6.5 | | none of top 50 scorers consistent with Mascot results | | | | | |
| E08 | 30 | 6.3 | Q00623 | MUS MUSCULUS. APOLIPOPROTEIN A-I PRECURSOR | 30587.6 | 5.65 | 5.93e+05 | 14:27 | 51 |
| G08 | 30 | 6.4 | Q00623 | MUS MUSCULUS. APOLIPOPROTEIN A-I PRECURSOR | 30587.6 | 5.65 | 6.20e+05 | 0.8014 | 45 |
| A11 | 15 | 7 | P02088 | HBB1_MOUSE. MUS MUSCULUS. HEMOGLOBIN BETA-1 | 15709 | 7.26 | 5.39e+03 | 7:31 | 22 |
| | | | | CHAIN (B1) (MAJOR | | | | | |
| | | | P02089 | HBB2_MOUSE. MUS MUSCULUS. HEMOGLOBIN BETA-2 | 15747 | 7.97 | 538 | 4:31 | 12 |
| | | | | CHAIN (B2) (MINOR | | | | | |
| A14 | 45 | 5.4 | | none of top 50 scorers consistent with Mascot results | | | | | |
| C14 | 45 | 5.8 | Q00897 | MOUSE ALPHA-1 ANTITRYPSIN 1-4 PRECURSOR | 45998 | 5.24 | 3.39e+08 | 15:49 | 30 |
| | | | | (SERINE PROTEASE INHIBITOR 1-4) . . . | | | | | |
| | | | Q00896 | MOUSE ALPHA-1 ANTITRYPSIN 1-4 PRECURSOR | 45854 | 5.31 | 9.27e+07 | 13:49 | 26 |
| | | | | (SERINE PROTEASE INHIBITOR 1-4) . . . | | | | | |
| | | | P07758 | MOUSE ALPHA-1 ANTITRYPSIN 1-1 PRECURSOR | 46002.8 | 5.44 | 9.24e+07 | 13:49 | 26 |
| | | | P22599 | MOUSE ALPHA-1 ANTITRYPSIN 1-2 PRECURSOR | 54914 | 5.33 | 4.21e+06 | 11:49 | 22 |
| | | | I49471 | MOUSE. alpha-1 proteinase inhibitor 2. | 44775.4 | 5.33 | 1.35e+06 | 10:49 | 20 |
| | | | P81105 | MOUSE ALPHA-1 ANTITRYPSIN 1-6 | 23745 | 5.97 | 2.45e+05 | 6:49 | 12 |
| E14 | 45 | 5.8 | Q00897 | MOUSE ALPHA-1 ANTITRYPSIN 1-4 PRECURSOR | 45998 | 5.24 | 3.39e+08 | 15:49 | 30 |
| | | | | (SERINE PROTEASE INHIBITOR 1-4) . . . | | | | | |
| | | | Q00896 | MOUSE ALPHA-1 ANTITRYPSIN 1-4 PRECURSOR | 45854 | 5.31 | 9.27e+07 | 13:49 | 26 |
| | | | | (SERINE PROTEASE INHIBITOR 1-4) . . . | | | | | |
| | | | P07758 | MOUSE ALPHA-1 ANTITRYPSIN 1-1 PRECURSOR | 46002.8 | 5.44 | 9.24e+07 | 13:49 | 26 |
| | | | P22599 | MOUSE ALPHA-1 ANTITRYPSIN 1-2 PRECURSOR | 54914 | 5.33 | 4.21e+06 | 11:49 | 22 |
| | | | I49471 | MOUSE. alpha-1 proteinase inhibitor 2. | 44775.4 | 5.33 | 1.35e+06 | 10:49 | 20 |
| | | | P81105 | MOUSE ALPHA-1 ANTITRYPSIN 1-6 | 23745 | 5.97 | 2.45e+05 | 6:49 | 12 |
| A17 | 85 | 7 | AJ011413 | HOUSE MOUSE. MMU011413 NID | 68693 | 5.75 | 5.90e+06 | 14:56 | 25 |
| | | | P07724 | MUS MUSCULUS. SERUM ALBUMIN (FRAGMENTS) | 49558 | 5.35 | 8.13e+03 | 8:56 | 14 |
| | | | L31395 | MUS MUSCULUS. MUSBRAD | 85040 | 5.97 | 7.09e+03 | 8:56 | 14 |
| C17 | 75 | 7.8 | | none of top 50 scorers consistent with Mascot results | | | | | |
| E17 | 55 | 5.5 | | none of top 50 scorers consistent with Mascot results | | | | | |
| A20 | 50 | 5.7 | | none of top 50 scorers consistent with Mascot results | | | | | |
| C23 | 40 | 6.5 | | none of top 50 scorers consistent with Mascot results | | | | | |
| G23 | 60 | 6.8 | | none of top 50 scorers consistent with Mascot results | | | | | |
| A03 | 35 | 6.6 | P08226 | MUS MUSCULUS. APOLIPOPROTEIN E PRECURSOR (APO-E).. | 35866 | 5.56 | 6.73e+05 | 12:28 | 42 |
| C03 | 30 | 6.2 | | none of top 50 scorers consistent with Mascot results | | | | | |
| E18 | 35 | 6.8 | | none of top 50 scorers consistent with Mascot results | | | | | |
| A21 | 35 | 6.8 | | none of top 50 scorers consistent with Mascot results | | | | | |

### REFERENCES

### Numbered Citations Only

1. Tisch R, McDevitt H 1996 Insulin-dependent diabetes mellitus. Cell 85:291-7.
2. Zimmet P, Alberti K G, Shaw J 2001 Global and societal implications of the diabetes epidemic. Nature 414:782-7
3. Kahn S E 2001 Clinical review 135: The importance of beta-cell failure in the development and progression of type 2 diabetes. J Clin Endocrinol Metab 86:4047-58
4. Bell G I, Polonsky K S 2001 Diabetes mellitus and genetically programmed defects in beta-cell function. Nature 414:788-91
5. Ramlo-Halsted B A, Edelman S V 1999 The natural history of type 2 diabetes. Implications for clinical practice. Prim Care 26:771-89.
6. Reaven G M 1988 Banting lecture 1988. Role of insulin resistance in human disease. Diabetes 37:1595-607.
7. Wickelgren 11998 Obesity: how big a problem? Science 280:1364-7.
8. Friedman J M, Leibel R L 1992 Tackling a weighty problem. Cell 69:217-20.
9. Kopelman P G 2000 Obesity as a medical problem. Nature 404:635-43.
10. Hogan P, Dall T, Nikolov P 2003 Economic costs of diabetes in the US in 2002. Diabetes Care 26:917-32
11. Bjorntorp P 1991 Metabolic implications of body fat distribution. Diabetes Care 14:1132-43.
12. Emery E M, Schmid T L, Kahn H S, Filozof P P 1993 A review of the association between abdominal fat distribution, health outcome measures, and modifiable risk factors. Am J Health Promot 7:342-53.
13. Clark M G, Rattigan S, Clark D G 1983 Obesity with insulin resistance: experimental insights. Lancet 2:1236-40.
14. Kissebah A H, Vydelingum N, Murray R, et al. 1982 Relation of body fat distribution to metabolic complications of obesity. J Clin Endocrinol Metab 54:254-60.
15. Kissebah A H 1996 Intra-abdominal fat: is it a major factor in developing diabetes and coronary artery disease? Diabetes Res Clin Pract 30 Suppl:25-30.
16. Lewis G F, Carpentier A, Adeli K, Giacca A 2002 Disordered fat storage and mobilization in the pathogenesis of insulin resistance and type 2 diabetes. Endocr Rev 23:201-29.
17. Masuzaki H, Paterson J, Shinyama H, et al. 2001 A transgenic model of visceral obesity and the metabolic syndrome. Science 294:2166-70.
18. Bjorntorp P 1990 “Portal” adipose tissue as a generator of risk factors for cardiovascular disease and diabetes. Arteriosclerosis 10:493-6.
19. Boden G 1999 Free fatty acids, insulin resistance, and type 2 diabetes mellitus. Proc Assoc Am Physicians 111:241-8.
20. Shimomura L Funahashi T, Takahashi M, Maeda K, Kotani K, Nakamura T, Yamashita S, Miura M, Fukuda Y, Takemura K, Tokunaga K, Matsuzawa Y 1996 Enhanced expression of PAI-1 in visceral fat: possible contributor to vascular disease in obesity. Nat Med 2:800-3.
21. Alessi M C, Peiretti F, Morange P, Henry M, Nalbone G, Juhan-Vague I 1997 Production of plasminogen activator inhibitor I by human adipose tissue: possible link between visceral fat accumulation and vascular disease. Diabetes 46:860-7.
22. Petersen K F, Befroy D, Dufour S, Dziura J, Ariyan C, Rothrnan D L, DiPietro L, Cline G W, Shulman G I 2003 Mitochondrial dysfunction in the elderly: possible role in insulin resistance. Science 300:1140-2
23. Tatar M, Bartke A, Antebi A 2003 The endocrine regulation of aging by insulin-like signals. Science 299:1346-51
24. Brownlee M. 2001 Biochemistry and molecular cell biology of diabetic complications. Nature 414:813-20
25. Cooksey R C, McClain D A 2002 Transgenic mice overexpressing the rate-limiting enzyme for hexosamine synthesis in skeletal muscle or adipose tissue exhibit total body insulin resistance. Ann N Y Acad Sci 967:102-11.
26. Evans J L, Goldfine I D, Maddux B A, Grodsky G M 2003 Are oxidative stress-activated signaling pathways mediators of insulin resistance and beta-cell dysfunction? Diabetes 52:1-8
27. Koya D, King G L 1998 Protein kinase C activation and the development of diabetic complications. Diabetes 47:859-66.
28. King R H 2001 The role of glycation in the pathogenesis of diabetic polyneuropathy. Mol Pathol 54:400-8
29. Schmidt A M, Yan S D, Yan S F, Stem DM 2001 The multiligand receptor RAGE as a progression factor amplifying immune and inflammatory responses. J Clin Invest 108:949-55.
30. Evans J L, Goldfine I D, Maddux B A, Grodsky GM 2002 Oxidative stress and stress-activated signaling pathways: a unifying hypothesis of type 2 diabetes. Endocr Rev 23:599-622
31. Kang, H. T., J. W. Ju, et al. (2003). “Down-regulation of Sp1 activity through modulation of O-glycosylation by treatment with a low glucose mimetic, 2-deoxyglucose.” J Biol Chem 278(51): 51223.
31a. Nishikawa T, Edelstein D, Du XL, Yamagishi S, Matsumura T, Kaneda Y, Yorek M A, Beebe D, Oates P J, Hanmes H P, Giardino I, Brownlee M 2000 Normalizing mitochondrial superoxide production blocks three pathways of hyperglycaeniic damage. Nature 404:787-90.
32. Nadler S T, Stoehr J P, Schueler K L, Tanimoto G, Yandell B S, Attie A D 2000 The expression of adipogenic genes is decreased in obesity and diabetes mellitus. Proc Natl Acad Sci USA 97:11371-6.
33. Schwartz M W, Woods S C, Porte D, Jr., Seeley R J, Baskin D G 2000 Central nervous system control of food intake. Nature 404:661-71.
34. Barsh G S, Farooqi I S, O'Rahilly S 2000 Genetics of body-weight regulation. Nature 404:644-51.
35. Lowell B B, Spiegelman B M 2000 Towards a molecular understanding of adaptive thermogenesis. Nature 404:652-60
36. Saper C B, Chou T C, Elmquist J K 2002 The need to feed: homeostatic and hedonic control of eating. Neuron 36:199-211
37. Friedman J M, Halaas J L 1998 Leptin and the regulation of body weight in mammals. Nature 395:763-70
38. Bates S H, Stearns W H, Dundon T A, Schubert M, Tso A W, Wang Y, Banks A S, Layery H J, Haq A K, Maratos-Flier E, Neel B G, Schwartz M W, Myers M G, Jr. 2003 STAT3 signalling is required for leptin regulation of energy balance but not reproduction. Nature 421:856-9.
39. Shimada M, Tritos N A, Lowell BB, Flier JS, Maratos-Flier E 1998 Mice lacking melanin-concentrating hormone are hypophagic and lean. Nature 396:6704.
40. Huszar D, Lynch C A, Fairchild-Huntress V, Dumore J H, Fang Q, Berkemeier L R, Gu W, Kesterson R A, Boston BA, Cone R D, Smith F J, Campfield L A, Burn P, Lee F 1997 Targeted disruption of the melanocortin-4 receptor results in obesity in mice. Cell 88:131-41.
41. Branson R, Potoczna N, Kral J G, Lentes K U, Hoehe M R, Horber F F 2003 Binge eating as a major phenotype of melanocortin 4 receptor gene mutations. N Engl J Med 348:1096-103.
42. Ahima R S, Prabakaran D, Mantzoros C, Qu D, Lowell B, Maratos-Flier E, Flier J S 1996 Role of leptin in the neuroendocrine response to fasting. Nature 382:250-2.
43. Zhang Y, Proenca R, Maffei M, Barone M, Leopold L, Friedman J M 1994 Positional cloning of the mouse obese gene and its human homologue. Nature 372:425-32.
44. Chen H, Charlat O, Tartaglia L A, Woolf E A, Weng X, Ellis S J, Lakey N D, Culpepper J, Moore K J, Breitbart R E, Duyk G M, Tepper R1, Morgenstern J P 1996 Evidence that the diabetes gene encodes the leptin receptor: identification of a mutation in the leptin receptor gene in db/db mice. Cell 84:491-5.
45. Montague C T, Farooqi I S, Whitehead J P, Soos M A, Rau H, Wareham N J, Sewter C P, Digby J E, Mohammed S N, Hurst JA, Cheetham C H, Earley A R, Barnett A H, Prins J B, O'Rahilly S 1997 Congenital leptin deficiency is associated with severe early-onset obesity in humans. Nature 387:903-8.
46. Ozata M, Ozdemir I C, Licinio J 1999 Human leptin deficiency caused by a missense mutation: multiple endocrine defects, decreased sympathetic tone, and immune system dysfunction indicate new targets for leptin action, greater central than peripheral resistance to the effects of leptin, and spontaneous correction of leptin-mediated defects. J Clin Endocrinol Metab 84:3686-95.
47. Halaas J L, Boozer C, Blair-West J, Fidahusein N, Denton D A, Friedman J M 1997 Physiological response to long-term peripheral and central leptin infusion in lean and obese mice. Proc Natl Acad Sci USA 94:8878-83
48. Saltiel A R, Kahn C R 2001 Insulin signalling and the regulation of glucose and lipid metabolism. Nature 414:799-806.
49. Kitamura T, Kahn C R, Accili D 2003 Insulin receptor knockout mice. Annu Rev Physiol 65:313-32
50. Abel ED, Peroni O, Kim J K, Kim Y B, Boss O, Hadro E, Minnemann T, Shulman G L Kahn B B 2001 Adipose-selective targeting of the GLUT4 gene impairs insulin action in muscle and liver. Nature 409:729-33.
51. Lefebvre A M, Laville M, Vega N, Riou J P, van Gaal L, Auwerx J, Vidal H 1998 Depot-specific differences in adipose tissue gene expression in lean and obese subjects. Diabetes 47:98-103.
52. Flier J S 1995 The adipocyte: storage depot or node on the energy information superhighway? Cell 80:15-8.
53. Steppan C M, Bailey S T, Bhat S, Brown E J, Banerjee R R, Wright C M, Patel H R, Ahima R S, Lazar M A 2001 The hormone resistin links obesity to diabetes. Nature 409:307-12
54. Scherer P E, Williams S, Fogliano M, Baldini G, Lodish H F 1995 A novel serum protein similar to C1q, produced exclusively in adipocytes. J Biol Chem 270:26746-9.
55. Kondo H, Shimomura L Matsukawa Y, Kumada M, Takahashi M, Matsuda M, Ouchi N, Kihara S, Kawamoto T, Sumitsuji S, Funahashi T, Matsuzawa Y 2002 Association of adiponectin mutation with type 2 diabetes: a candidate gene for the insulin resistance syndrome. Diabetes 51:2325-8.
56. Hotta K, Funahashi T, Bodkin N L, Ortmeyer H K, Arita Y, Hansen BC, Matsuzawa Y 2001 Circulating concentrations of the adipocyte protein adiponectin are decreased in parallel with reduced insulin sensitivity during the progression to type 2 diabetes in rhesus monkeys. Diabetes 50:1126-33.
57. Combs T P, Berg A H, Obici S, Scherer P E, Rossetti L 2001 Endogenous glucose production is inhibited by the adipose-derived protein Acrp30. J Clin Invest 108:1875-81.
58. Berg A H, Combs T P, Du X, Brownlee M, Scherer P E 2001 The adipocyte-secreted protein Acrp30 enhances hepatic insulin action. Nat Med 7:947-53.
59. Kubota N, Terauchi Y, Yamauchi T, Kubota T, Moroi M, Matsui J, Eto K, Yamashita T, Kamon J,. Satoh H, Yano W, Froguel P., Nagai R, Kimura S, Kadowaki T, Noda T 2002 Disruption of adiponectin causes insulin resistance and neointimal formation. J Biol Chem 277:25863-6.
60. Ma K, Cabrero A, Saha P K, Kojima H, Li L, Chang B H, Paul A, Chan L 2002 Increased beta-oxidation but no insulin resistance or glucose intolerance in mice lacking adiponectin. J Biol Chem 277:34658-61.
61. Baldo A, Sniderman A D, St-Luce S, Avramoglu R K, Maslowska M, Hoang B, Monge J C, Bell A, Mulay S, Cianflone K 1993 The adipsin-acylation stimulating protein system and regulation of intracellular triglyceride synthesis. J Clin Invest 92:1543-7.
62. Flier J S, Cook K S, Usher P, Spiegelman B M 1987 Severely impaired adipsin expression in genetic and acquired obesity. Science 237:405-8.
63. Hotamisligil G S, Murray D L, Choy L N, Spiegelman B M 1994 Tumor necrosis factor alpha inhibits signaling from the insulin receptor. Proc Natl Acad Sci USA 91:4854-8.
64. Hotamisligil G S, Shargill N S, Spiegelman B M 1993 Adipose expression of tumor necrosis factor-alpha: direct role in obesity-linked insulin resistance. Science 259:87-91.
65. Uysal K T, Wiesbrock S M, Marino M W, Hotamisligil G S 1997 Protection from obesity-induced insulin resistance in mice lacking TNF-alpha function. Nature 389:610-4.
66. Pick A, Clark J, Kubstrup C, Levisetti M, Pugh W, Bonner-Weir S, Polonsky K S 1998 Role of apoptosis in failure of beta-cell mass compensation for insulin resistance and beta-cell defects in the male Zucker diabetic fatty rat. Diabetes 47:358-64.
67. Tokuyama Y, Sturis J, DePaoli A M, Takeda J, Stoffel M, Tang J, Sun X, Polonsky K S, Bell G I 1995 Evolution of beta-cell dysfunction in the male Zucker diabetic fatty rat. Diabetes 44:1447-57.
68. Withers D J, Gutierrez J S, Towery H, Burks D J, Ren J M, Previs S, Zhang Y, Bemal D, Pons S, Shulman G L Bonner-Weir S, White M F 1998 Disruption of IRS-2 causes type 2 diabetes in mice. Nature 391:9004
69. Laybutt D R, Weir G C, Kaneto H, Lebet J, Palmiter R D, Sharma A, Bonner-Weir S 2002 Overexpression of c-Myc in beta-cells of transgenic mice causes proliferation and apoptosis, downregulation of insulin gene expression, and diabetes. Diabetes 51:1793-804.
70. Laybutt D R, Sharna A, Sgroi D C, Gaudet J, Bonner-Weir S, Weir GC 2002 Genetic regulation of metabolic pathways in beta-cells disrupted by hyperglycemia. J Biol Chem 277:10912 21.
71. Laybutt D R, Glandt M, Xu G, Ahn Y B, Trivedi N, Bonner-Weir S, Weir G C 2003 Critical reduction in beta-cell mass results in two distinct outcomes over time. Adaptation with impaired glucose tolerance or decompensated diabetes. J Biol Chem 278:2997-3005.
72. Rosen E D, Spiegelman B M 2001 PPARg: a nuclear regulator of metabolism, differentiation, and cell growth. J Biol Chem 276:37731-4.
73. Zhang C Y, Baffy G, Perret P, Krauss S, Peroni 0, Grujic D, Hagen T, Vidal-Puig A J, Boss O, Kim Y B, Zheng X X, Wheeler M B, Shulman G I, Chan C B, Lowell B B 2001 Uncoupling protein-2 negatively regulates insulin secretion and is a major link between obesity, beta cell dysfunction, and type 2 diabetes. Cell 105:745-55.
74. Clapham J C, Arch J R, Chapman H, Haynes A, Lister C, Moore G B, Piercy V, Carter S A, Lehner I, Smith S A, Beeley L J, Godden R J, Herrity N, Skehel M, Changani K K, Hockings P D, Reid D G, Squires S M, Hatcher J, Trail B, Latcham J, Rastan S, Harper A J, Cadenas S, Buclingham J A, Brand M D, Abuin A 2000 Mice overexpressing human uncoupling protein-3 in skeletal muscle are hyperphagic and lean. Nature 406:415-8.
75. Surwit R S, Feinglos M N, Rodin J, et al. 1995 Differential effects of fat and sucrose on the development of obesity and diabetes in C57BL/6J and A/J mice. Metabolism 44:645-51.
76. Lee Y, Wang M Y, Kakuma T, et al. 2001 Liporegulation in diet-induced obesity. The antisteatotic role of hyperleptinemia. J Biol Chem 276:5629-35.
77. Tang H, Vasselli J R, Wu E X, Boozer C N, Gallagher D 2002 High-resolution magnetic resonance imaging tracks changes in organ and tissue mass in obese and aging rats. Am J Physiol Regul Integr Comp Physiol 282:R890-9.
78. Makimura H, Mizuno T M, Beasley J, Silverstein J H, Mobbs C V 2003 Adrenalectomy stimulates hypothalamic proopiomelanocortin expression but does not correct diet-induced obesity. BMC Physiol 3:4
79. Thupari J N, Landree L E, Ronnett G V, Kuhajda F P 2002 C75 increases peripheral energy utilization and fatty acid oxidation in diet-induced obesity. Proc Natl Acad Sci USA 99:9498-502.
80. Kumar MV, Shimokawa T, Nagy T R, Lane M D 2002 Differential effects of a centrally acting fatty acid synthase inhibitor in lean and obese mice. Proc Natl Acad Sci USA 99:1921-5
81. Fu J, Gaetani S, Oveisi F, et al. 2003 Oleylethanolamide regulates feeding and body weight through activation of the nuclear receptor PPAR-alpha. Nature 425:90-3.
82. Razani B, Combs T P, Wang X B, et al. 2002 Caveolin-1-deficient mice are lean, resistant to diet-induced obesity, and show hypertriglyceridemia with adipocyte abnormalities. J Biol Chem 277:863547.
83. Li J, Takaishi K, Cook W, McCorkle S K, Unger R H 2003 Insig-1 “brakes” lipogenesis in adipocytes and inhibits differentiation of preadipocytes. Proc Natl Acad Sci USA 100:9476-81
84. Bush EN, Shapiro R Nuss M E, et al. 2001 Adiposity, Leptin Resistance, Hyperrphagia, Hyperglycemia, Glucose Intolerance and Insulin Resistance in C57BL/6J Mice Fed High Fat Diets. Endocrine SocietyAnnual Meeting 2001, Poster Session.
85. Lupien, S. J., M. de Leon, et al. (1998). “Cortisol levels during human aging predict hippocampal atrophy and memory deficits.” Nat Neurosci 1(1): 69-73.
86. Rajala, M. W. and P. E. Scherer (2003). “Minireview: The adipocyte—at the crossroads of energy homeostasis, inflammation, and atherosclerosis.” Endocrinology 144(9): 3765-73.
87. Chaldakov, G. N., I. S. Stankulov, et al. (2003). “Adipobiologyofdisease: adipokines and adipokine-targeted pharmacology.” Curr Pharm Des 9(12): 1023-31.
88. Anderson L, Seilhamer J 1997 A comparison of selected mRNA and protein abundances in human liver. Electrophoresis 18:533-7.
89. McCall, A. L. (2002). “Diabetes mellitus and the central nervous system.” Int Rev Neurobiol 51: 415-53.
90. Starr, J. M., J. Wardlaw, et al. (2003). “Increased blood-brain barrier permeability in type II diabetes demonstrated by gadolinium magnetic resonance imaging.” J Neurol Neurosurg Psychiatry 74(1): 76-6.
91. Kohn, D. T., K. C. Tsai, et al. (1996). “Role of highly conserved pyrimidine-rich sequences in the 3′ untranslated region of the GAP43 mRNA in mRNA stability and RNA-protein interactions.” Brain Res Mol Brain Res 36(2): 240-50.
92. Perrone-Bizzozero, N. I., V. V. Cansino, et al. (1993). “Posttranscriptional regulation of GAP-43 gene expression in PC12 cells through protein kinase C-dependent stabilization of the mRNA.” J Cell Biol 120(5): 1263-70.
93. Okada, S., W. Y. Chen, et al. (1992). “A growth hormone (GH) analog can antagonize the ability ofnative GH to promote differentiation of 3T3-F442A preadipocytes and stimulate insulin-like and lipolytic activities in primary rat adipocytes.” Endocrinology 130(4): 2284-90.
94. Brewer G, Ross J 1989 Regulation of c-myc mRNA stability in vitro by a labile destabilizer with an essential nucleic acid component. Mol Cell Biol 9:1996-2006.
95. Rao, A. and O, Steward (1991). “Evidence that protein constituents of postsynaptic membrane specializations are locally synthesized: analysis of proteins synthesized within synaptosomes.” J Neurosci 11(9): 2881-95.
96. Weiler, I. J. and W. T. Greenough (1993). “Metabotropic glutamate receptors trigger postsynaptic protein synthesis.” Proc Natl Acad Sci USA 90(15): 7168-71.
97. Expert-Bezancon, A., J. P. Le Caer, et al. (2002). “Heterogeneous nuclear ribonucleoprotein (hnRNP) K is a component of an intronic splicing enhancer complex that activates the splicing of the alternative exon 6A from chicken beta-tropomyosin pre-mRNA.” J Biol Chem 277(19): 16614-23
98. Kopchick J J, List E O, Kohn D T, Keidan G M, Qiu L, Okada S 2002 Perspective: proteomrics-see “spots” run. Endocrinology 143:19904
99. Pandey, A. and M. Mann (2000). “Proteomics to study genes and genomes.” Nature 405(6788): 837-46.
100. Naaby-Hansen, S., M. D. Waterfield, et al. (2001). “Proteomics-post-genomic cartography to understand gene function.” Trends Pharmacol Sci 22(7): 376-84.
101. Klose, J., C. Nock, et al. (2002). “Genetic analysis of the mouse brain proteome.” Nat Genet. 30(4): 385-93.
102. Lopez M F, Berggren K, Chemokalskaya E, Lazarev A, Robinson M, Patton W F 2000 A comparison of silver stain and SYPRO Ruby Protein Gel Stain with respect to protein detection in two-dimensional gels and identification by peptide mass profiling. Electrophoresis 21:3673-83.
103. Lauber W M, Carroll JA, Dufield D R, Kiesel J R, Radabaugh M R, Malone J P 2001 Mass spectrometry compatibility of two-dimensional gel protein stains. Electrophoresis 22:906-18.
104. Malone JP, Radabaugh M R, Leimgruber R M, Gerstenecker G S 2001 Practical aspects of fluorescent staining for proteomic applications. Electrophoresis 22:919-32.
105. Celis J E, Gromrov P, Ostergaard M, Madsen P, Honore B, Dejgaard K, Olsen E, Vornm H, Kristensen D B, Gromova I, Haunso A, Van Damme J, Puype M, Vandekerckhove J, Rasmussen H H 1996 Human 2-D PAGE databases for proteome analysis in health and disease: http://biobase.dk/cgi-bin/celis. FEBS Lett 398:129-34.
106. Sanchez J C, Chiappe D, Converset V, Hoogland C, Binz P A, Paesano S, Appel R D, Wang S, Sennitt M, Nolan A, Cawthome M A, Hochstrasser D F 2001 The mouse SWISS-2D PAGE database: a tool for proteomics study of diabetes and obesity. Proteomics 1:136-63.
107. Aebersold, R. and M. Mann (2003). “Mass spectrometry-based proteomics.” Nature 422(6928): 198-207.
108. Mann, M. and O. N. Jensen (2003). “Proteomic analysis of post-translational modifications.” Nat Biotechnol 21(3): 255-61.
109. Steen, H. and A. Pandey (2002). “Proteomics goes quantitative: measuring protein abundance.” Trends Biotechnol 20(9): 361A4.
110. Kriete A, Anderson M K, Love B, et al. 2003 Combined histomorphometric and gene-expression profiling applied to toxicology. Genome Biol 4.
111. Taylor, C. F., N. W. Paton, et al. (2003). “A systematic approach to modeling, capturing, and disseminating proteomics experimental data.” Nat Biotechnol 21(3): 247-54.
112. Orchard, S., H. Hermjakob, et al. (2003). “The proteomics standards initiative.”

Proteomics 3(7): 1374-6.
113. Garrels, J. I., C. S. McLaughlin, et al. (1997). “Proteome studies of Saccharomyces cerevisiae: identification and characterization of abundant proteins.” Electrophoresis 18(8): 1347-60.
114. Futcher, B., G. I. Latter, et al. (1999). “A sampling of the yeast proteome.” Mol Cell Biol 19(11): 7357-68.
115. Gavin, A. C., M. Bosche, et al. (2002). “Functional organization of the yeast proteome by systematic analysis of protein complexes.” Nature. 415(6868): 141-7
116. Patterson, S. D. (2003). “Data analysis—the Achilles heel of proteomics.” Nat Biotechnol 21(3): 221-2.
117. Reis, B. Y., A. S. Butte, et al. (2001). “Extracting knowledge from dynamics in gene expression.” J Biomed Inform 34(1): 15-27.
118. Butte, A. J., P. Tamayo, et al. (2000). “Discovering functional relationships between RNA expression and chemotherapeutic susceptibility using relevance networks.” Proc Natl Acad Sci USA 97(22): 12182-6.
119. Ramoni, M. F., P. Sebastiani, et al. (2002). “Cluster analysis of gene expression dynamics.” Proc Natl Acad Sci USA 99(14): 9121-6.
120. Zhang, N., R. Aebersold, et al. (2002). “ProbID: a probabilistic algorithm to identify peptides through sequence database searching using tandem mass spectral data.” Proteomics 2(10): 1406-12.

Citation of documents herein is not intended as an admission that any of the documents cited herein is pertinent prior art, or an admission that the cited documents is considered material to the patentability of any of the claims of the present application. All statements as to the date or representation as to the contents of these documents is based on the information available to the applicant and does not constitute any admission as to the correctness of the dates or contents of these documents.

The appended claims are to be treated as a non-limiting recitation of preferred embodiments.

All references cited herein, including journal articles or abstracts, published, corresponding, prior or otherwise related U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

In addition to those set forth elsewhere, the following references are hereby incorporated by reference, in their most recent editions as of the time of filing of this application: Kay, Phage Display of Peptides and Proteins: A Laboratory Manual; the John Wiley and Sons Current Protocols series, including Ausubel, Current Protocols in Molecular Biology; Coligan, Current Protocols in Protein Science; Coligan, Current Protocols in Immunology; Current Protocols in Human Genetics; Current Protocols in Cytometry; Current Protocols in Pharmacology; Current Protocols in Neuroscience; Current Protocols in Cell Biology; Current Protocols in Toxicology; Current Protocols in Field Analytical Chemistry; Current Protocols in Nucleic Acid Chemistry; and Current Protocols in Human Genetics; and the following Cold Spring Harbor Laboratory publications: Sambrook, Molecular Cloning: A Laboratory Manual; Harlow, Antibodies: A Laboratory Manipulating the Mouse Embryo: A Laboratory Manual; Methods in Yeast Genetics: A Cold Spring Harbor Laboratory Course Manual; Drosophila Protocols; Imaging Neurons: A Laboratory Manual; Early Development of Xenopus laevis: A Laboratory Manual; Using Antibodies: A Laboratory Manual; At the Bench: A Laboratory Navigator; Cells: A Laboratory Manual; Methods in Yeast Genetics: A Laboratory Course Manual; Discovering Neurons: The Experimental Basis of Neuroscience; Genome Analysis: A Laboratory Manual Series; Laboratory DNA Science; Strategies for Protein Purification and Characterization: A Laboratory Course Manual; Genetic Analysis of Pathogenic Bacteria: A Laboratory Manual; PCR Primer: A Laboratory Manual; Methods in Plant Molecular Biology: A Laboratory Course Manual; Manipulating the Mouse Embryo: A Laboratory Manual; Molecular Probes of the Nervous System; Experiments with Fission Yeast: A Laboratory Course Manual; A Short Course in Bacterial Genetics: A Laboratory Manual and Handbook for Escherichia coli and Related Bacteria; DNA Science: A First Course in Recombinant DNA Technology; Methods in Yeast Genetics: A Laboratory Course Manual; Molecular Biology of Plants: A Laboratory Course Manual

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Any description of a class or range as being useful or preferred in the practice of the invention shall be deemed a description of any subclass (e.g., a disclosed class with one or more disclosed members omitted) or subrange contained therein, as well as a separate description of each individual member or value in said class or range.

The description of preferred embodiments individually shall be deemed a description of any possible combination of such preferred embodiments, except for combinations which are impossible (e.g., mutually exclusive choices for an element of the invention) or which are expressly excluded by this specification.

If an embodiment of this invention is disclosed in the prior art, the description of the invention shall be deemed to include the invention as herein disclosed with such embodiment excised.

1. A method of protecting a human subject from progression from a normoinsulinemic state to a hyperinsulinemic state, or from either to a type II diabetic state, which comprises administering to the subject a protective amount of an agent which is
(I)
(1) a polypeptide which is substantially structurally identical or conservatively identical in sequence to a reference protein which is selected from the group consisting of mouse and human proteins set forth in master table 1, subtables 1A and 1C,
or
(2) an expression vector comprising a coding sequence encoding the polypeptide of (1) above and expressible in a human cell, under conditions conducive to expression of the polypeptide of (1);
or
(II)
(1) an antagonist of a polypeptide, occurring in said subject, which is substantially structurally identical or conservatively identical in sequence to a reference protein which is selected from the group consisting of mouse and human proteins set forth in master table 1, subtable 1B and 1C, table 2, subtables 2B and 2C,
(2) a nucleic acid molecule which inhibits expression of said polypeptide in said subject,
where said agent protects said subject from progression from a normoinsulinemic state to a hyperinsulinemic state, or from either to a type II diabetic state.

2. A method of screening for human subjects who are prone to progression from a normoinsulinemic state to a hyperinsulinemic state, or from either to a type II diabetic state, which comprises assaying tissue or body fluid samples from said subjects to determine the level of expression of a human marker gene,
said marker gene being either
(I) a “favorable” human marker gene, said human marker gene encoding a human protein which is substantially structurally identical or conservatively identical in sequence to a reference protein which is selected from the group consisting of mouse and human proteins set forth in master table 1, subtables 1A and 1C,
or
(II) an “unfavorable” human marker gene, said human marker gene encoding a human protein which is substantially structurally identical or conservatively identical in sequence to a reference protein which is selected from the group consisting of mouse and human proteins set forth in master table 1, subtable 1B and 1C,
and correlating the level of expression of said marker gene with the propensity to progression in said patient, said correlation being direct if the marker gene is “favorable” and inverse if the marker gene is “unfavorable”.

3. The method of claim 1 in which (I) applies.

4. The method of claim 3 in which the reference protein is of subtable 1A.

5. The method of claim 1 in which (II) applies.

6. The method of claim 5 in which the reference protein is of subtable 1B.

7. The method of claim 2 in which (I) applies.

8. The method of claim 7 in which the reference protein is of subtable 1A.

9. The method of claim 2 in which (II) applies.

10. The method of claim 9 in which the reference protein is of subtable 1B.

11. The method of claim 1 in which the reference protein is a human protein, and that human protein is the top scoring human protein in Master Table 1 with respect to sequence homology with one of the differentially expressed mouse proteins listed in Master Table 1.

12. The method of claim 1 in which the reference protein is a human protein.

13. The method of claim 1 in which the reference protein is a mouse protein.

14. The method of claim 2 in which the level of expression of the marker protein is ascertained by measuring the level of the corresponding messenger RNA.

15. The method of claim 2 in which the level of expression is ascertained by measuring the level of a protein encoded by said marker gene.

16. The method of claim 1 in which said polypeptide is at least 80% identical or is at least highly conservatively identical to said reference protein.

17. The method of claim 1 in which said polypeptide is at least 90% identical to said reference protein.

18. The method of claim 1 in which said polypeptide is at least 95% identical to said reference protein.

19. The method of claim 1 in which said polypeptide is identical to said reference protein.

20. The method of claim 1 in which the best E-value cited for the reference protein in Master Table 1 is not more than e-50.

21. The method of claim 20 in which the best E-value cited for the reference protein in Master Table 1 is less than e-80.

22. The method of claim 1 in which the antagonist is an antibody, or an antigen-specific binding fragment of an antibody.

23. The method of claim 1 in which the antagonist is a peptide, peptoid, nucleic acid, or peptide nucleic acid oligomer.

24. The method of claim 1 in which the antagonist is an organic molecule with a molecular weight of less than 500 daltons.

25. The method of claim 24 which said organic molecule is identifiable as a molecule which binds said polypeptide by screening a combinatorial library.

26. The method of claim 25 in which said organic molecule is a heterocyclic organic compound which is
(1) a cyclic compound containing one heteroatom which is heteronitrogen, or
(2) a cyclic compound containing one heteroatom which is heterooxygen, or
(3) a cyclic compound containing one heteroatom which is heterosulfur; or
(4) a cyclic compound, with two or more hetero atoms.

27. The method of claim 1 in which said reference protein is a human protein and the polypeptide is identical to said human protein.

28. The method of any claim 1 in which said reference protein is a mouse protein and the polypeptide is identical to said mouse protein.
